SEQUENCE LISTING (14 sequences)

SEQ ID NO: 1 - DNA, 28394 bp, Homo Sapien
acacagagcagagtggggctctgagtatataactgttaggtgcctccctccagcaccatctcctgagaagcactctcccttgtcgtggaggtgggcaaatctttatcagccactgccttctgctgccaggaagccagctagagtggtgtaagtactcatccttatttctattcatttccaactattcatcatttggggcttgtcttcacagttctaagttttgctctttttcttaatgaagaaaatgttttatatcaccggaattgatcagaagtagcaaaatcagagttctggtagactagaaagcaatttaccaaagccacaggcttcttcctggaagctcaaaggcatgcctttattcgtgatttctgaagcaaggtgcatgcagcacctgagctgatgtggaagagggtttgcagggaggtgtccacccaatgtgctcaatgattctgggttaatcaacactattaggagtttcaggttgtgttcttgaaataataatttgggctgtgttcttgaaataagttcgaggcgagtgtctacaagactcaaaagaaaaaagtgggccactgggaatggccctttccagtgatggatttatggactcctctgtgtgtgctgtcatgctgaagggaatgttcttgtgcacccatcgggagaacaagtcagtcacaactgaagccacgaatttggcagcttccttgcagctgcactctctggagtctggaatcaagacttctgggagtagtgttttccaaggagggaagtgttttaaccaggacacaggaatatctgacagcattttctttgtttccaattacagctttaaagaaaactgggcatctcctgctacttaaaatcaaaaactacctaaaataaagattatagtaagtaccaaataagtgtcaatgctgaaagtctctttattatgctagaccatgagtgtttaaatgctttcttctatatccatatccaacacttcatattatttttaaaagtaatagctgaagcatggaaaattgaagacttcaggtctctccaattgcacaaatttctaatacatgctggcaatagaatatattttatttcgtgtaataaaatagaggatattagttgacctgaaatcttgatattgccttgtattaaaatgctaagcactgcttcattttactagtgatctggggtatgaaaagtgctttttgacttctgctggaaagctcttcaggtgcagcttccaggatattcttgggatgttaacttcagcacacataagccttgctgtagatgtgtcagctttgaggcacagggagacatttgtttgtcagagagtaactgcttctggcaagggcatagggtgaaactggggatagcagagctctttctttgtggttgttcaacccccaccccaagattagttcaaagtgaccgtgaagatagtctgtgcccaccgcatcgctaagtcctagccctctctgcatactccagcacacagaaactgctgcttcacttgtttgttgacttgaaccgaaccttgggtggcattaatgtgcctggcccaagactgaaaaattaagaaccaccagagctgacctattccataagacccagtctgcctgccacgtactgagtgaatctggatgatgcccactctgatccttggttttctcttctataaaatgaaggcttgaactacgtggtctctaaaatcctacctagctctcaaatttctcttggttctaggaaaatattgatgttgagctcaaggaaggggttctccaaggtgtgtgattttggtggtagaggaaaggccggtgccaggcaggggcagaaggagacgctgtctacactgagaaaatgtgacaacccctgcttgtctcttttttcattcttcattgtttcttatttctttgtttttagctttatataacatgagagccctaccactgggtttcttaaccatttgttctttatcaaataaaaatattcataatgcaacatgcaggcacatcagtgtggtacagaactagccagctagtttactataggtaaatatacacacatgcatgcacacacacaatttttacctgagacatgtcagaagtgtttcctaaaattgtggatttttctgagtcattctggtaaagggtaggttttcaggttttaggccaagccagaagaagaaagtaaaaacagaataaacaacagggggagaaaaagagaaataccacacacacaactggaacttctggtaaaagagtgatattcttggatgcaatggaagttttaaaaaggaaaaagaaaatttataaaaagctgccacatttgtggaattcaactaaaaactgtttattattaacaaagtgatgttcaaaatttaagagttcttggcctggcatgatgacttatgcctgtaatcccagtgttttgggaggctaaggtgggaggatcacttgaggccaggaattcaaaaccagcctggacaatacaatgagactttgtctctaaaaaaaaataaaataaattaaaataaacacagctggatgtggtggcacaggaaaaaaaaataccatttaggagtctcttaaaggcagcttgtgaatgcttacaaagcgtggctagtatcttattacagaaaacagagcccacatcatgcatccttcttctcacatttcataaacaaggccaagggaaactgctgtggggcaacctgttgctttggtgttggtccccaagatgcagccctcacaatctgcccccaaacgtgtcagaacatgaaccccctcctccccctctggaagaagcaacctcagatccaacagcagagacacgcagcagaacaaaatctgggcattggtccctgtgtaggatggcttcccgttattttttttttaagcaaagtaaatgaacatcaaatttccatagtcagctgctgtctttctgcccactgagagctctttggtgaaggcaaagtcctccttcttcattagcggtctcccatgtggggccacatcttccctcaccaggaacccagtgggcgcgctccagcccccctcagcttgccttttgcgtggtcattagagctagggcacacgtcatgctgattcacatatttttgccctttgtcatgtattgagaaaaagtaaggatgaatggacggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggtatgcttccttcgtgcagcctctggctcggggaacctgggagcctgctccaaactctggtgtatcttttccgggcagagcctgggaagtgggggttggctgtgagctaagccaaaggcacagggatcttggtccaaaaagccccatggcgctcaccttggtttagaggctagaccattgagctgagaagttttgacagccatggaaaagctggggataagtcacctggggttttacgtttaccctgtgtctattttattagagtgccttttacttattgtcccttcttcttagttgaaattaatggcctgcttcactggggctaagatgtttgaacattagcagaaggtcctggctgcatagccttgccttgtcttcccagttaggatgtaaggactcttaaagttccctaagaaatgcaaatattttagcatggcaaaattctaggccaactacaactgtaagtttcgtatttctcctaagtggttctcatgcctgacttctggagcaaggagtcaggtctcccaggggctctagaagggttcagctgttcagaataaatggttcctggggactctaaaatagcagcaactgtctgcccaggtcatgagaagacccctctctgcaggacatcctagccctacaacccatcccaattatgttgaaattagattcacaaatggcaataagtcttctatatgttgggctgtcgatttggagaaaactagtttaatctttacttaactttgggtggctcaacaggagactcgggccgctcaggctctcaatcacgtctggccagttctattatcaggtttcgaatctgtatctccaaaatctctgaggtgatgggatatttcaagccctctaaaataaataaatatatgctgggaattttgagaacatgaatttgtttattctgaaatggtccatgttcctgctttgggagttgatggaaaatgccacttgagtgttttcatttgatgctgccaccttagggttttatagattcagttccagaaactcaaggcatttatctctttgggctgcttgtccttgcctgagctgaagcctgatgcctcccataagttggtatggctttgaaaatgggtcactacagcagaggcatgggcttatcaagcaatatgttcagctatgaaatttgaagagggagataatctgaaaataaatgacagccaccacttagattatgaaatagaagtactttttcataagtgcttaattattcatacggttttttatctttaactatggagccaactcagctccatatggacttaattttggttcctgacctccaagattcattgcaagtcacacagatgttggtatctaacattgttttaccgagataaaatgaccttggtctggaatgcattgtataaaaagctgcttttttgtgtaaagattaatagtttggcattgtttaaaaagcagaatggttagttgggcagtgaggtaatacaattgaaatgtaattgctaccaataaatcagttacccatattgatttctttactgggattaatagaagccaaagctagagttcaactttttttaataggtataacttagtatctgttcattgctatttgttagctatggtaaatggaacaatgatggggccagaaatatccatgaggaccatttgatcacagcctggcaacacagagaagacaggctggtttctctatgtgggctttcagtgtttctttggtagtgtcttatgtggctgtggcttcaacattccacaattatgccttccagggtctgatgattttggcgtttccctgcttcccaattgacctggctgtgctgttggctgttcttgcacactcaaggtggttttgccattggcttcctccctcagcctgcctctgggattatgccactgctattcttttttatctaccatcagcacaatgaaatcatcatttttgtcttcaaggtaccaaattctggtgatattggtgctttcttgcagctacttatcatgagaagtgaatggtctcatagtgaacacagtcatggttatagtgttcatacgttccagagacatgtttcctataattatgccctgcacatttttctatcatacaatccttagattacagctctttggttttcaacagctttgtccaattccatctttcccagtttctctaccttgatgaaatatccttcttgcctggttttacatatttaaataacaaattccaaaagtaaagagtatctgaggcagtcacatgacataaggacaaattcaagccatcttggacttgcagagggtggggagaccgtgtcaacacacacaattttaaaaatttcttccctttcaatcttttaaaaacaaaactttttataaaataaaaatgtaatttaaaaaggctacctgtcttggcaagtagctgatcagcctgcattggtgagcaggccattccataacctggtttcttgctccttaattgacagcatggagctaacgtacttaatttcagctctttctacgtgatttgactcattctgttaacattaactgtttttcagtcttctcaactagactgaactccttaagtgcaagaaatacacgcttagtaaatgtttgttggaccagacactgcaccttatgaaattaaagaccagaacattctcatggtagcattacagacactgatggcaaaggtactgtgggatttgggtttggctaataagctctgtggtggtgtttcagaaggaaaatggtgctctcttagttctatggaacatagtggtccagatcttctactgtaaccaggcccaaagctggctaatctggagggctctgccttagggatacttataagctctgtccttccctcaaggagccagaggaagagatagccatggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaaggtatttatcctttcacattttggagagacaggagaagtagctttgggggaaatggtttcctggtacttctacttatacctttagttatattctccaactttttatagatctctttactcaccatttttctacttttatcttttaacctgcaaacctctccatttttttttcttatggagacagtagccagggcccagctcatattagaaggcacctggcttcatcctgtagtttcagtacttaaaacttaaatttattcctttggcttcagaatttgtacctataagcatgaaaataagtgcattagatgctttcaggagcttagattctaggaggggcagtgtgggttgagcatacagtagatagaggctttcagggatctgggtgccactaatgcaacaatgggttgagagagaaatattaaagaaatatcaaaaatgtttcacttccaggaggttttgctgattttgctcagggtgggcctgtggttgaagagtatcacttggcagcttccttagctctgctttacctcatcccttccagacaaacccgtggtgctccagttcattgactggattctccggggcatatcccaagtggtgttcgtcaacaaccccgtcagtggaatcctgattctggtaggacttcttgttcagaacccctggtgggctctcactggctggctgggaacagtggtctccactctgatggccctcttgctcagccaggacaggtaggtgtaccctttcaagccttctcagctcccttctgagacacaggggctgaccagttactgtgggcaacagtgataaaaccacatccttcccaggataaacaacatttagtccacagaactgtttatatttgtttttagtcagaggtcagggaatcagttacagtctcttgctcttgatatctgaataaatggctggtctaaatgatgccagattcttgtggcattacgtgctaaccagaactaagctacaagtatttccctggagaggttctgaagggatcttctttaatgattgataaaattatttgtcgtcagcattctatttgggaaaaagtgcatatgaattcagaaaaagttttagtggcttaataacccccgttatatcttgttgctatgatgagtttaggaaactcattcttcatagacagtgcaaaggtcagctcagctcctggagaaaagaataaccatgaattccaattgagtggattctgacttaagaagccttagtgagtcttctgatatattgattagattaaaaatagcacacactttataaattgatctgtcattgaagaagtgatgagctgactctcaccagggcagtagatagctccccactagccagttcctttagggagggaaccagtattccaggtgtctgagatcaacgcataatcccaatccccagtgtggtcattacacaactaagctcttgtaacactggctgcaaattgcctaaagaggtccgtggggagagagttagcaaatgctccacttttctatcaatttcaaggagtctgatttgctccctgtagaaggggattttatagcttaggttaaactctattccaatgcatgccaagaaaaggtctcctcagtttggggatggagtctataattgtgccatactgaatattcctttatgattttgctctgatgaaacatgatcaactcattttttgtcagatattatttagaagacaagtcatttatatgtgttagtttcaaatgttttactttccttggtctgaaaagactgcattaaaatggaaattctctgttttaagtaaatatatgtcttcctgtggctttaactatggcattccacaatttgtagatgttgccattaattttccactgatcaaactcaagcattaacatctccaagtcagttgttgagaggacaagtctgcatggctctctactgtcatgtgtagtcccagtctctgagttgtacctttgcaaattgtatcacctcccatttgccctcaaggattatttaagggaaacaaagaacttttgaatagggaaccccacatttaatgttcatctggattaatgtacgtgacatcatcttgcctgttgcaatggtgcctcctggcccagttagaaacaagccaagaagcagctgtcacactatcccttaccagcccctgcagtgtggctcactggctatagcacctcctgctcgagcccagcattaggcctcacctactcacttcaccatctttactcccccatccccctacagacatcatccttgagtgacaggcccttgggaagtggatcctgtgcctttcacggtgccagacgttgccaactctcagagctgtgggaatcctgccttgtcaggtcaatcaatctaggtgcccatcaatggtggattatataaagaatatgtggtgcatatacaacacgaactactacatagccataaaaaggattgaaatcaagtcctttgcagcagcatggatgtatctggagaccaatatcctaagtgaattaatgtagtaacagaaaatcaaataccacacgttttcacttacaattaggagctaaacactgggtaaacacggacatggaaatagtagacaactgggactccaaaagaggagaggaagggaaacaagtgttgaaaacctacctatcaggtactttgttcactatttgggtgacgagttcaatagaagcccaaacctcagtcagcatcatgcaatacatctatgtaacaaacctgcacatgtaccccctcaatctaaagaaggagaagaagacggggaagaaatgagattgaatactaagcaaaaagtaacctcagaaagaactgggtgctcaacatgcacataattaaatgggatacttctccaagtaagagaaaagcaattgttcttctttgcaataactttgaaatgtgcgtttggagacaacaaaatagaagcatcaggacacaaaaatgtatactaacctggaagattaatgttgataagatcaaagacactgtgaaagtgaatttacatttcaggaatcttatatctctcaccaagaaatcaaacttaagcaacagtttcatatgctaaaagcgctcttcaagtcagaggctcttgatttaaaagaataactttccaaaggaaaggctaaaagaaaacagagcagattgccttactaaactcccctttcctctcagccactgtagacctgtctttagccgtgacacctgtagagggagtcattctctatcaggggtccccaacccctgcactggagacaggtacctgtctgtggcctgttgggaactgggccgcacagcaggaggtgagcggtgggcgagtgagcatttccacctgagctccgcctcctgtcagatcagcagaagcattagcttctcataagagtgcgaaccccattatgaactgggcatgtgagggatctaggttgcttgctccttatgagaatctaatgcctgataatctgaggtggaacagtttcatcccgaaatcatcccccattccccatccatggaaaattgtcttccatgaaacctgtccctggggccaaaaaggctggggaccactgatctaaatgcacatttatatttttatctatgtatatttcacttcatgtctttattagtttttgtacgatgcttacgtagactttgaaatacatttccaaatataatctcattttttaatatgaatatgatctggaagttactagtgttatttatgtgcaagtgcaaccaaagctcacccaggaaatgtccgtgctgtgtctcttgccccacaggtcattaatagcatctgggctctatggctacaatgccaccctggtgggagtactcatggctgtcttttcggacaagggagactatttctggtggctgttactccctgtatgtgctatgtccatgacttggtaagttacaattggttttcaaaatgcctttttgaaaaaaaaaacatggcagaaggagggaatgggagttgttatatggcagagtttcagttttgcaagatgaaatatgttctctgaatgtatagtggtgatggttgtacaacaatgtgattgtccttaatgtcattgagctgcacacttaaaaatggttagccgggtgcggtggttcttgtttgtagtccaaactattcagaaggctgagggggaaggatcacttgagcccaggagttaggggctgcagtgagctatgattgcgtcaccgcactccagttctccgaacctccttgcttgggctaagtgaggaggaggaggaggaggagaaggatggaaaggaggaggagtagcaggaggagcaggagggcaaggagaaggaggaagaggagcaggaggaggacaaacagttaaaatggtaaatttaaaattggattccagtagattctgtctattggaaacagaaacaaccattttaaaagatgtatatttccttacaaccagttatttggccttttgtctgatctggctacacatccactaatacctctcaaccagaggtggctgcacattgacacttccatggggaagggaaacagtgctgcaatgaagatacgagtgcaggtgtctttttggtagaaacacactgatgcacgtggcccccacatacacttgactcctccctcccaagactctactgtcattggtctgcggtagcgcctgggctttgggagtttctaaagcttcccagatgactctaaagtatagccaaagttgagacccacttcctccatcattgcctctcaaacttgagcaatatgagaatcacctgcagggtttgttacaccacaggcatctgctccccggccccagggtttctgatgcagtctatctggggtggggcccgagaatttgcgtttctaacgcattcccacatgatgctgggagaaccactgtgcctacgtgaattcccccttacccacctgccccccaggtctcccttagaaaaaatttttttgctgaattccttttttttcaaacccaaatccttcaaactagtttttatgttgacaatgtcttacatcctttttctggaaacaaagatttccttctttctatattgtagttaaatataaaatactaatatgcacataaataagcacagcctgctgtgggcagtgtctgcagaagggatgcccacccttactgtacccacgggtgtgtggacgaggacctacctgtagagctaaactcttcaggaagtaatttgggccctgctctgaagaataggttcgtgggaaggaggcctagcctgtaagtgctcaccacgctcccttccacaatccaggaaaatgggagttctggtctttaagtgatggctctttgattgggccaacaagtgagagcctatgagggacctcgggaccatgcagcccagccccacagtttatgggctctgaggctaaggagatgcgccttgcctaggtcatgcaatttatcaacagctcaaggacacacactctgccccaccaactgtgatatcattttcctccagctcacactacctgcatccttgaacgattgtttctcttttccaaaaataggtatattaaagaaataatatctgccaaatcagaatcagggttgcctctagtggggagggagggacataagagcaagtggagggacaaaggggactttaactatgtagataatattttattttgtatgtcataagtacttcaaaaatatttttaaaatctcaatatatagctcactctgagcaaccccagagtagaatttttcaaaagccaaataagctgagagttgattttttactttatgtaatatttactgcctctataataggatttatcccaagttttctttctgtggcaaatgtgccaacacaacacgtaaggggcctgttggcaggtgaaacaaagcccctccagagtatagcgattccgtgtgtcagcctgctttgtcacatgcacattcttttgctctgttctttttttagcccaattttctcaagtgcattgaattccatgctcagcaaatgggacctccccgtcttcaccctccctttcaacatggcgttgtcaatgtacctttcagccacaggacattacaatccattctttccagccaaactggtcatacctataactacagctccaaatatctcctggtctgacctcagtgccctggaggtaagagacactggcttctcacattcgccctggctctgcaagatacgcaatggcctcctggtcaactgtccacgggtgtcagagtctcctagatgctcaggactatggtggcctttctgccttcatcttgccatttaaagcatttgttctactccagagcattagggtctaagggattttttaaaattactatttagtcaagctgatttttctgccttttcccctaaacatctacagtgctaaccccagagtacagttccactgggagtcactctatcgtaagcttgggggtgggggtgatgggagccagcccttaaggcatgtggcctccagcctggttttaaatcttccatagtctactccctccaatcaaaaaactggatgcttactcttagagcttctgacagaacctctctattctgcttttccttatggcatagctcatagaacatctacaataatttagggttcccaagctttggtaggcatcagaatcacctggggagctttaaatacccaaacaggcttcatctcagaccctctaaatcacaatctctaagggtggggcctggaacctgttttaacaaactccccaaattgtgatgcgggccagagtttgagaaccactgtatcaaggggtgaatcctatgtatctctttaaagatggctataaagagattctgtattttttaaaacctggttaacccaaatcaaattccagctcttcctgttggtgtgtaataaatatgtttaaggtttctggattatcaagaacaagagaacacctgaaattagaagaaaaccaaagaaaccttacctttttaatgtgctctcccactgtcaggttatgaaacgcccttttgtcttctttgttgagtgatcaaaacacacgaggagctcaagtcaccttctccctagcttcttgccagaaaactaaagggagcacctggaaataattcagaaggaaaaaatcaaagattcattagaactacccatgaaaaataacagtataaaatagcattaatcgatctagaactgcactaacacaggagcctctagccccatgtggctatataaatttagatgtagattagttaaaaattgagttcctcaacctctctagccacatctcaggtgcttgatagccacacgtggctaggacccactgtattagacagcacagatacagactattccatcatctcggaaagttatcctgcacagtgctgatctggggcaggggaagccttgtccttctcactctgaatgaacagcccatcctcagcaccaaccccaaccctatggctacctgagagagagttctgcagccaagtccaaaaacaaacaaacaaacaaaaaaagcatatgccatctttgccaagttccctggtctagaaatagcaaaatgtctagacatgaagactcagcatgggctggaagaatttagagtccatcttagggtagagtcaaactcacactatggtctggtgcccttagccaatgttagactcagcctaatataagaggggagaagacacttccccttgtgccaaagctggggctccctctggtagagtcactgcctccagaaggtctttggtacatacacgacctagcaatggtggagagggcaagatgggaactgaggaaaacatctttcagtaaatggccttgctcaaaagggacatgctatggctaattatgcctatcctagccctaccagaagttcagctgtaaagaatgatcacttgttaggttcagttaaaccttgttcactcctgagaactgcaattctgtgaacagaataactaaattcaggcctcagccagaaagtagaattatgacatttccatgtatttttgtgttttgagacctgcttgacagttgttcataactagaataagctaaaaatatctttgtttaaatgaatacatgttccacttaatgacagaaaagtaaattcacaaacttgctaaaaattacttctaaattgtggacaagataacctggctttgggtctctggctttagtgtaagcatccaaattgcatagtgataataatctctattgaacatagggatgcatggatagattaaatcaccctcaacactgatggacatttgaaagcaaaagaagtgtcagctgtggtccttgccatccccagtaggaggcaaggcagatcctcatagccaggagcagtgagtggcaccaagctgggagcttaacagtgaccaaggccaagtgtcagtgcaagcaggagagcacagggggagctttgagaaggcatgtgttgcatgcaccagggaagggctggtgtatctctggggataaagctgaaggatgactgggatttttctgtaatcaaagagagagaattttaaatggtattaacactgttcttgaaagaggtaaggtatgtccaatctaaaattacattgtaggagtttgtgggtgtcctgtgggtttctgttcagttgttttggtagcctcatttttcttaaatttcttttgcagttgttgaaatctataccagtgggagttggtcagatctatggctgtgataatccatggacagggggcattttcctgggagccatcctactctcctccccactcatgtgcctgcatgctgccataggatcattgctgggcatagcagcgggtgagcacaagagcccttaccaaatattgagcacctcctccatcccatgcattgcctcaggcatcttctgtgctccagatcttccttgagatcttggcttcctagggaccaatgggagttcccgggatgcttcctgctaactttcaatcccaccctcagtttccttccagaacatcctgcctttagtcctgagttctgacccctcctgtcttaacaggactcagtctttcagccccatttgaggacatctactttggactctggggtttcaacagctctctggcctgcattgcaatgggaggaatgttcatggcgctcacctggcaaacccacctcctggctcttggctgtggtgagtctcccacgcccctgggggagggctgctcatgactacaggatctcaatcaaggataagcagtaaaaacggactgcatgaaaaatcagggccagggttctggcttgagcccacttgctgtctaagtgtgtgaacaggacaagtgacgtcccctctctgagagcattaaaatcacctctgcctacctctctgatgattgtgaaggcaggagcctattgagtcatattaatatcctaaaacatggatgtttgggaggatagaaaaagaaaaatcccagttattcttcagctttatccccagagatacaccagcccttccctggtgcatgccacacatgccttctcaaagctccccctgtgctcacgggctctccagcttgcactgacacttggccttggccaccaataagctcctagaatggtggcactcactgctcctggctgtgaggatctgccatgcctcccactggggatggcaaggacctcagctgacactccttttgctttcaactgacttgtcttgcgttcttcaaactagttgtttgacccaacaaactaaacgggaataactccagctaaatacagagcaatgtcccctggtaaatcagggttgattacatttacccctttgagtgagcatcacagtaacccagccattctaaaacttcagaatgcatcagaatcacctgaaagacttgttaaaacacaaatcgctgggccccctcctcagtctgattcagcgtcagagataaggggaagaatatttctttttttatttttctaaaaaacagtctcattctgagccaagatcgcgccactgcacttcagcctgggcaacagagcaagacttcatctcaaaaaaaaaaaaaaaagagaaaagaaaaaaaaagaaaaagggtctcattctgttgcccaggctggagtgcggtggtgtgaacacagctcactgcagcctcaacctcctgggctcaagcaatcctgcagcctcagcctcccaagtaaagtagctaggaccacaggcgtgccaccatgcctggttaattttttattttttatagagatggggtctccctatgttacccaggctgatcttgaattcccgggctcaagcaatcctcccgcctccacctcccaaagtgctgggattacaggcataagccaccatgccggcagaatttccacttctaacaagttctcagggggtgctgatgctgttgctctcaggatcacatttcaagaactgctgtattaatcctttctgactcccagtgttctagccagactcagcctgtcagagcgagaaggcatcctgagacctctactccatccttcttactttactgttggggtcctgaggccagagaggctaagggatgtgccgcagggaatctggacagcaatgggtaaatccacccccggaacccacacttaccatccacctccagagttatcccaccgcactcctctgcttcccttttatagcattcaggccctcacggcaacctcttaggtgaaaacagactgcatgtgatttggatctgaaaagctaatagatcccaggtggattttgagtggaggctcattcacccatagcctctggcatgcctaattcaatcaaagtataagcatttaagataatattctagagtggagagaatgagatttgcttgggaacaaaaaggaggagggatagtgtaatgtggagaaattatgtctaatctagtggaaatatatgtctagaatcagtttatcaccagattaatcaagccaaggtatctaaacagttatgaaaacagtgggccatgtatcaggcgggtttagaatagatttctgcactggcagaaaatgggatggtaccaacggtttctaaagacccattccattttgattcgatgctatagcaagggtaacataactcaggttgctgtgatgtagccatgtagatgtcattttgtcaaattctttactattactcagctatttcacctagctgttctgttgaaatgttgaactccttctccatattcgttcacaaggataaaggagaggattacagacaggtgctgtagccacctgagttcagctgggttggaatgtttatcctacaacctttcagctttattctgagattggttaggggtttccacctgagttcagctgggttagaatgtttatcctacaacctttcagctttattctgagattggttaggggtttcaaacctttatttgggatgcatacctttatttttctggaggaagtagccacaaatatgtattaaacacacatgatacaaaagacagtaccaggaagagcaaggggtttagaagctttaggtcccatgcagttcctgcacagagtgttacaatagagggcagaagccaggcaagggagtgagcccaagaggaccatgcaatctttgtgggagaagaagaagtccatagtacaggattctccagggggccatttccactcagaattatcacaaagtacctccaggaagaagggggcttttccataaatgctagaaaataagaggaggaattctgtttggtggaaagtgtggtgcaggccagcatggggacagcctgagcatgtccttcaagatcaaggagaaggcattttgagcacaggagatggcgacgaggtttttgtttttctgggttttttgttgttttttgttttttggtttttttttttttttttttgacagagtcttgctctgttgccaggctggaatgcagtggcacagtggcacgatcttggctcactgcaacctccgactccctggttcaagcggttctcctgcctcagcctcccaagtagctgggcttacaggcacgcaccatcacgcctagctaatttttgtatttttagtagagacggggtttcaccatgttggccaggatggtctcaatcttctgacctcatgatctgtccaccccggcctcccaaagtgctgggattacaagtatgagccaccgcacctggcgggtgctgagttttttgttttatgttgttgttgttgtttgagatggactcttgctctgtagctcaggctggcatgcagtggcacgatctcagctcactgcaacctctgcctcccgggtcccggttcaagcaattcttctgcctcagcctccccagtagctgggattacaggcatgtgccaccatgcccagctaatttttttttgtatttttagtagagatggggtttcaccatgttggccaggctggtcttgaactcctgacctcgtgatccacctgccttggcctcccaaagtgctgggattacaggcgtgagccacagtgcccagctagtgatgaggttttgacagaccatggagaagaatgaagtcgaagctcttgacatgttgtttccccaaagtgggaatctttgatattttctcaattatagaagcagcacagatttattgtataaaacaaaacaaaaatgtaatctgtatagaaatgtatgaaacagaaagtggaaatactccatcttactccctagagagggcttttttgcccccttcttataaggatccttgtgattacattgggtccattcaatagtctaggaaattctctccatctcaaggtctttaacttaatcacagctgctgctaattcccttttgccatgtgaggtcacatattctcaagttctgaggtttaagatgtagacgtctttggagaccattattcttcctaccacactcaccttcctttggatagattttttttttttttaactggtgtagcataatggttgaggcagtcaactgagctaaagagctcagactctggtgccagacagcctggattcaattccagcaggtctgctacttactagcgtatttgcttatgaatgtaagcaaattacttaacctttctatgcctcagtttccccatcttagaaaatggaagttaccatatttaattcatacagttgttctgatgattaagttagttaatgcatgtctgaaactcatagaacaaatagtgtctagcactcgctcagcactatttaaaagtctggaaaaacagtttttctggtggatttgcataacttattaagaatcaagcttgtttattttctcctctcaattgcttaagtttatcaacatctgtatcttctccccaaatatgactgatacccaagcctgcctttacttcctctgagaaggcccacccctgatgactactaaaaccattgatactgtatagaatttttattttggatttgtcgtaagtataagtttttgttttgggtacttgcttatttaggcaactgtaaactttattaacttgcttattcactctgacttagttcatattaaccttctgtacttttttttttttgagacagagtctcactctgttccccaggctggagtgcagtggcacaatctcagctcactgcagcctccacctcctgggttcaagcgattcctatgcctcagactcccaagtagctgggattacagacatgcaccaccatgcccagctaattttttgtactttttgtagagacagggttttgccatgttggccaggctggtctcaaactcctgacctcaagtgatccacctgcctcggcctcccaaagtgctaggattactggtggattactttttcaaagagggtttgcaaagagagttttgttttcttcaaagagggtttgcaaagagaccttgtatgctggagaatatcttcattttaccttcatttaaattttagtttagctagctaccaaactcaagatttaacatttttttctcaatattttgaaagttgtcctcaaagactactccattgtcttcttatacccaaaattgctattaagatgtctgaaaagaaactaattcttgttaaaattgattttatttttctctctggactctctgaattttctctttgcatatgagatatatatatggttttatttcactattatctgtctagatgtaacttttttttctatgctagtaggtactcaagtcctctcaacatgagccctcatatcttcctttaattctggaaacatcatcagtttttactttgtcaaatcttttcaatttttcccctctccttctgtgatttctagtatttgagtacaatactttatgctaagtttttcataactcttgactttttcttaatattttccatctatcttttcctgaggcccttcagttcagctgattggcccgatcattctttggctctgtccattgcaccgatcacattatctgttgagttctccatttctggttcattaattaaattttactggctgggtgcagtgactcacacctgtaaacccagcactttgggaggccaaggcgggtggatcacaaggtcaagagattgagaccatcctggctaacacggtgaaaccccatctctactaaaaatacaaaaattagctgggcgtggtagcacgcgcctatagtcccagctactcaggaggctgaggcaggagaaccacttgaactcggaaggcagagctgcagtgagctgagatcatgccactgcactccagcctgggtgacagagagagactctgtctcaaagaaaaaattatcgactgtaggttgttcagtttgttgtccttcttttatggtatttgctctcctgggatgtcccctttccttgtcctgggagctcacgtttccctcgggataccagctgtttgggtgagtctctgggcagagatggaagcccaggttggagctgcatttttcctggtgcatctaaggaaaaaggggtcccctgccacagggtgtagaacctccattgctcaaggctgtggagatggtgactgtgtagacattttatatgataagtgcccttttgctgggggaagttcagattgcttctagtttgaaatcattacaaagagtcctgaaatgaatatttttggtacaaatgtccttgtgtactttgtacaagcatttctgtaagaaagaagattcaccttcttttcaagaagctaaattgatgggttaaagggaatgccaattttgatttcagtggatgccaacttcatctccaaaagagccataccagtttccactgctgccagcagtgtgtgagagtgcccactgggcccccacaaggtacaatcagacttttaaatctctgtgcatggatttttgagacagatctccagccccccttggaaagcaaatctcacatgtaaaatgccacagcaagtttcagcttgtccacatcaccctgatactgccaaacaaaagaccaaccctcttagccaacataaataagtgacagacatttattacagagctgtttttttatcagtccccagtggctttatcaggaagtggactcaggaaactctgacagaacctggcactgctgtctttctggcctctaagccagagcaactgcgtggccagagaacatctcaatgttgttgttttaccagtggagagtgtaaacatattgtgtatctcttcccaatggttgggttatcgcagtgggactcacctgtggcagtccattggaagggacactatccaggaggagctgaaatccagtttccccttcagtactcaagggccttttcttccctcagctaccaagaatgctgtcagggtcattgcctacaaactgatgatgctgtgcagaattgcgcctctactgtaaggctttcccggtcctacttggcgagtcttaattgacatacctaccattaaataatctatcacttgtactatggagagaaaagcaactttgaattggagatcacttcacagcagcataacagtatgagacgtaaacgtgccaaaagtgagccttagaagtgtaatggatattttaaaaagagagaaagcaacaaggcctcatgtgctcaggggtggtgttgtggtagagggggcactcaagagatcagggacagagggccccagtgcttggcagagggccaatgaatagttgttaaattaattgattaaatttcaacaatgaatgaaattggtgtaaccaaggagagaaacccttctaagccaagccatgagcacccttctgctcagagcagtagctcagtcccatggtgaaagagatgcatttacagctgtgtttatggaaatacaagctctcatttgagattcttcacctcccagtaaggcagatcttcaaggtgcctttttacagatgatgaaactagattccaagacagtgatttgttatacaacaaataaaatggcagagctgggatttgaaaccagtactgtttccaaagaccagcctttcccactagtgtgagacaattcatacgtgaaagaatttgatatactattgaataagaaacaccaggataaaaagacaaaatattggtaaaaggacagaagtctatggtaaagtaaatgaggatcacagagcctctcccaccatgtctgccacatccccacacaccaagatagctgacgtaccagacatgaagacgagatggtgagtgtgtctcacggtgagctccggtggcccaagtggctgtgtggccattatatgaaggtcattcttcaggctgtccccatgaaacctgagggcttccctgagcctctgtgagccttctcttcaaccaaaactgaggaatagataattagctggttgagatctttgcttttgttgttttacactgaaagtcacccatatactcgaattactgattctacaattttttggccactcaaagcaaataaaaacataagacgttggctgggcgcggtggctcatgcctgtaatcccagcactttgggaggccgagacgggcagatgacaaggtcaggagattgagaccatcctggttaacatggtgaaaccccgtctctactaacaatacaaaaaaaaaaaaattagctgggcgtagtggtgggcacctgtagtcccagctactcgggaggctgaggcaggagaatggcgtgaacccaggaggcggagcttgcagtgagcagagatcacgccagtgccctccagcctgggcgactgagtgagactccatctccaaaaaaaataaaaaataaaaaaaaagacgtttattcattgattttaatggtattggagaagatgttatcaaggggaggaatctcaagtttgtgttcagttcctgctgttctctgagttctttccttcttattttgtaaacatggttttgttttggtttttagtacacaggctgccaaagcaagcactatgattttttgtagctgtgaattcaattcattaatatgagaatcctagatgctatctcaagaaacattcataggtttcattttaattcagctatgcttggataaaacatcagagaaatttatttgccatggaaggcctttcccttaagtattagcaataacaacaaaatagtaaccataaaaaaactacctttattgagcacttactgtgtgctaaacacatgcattatttcctttcatcctcacaccaacaccatgaaaaatatattcctcttacttccattgtacaggtgaggaaatggaggcttaaaacagagcccatggagctcctaagtgatggagccaggatttgaacccaggactgctgactttaggctcatgcttgtaatcagggcactgtgcattccaggtgatttatattggaaggcagcctttcctgtgattaaaagtgcatctacgaagcattgttctttccctcctttttttttctgtagccctgttcacggcctatcttggagtcggcatggcaaactttatggctgaggtgagtttgctttagtctcacttttcattagcgtaattgaccagcttacaactatatgggaaatgctcctgaagtccactgggctggcatccagtggcaggatccatgaccatgagaagcactgctctcccttctcctggagctccctggcctttctttcagcatcacagcaaactttagtccaaaccacaatcacccagttgttacaagtatcagattgcttggtttaaaaaaaaatgaaacgtaggttgtataacatattatcaagttcagagtctaactctaagtgataagaagtagactttaggatatcttttacttaaacagaaagccagatattccattgcaggtgatgcagggccggtttctgatagcttagtccatgttgatgtggtcatggctgctaaggagtcaaggcagtatctagcccttttggcagcagcatggagattttatctgggagggtccttaaggagacacagtgtctttctggtggaaagccaaagtcccattacacacatgcatgatggagagtacatcagagcacatggggcccttcacatgtcaacaaagaagattcacaggcatcagtcccaggacccaaatgggcaagctgcacaccagagtcagctaggaagacagaaaaatatggagccttaggccctgtcctttggtatttctgatagagtaggtcttgtatgatgcttgaacatctgtgtttttttttaactcccccagatgattctgatgtgcagtcagattagggtacccctacactccatcacaccccagggaggtccatgcatcaggtcagagctaaccaatggtgtatgctcagaattgtgtgagtttccatgagcagcacaaagaggacctaccctcaaggaacttagagtctatttgggagacagaatggaaagaaacaaagcaagtcaagtctaagatctagaccaggcagaagtcaaggtcagagaggtcactgtgggctggactaatcagagaaggccttgtggacatgaagactggtcaggggccatttgcagtttgcaagtgtcatctctgtcaaatgttctcttggcacatctggtgcaggaagtctgaatatatgagagggagagaaagacatacaagatagagacataagtggctgccctaaagaatggatgtcaacattccaacaactcaatgccctgagattgtaaattcagtctccacgagcatgcacagaatccagagcaatgcccccagtggttcatccccctgggctgaatgcaagtagagggggatgccttgtgcagctcagctgtcagatgggatctgaaaggagcgtgtggctttctcttcttccccaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtgagaacaagccccatttgcagccatggtcacgagtcatttctgcctgactgctccagctaacttccagggtctcagcaaactgctgtttttcacgagtatcaactttcatactgacgcgtctgtaatctgttcttatgctcattttgtattttcctttcaactccaggaatatccttgagcatatgagagtcacatccaggtgatgtgctctggtatggaatttgaaaccccaatggggccttggcactaagactggaatgtatataaagtcaaagtgctccaacagaaggaggaagtgaaaacaaactattagtatttattgatattcttggtgtttagctggctcgatgatgttaacagtattaaaaattaaaccccataaaccaactaagccttatggaattcacagtcacaaaatcgaagttaatccagaattctgtgataagcagcttggctttttttttaaatcaatgcaagttacacattatagccagaatctgtatcacagaggtgcaagctgacagcagagctcagtccccacttcctgcaaacaatggcctgcaccctatcccttgtgtgtgtgacattctctcatgggacaatgttggggtttttcagactgacaggactgcaagagggagaaaggaattttgtcaatcaaaattattctgtattgcaacttttctcagagattgcaaaggattttttaggtagagattatttttccttatgaaaaatgatctgttttaaatgagataaaataggagaagttcctggcttaacctgttcttacatattaaagaaaagttacttactgtatttatgaaatactcagcttaggcatttttactttaacccctaaattgattttgtaaatgccacaaatgcatagaattgttaccaacctccaaagggctctttaaaatcatattttttattcatttgaggatgtcttataaagactgaaggcaaaggtcagattgcttacgggtgttatttttataagttgttgaattccttaatttaaaaaagctcattattttttgcacactcacaatattctctctcagaaatcaatggcatttgaaccaccaaaaagaaataaagggctgagtgcggtggctcacgcctgtaatcccagcactttggggagcccaggcgggcagattgcttgaacccaggagttcaagaccagcctgggcagcatggtgaaaccctgtatctacaaaaaatacaaaaattagccaggcatggtggtgggtgcctgtagttccagctacttgggaggctgaggtgggaaaatgacttgagcccaggaggaggaggctgcagtgagctaagattgcaccactgcactccaacctgggcgacaagagtgaaactgtgtctctcaaaaaaaaaaaaaaacaaacaaaaacaaaaacaaaacaaaacaaaacaaaacaaaacaggtaaggattcccctgttttcctctctttaattttaaagttatcagttccgtaaagtctctgtaaccaaacatactgaagacagcaacagaagtcacgttcagggactggctcacacctgtaatcccagcactttgggagatggaggtaaaaggatctcttgagcccaggagttcaagaccagcttgggcaacatagcaagactccatctcttaaaaaataaaaatagtaacattagccaggtgtagcagcacacatctgcagcagctactcaggaggctgaggtggaaagatcgcttgtgcacagaagttcgaggctgcagtgagctatatgatcatgtcactgcactccagcctgtgtgaccgagcaagaccctatctcaaaaaaattaattaattaattaattaattaatttaaaaaggaagtcatgttcatttactttccacttcagtgtgtatcgtgtagtattttggaggttggaaagtgaaacgtaggaatcctgaagattttttccacttctagtttgcagtgctcagtgcacaatatacattttgctgaatgaataaacagaaatagggaagtaaacctacaaatattttagggagaagctcacttcttccttttctcaggaaaccaagcaagcaaacatatcgttccaattttaaaacccagtgaccaaagcctttggaactatgaatttgcaactgtcataggtttatggatattgctgtggagaagctcaattttcagtgtttgaactgaaccctttcttgttagggaacgtgtgaaagaagaattgtggggaaaaaaaagcaagcataaccaaagatcatcagcagtgaagaatctaggctgtggctgagagaaccagaggcctctaaaatggacccgagtcgatcttcagaacagggatctaccatgcaggagcttcttgtgctcacacaaatctgtaaatgggaacattgtacattgtcgaatttaaatgatattaattttctcaagctatttttgttactattttcctaaaattgaatatttgcagggagcacttatactttttcctaatgtctgtataacaaatttctatgcaagtacatgaataaattatgctcacagctca

SEQ ID NO: 2 - DNA, 28394 bp, Homo Sapien
acacagagcagagtggggctctgagtatataactgttaggtgcctccctccagcaccatctcctgagaagcactctcccttgtcgtggaggtgggcaaatctttatcagccactgccttctgctgccaggaagccagctagagtggtgtaagtactcatccttatttctattcatttccaactattcatcatttggggcttgtcttcacagttctaagttttgctctttttcttaatgaagaaaatgttttatatcaccggaattgatcagaagtagcaaaatcagagttctggtagactagaaagcaatttaccaaagccacaggcttcttcctggaagctcaaaggcatgcctttattcgtgatttctgaagcaaggtgcatgcagcacctgagctgatgtggaagagggtttgcagggaggtgtccacccaatgtgctcaatgattctgggttaatcaacactattaggagtttcaggttgtgttcttgaaataataatttgggctgtgttcttgaaataagttcgaggcgagtgtctacaagactcaaaagaaaaaagtgggccactgggaatggccctttccagtgatggatttatggactcctctgtgtgtgctgtcatgctgaagggaatgttcttgtgcacccatcgggagaacaagtcagtcacaactgaagccacgaatttggcagcttccttgcagctgcactctctggagtctggaatcaagacttctgggagtagtgttttccaaggagggaagtgttttaaccaggacacaggaatatctgacagcattttctttgtttccaattacagctttaaagaaaactgggcatctcctgctacttaaaatcaaaaactacctaaaataaagattatagtaagtaccaaataagtgtcaatgctgaaagtctctttattatgctagaccatgagtgtttaaatgctttcttctatatccatatccaacacttcatattatttttaaaagtaatagctgaagcatggaaaattgaagacttcaggtctctccaattgcacaaatttctaatacatgctggcaatagaatatattttatttcgtgtaataaaatagaggatattagttgacctgaaatcttgatattgccttgtattaaaatgctaagcactgcttcattttactagtgatctggggtatgaaaagtgctttttgacttctgctggaaagctcttcaggtgcagcttccaggatattcttgggatgttaacttcagcacacataagccttgctgtagatgtgtcagctttgaggcacagggagacatttgtttgtcagagagtaactgcttctggcaagggcatagggtgaaactggggatagcagagctctttctttgtggttgttcaacccccaccccaagattagttcaaagtgaccgtgaagatagtctgtgcccaccgcatcgctaagtcctagccctctctgcatactccagcacacagaaactgctgcttcacttgtttgttgacttgaaccgaaccttgggtggcattaatgtgcctggcccaagactgaaaaattaagaaccaccagagctgacctattccataagacccagtctgcctgccacgtactgagtgaatctggatgatgcccactctgatccttggttttctcttctataaaatgaaggcttgaactacgtggtctctaaaatcctacctagctctcaaatttctcttggttctaggaaaatattgatgttgagctcaaggaaggggttctccaaggtgtgtgattttggtggtagaggaaaggccggtgccaggcaggggcagaaggagacgctgtctacactgagaaaatgtgacaacccctgcttgtctcttttttcattcttcattgtttcttatttctttgtttttagctttatataacatgagagccctaccactgggtttcttaaccatttgttctttatcaaataaaaatattcataatgcaacatgcaggcacatcagtgtggtacagaactagccagctagtttactataggtaaatatacacacatgcatgcacacacacaatttttacctgagacatgtcagaagtgtttcctaaaattgtggatttttctgagtcattctggtaaagggtaggttttcaggttttaggccaagccagaagaagaaagtaaaaacagaataaacaacagggggagaaaaagagaaataccacacacacaactggaacttctggtaaaagagtgatattcttggatgcaatggaagttttaaaaaggaaaaagaaaatttataaaaagctgccacatttgtggaattcaactaaaaactgtttattattaacaaagtgatgttcaaaatttaagagttcttggcctggcatgatgacttatgcctgtaatcccagtgttttgggaggctaaggtgggaggatcacttgaggccaggaattcaaaaccagcctggacaatacaatgagactttgtctctaaaaaaaaataaaataaattaaaataaacacagctggatgtggtggcacaggaaaaaaaaataccatttaggagtctcttaaaggcagcttgtgaatgcttacaaagcgtggctagtatcttattacagaaaacagagcccacatcatgcatccttcttctcacatttcataaacaaggccaagggaaactgctgtggggcaacctgttgctttggtgttggtccccaagatgcagccctcacaatctgcccccaaacgtgtcagaacatgaaccccctcctccccctctggaagaagcaacctcagatccaacagcagagacacgcagcagaacaaaatctgggcattggtccctgtgtaggatggcttcccgttattttttttttaagcaaagtaaatgaacatcaaatttccatagtcagctgctgtctttctgcccactgagagctctttggtgaaggcaaagtcctccttcttcattagcggtctcccatgtggggccacatcttccctcaccaggaacccagtgggcgcgctccagcccccctcagcttgccttttgcgtggtcattagagctagggcacacgtcatgctgattcacatatttttgccctttgtcatgtattgagaaaaagtaaggatgaatggacggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggtatgcttccttcgtgcagcctctggctcggggaacctgggagcctgctccaaactctggtgtatcttttccgggcagagcctgggaagtgggggttggctgtgagctaagccaaaggcacagggatcttggtccaaaaagccccatggcgctcaccttggtttagaggctagaccattgagctgagaagttttgacagccatggaaaagctggggataagtcacctggggttttacgtttaccctgtgtctattttattagagtgccttttacttattgtcccttcttcttagttgaaattaatggcctgcttcactggggctaagatgtttgaacattagcagaaggtcctggctgcatagccttgccttgtcttcccagttaggatgtaaggactcttaaagttccctaagaaatgcaaatattttagcatggcaaaattctaggccaactacaactgtaagtttcgtatttctcctaagtggttctcatgcctgacttctggagcaaggagtcaggtctcccaggggctctagaagggttcagctgttcagaataaatggttcctggggactctaaaatagcagcaactgtctgcccaggtcatgagaagacccctctctgcaggacatcctagccctacaacccatcccaattatgttgaaattagattcacaaatggcaataagtcttctatatgttgggctgtcgatttggagaaaactagtttaatctttacttaactttgggtggctcaacaggagactcgggccgctcaggctctcaatcacgtctggccagttctattatcaggtttcgaatctgtatctccaaaatctctgaggtgatgggatatttcaagccctctaaaataaataaatatatgctgggaattttgagaacatgaatttgtttattctgaaatggtccatgttcctgctttgggagttgatggaaaatgccacttgagtgttttcatttgatgctgccaccttagggttttatagattcagttccagaaactcaaggcatttatctctttgggctgcttgtccttgcctgagctgaagcctgatgcctcccataagttggtatggctttgaaaatgggtcactacagcagaggcatgggcttatcaagcaatatgttcagctatgaaatttgaagagggagataatctgaaaataaatgacagccaccacttagattatgaaatagaagtactttttcataagtgcttaattattcatacggttttttatctttaactatggagccaactcagctccatatggacttaattttggttcctgacctccaagattcattgcaagtcacacagatgttggtatctaacattgttttaccgagataaaatgaccttggtctggaatgcattgtataaaaagctgcttttttgtgtaaagattaatagtttggcattgtttaaaaagcagaatggttagttgggcagtgaggtaatacaattgaaatgtaattgctaccaataaatcagttacccatattgatttctttactgggattaatagaagccaaagctagagttcaactttttttaataggtataacttagtatctgttcattgctatttgttagctatggtaaatggaacaatgatggggccagaaatatccatgaggaccatttgatcacagcctggcaacacagagaagacaggctggtttctctatgtgggctttcagtgtttctttggtagtgtcttatgtggctgtggcttcaacattccacaattatgccttccagggtctgatgattttggcgtttccctgcttcccaattgacctggctgtgctgttggctgttcttgcacactcaaggtggttttgccattggcttcctccctcagcctgcctctgggattatgccactgctattcttttttatctaccatcagcacaatgaaatcatcatttttgtcttcaaggtaccaaattctggtgatattggtgctttcttgcagctacttatcatgagaagtgaatggtctcatagtgaacacagtcatggttatagtgttcatacgttccagagacatgtttcctataattatgccctgcacatttttctatcatacaatccttagattacagctctttggttttcaacagctttgtccaattccatctttcccagtttctctaccttgatgaaatatccttcttgcctggttttacatatttaaataacaaattccaaaagtaaagagtatctgaggcagtcacatgacataaggacaaattcaagccatcttggacttgcagagggtggggagaccgtgtcaacacacacaattttaaaaatttcttccctttcaatcttttaaaaacaaaactttttataaaataaaaatgtaatttaaaaaggctacctgtcttggcaagtagctgatcagcctgcattggtgagcaggccattccataacctggtttcttgctccttaattgacagcatggagctaacgtacttaatttcagctctttctacgtgatttgactcattctgttaacattaactgtttttcagtcttctcaactagactgaactccttaagtgcaagaaatacacgcttagtaaatgtttgttggaccagacactgcaccttatgaaattaaagaccagaacattctcatggtagcattacagacactgatggcaaaggtactgtgggatttgggtttggctaataagctctgtggtggtgtttcagaaggaaaatggtgctctcttagttctatggaacatagtggtccagatcttctactgtaaccaggcccaaagctggctaatctggagggctctgccttagggatacttataagctctgtccttccctcaaggagccagaggaagagatagccatggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaaggtatttatcctttcacattttggagagacaggagaagtagctttgggggaaatggtttcctggtacttctacttatacctttagttatattctccaactttttatagatctctttactcaccatttttctacttttatcttttaacctgcaaacctctccatttttttttcttatggagacagtagccagggcccagctcatattagaaggcacctggcttcatcctgtagtttcagtacttaaaacttaaatttattcctttggcttcagaatttgtacctataagcatgaaaataagtgcattagatgctttcaggagcttagattctaggaggggcagtgtgggttgagcatacagtagatagaggctttcagggatctgggtgccactaatgcaacaatgggttgagagagaaatattaaagaaatatcaaaaatgtttcacttccaggaggttttgctgattttgctcagggtgggcctgtggttgaagagtatcacttggcagcttccttagctctgctttacctcatcccttccagacaaacccgtggtgctccagttcattgactggattctccggggcatatcccaagtggtgttcgtcaacaaccccatcagtggaatcctgattctggtaggacttcttgttcagaacccctggtgggctctcactggctggctgggaacagtggtctccactctgatggccctcttgctcagccaggacaggtaggtgtaccctttcaagccttctcagctcccttctgagacacaggggctgaccagttactgtgggcaacagtgataaaaccacatccttcccaggataaacaacatttagtccacagaactgtttatatttgtttttagtcagaggtcagggaatcagttacagtctcttgctcttgatatctgaataaatggctggtctaaatgatgccagattcttgtggcattacgtgctaaccagaactaagctacaagtatttccctggagaggttctgaagggatcttctttaatgattgataaaattatttgtcgtcagcattctatttgggaaaaagtgcatatgaattcagaaaaagttttagtggcttaataacccccgttatatcttgttgctatgatgagtttaggaaactcattcttcatagacagtgcaaaggtcagctcagctcctggagaaaagaataaccatgaattccaattgagtggattctgacttaagaagccttagtgagtcttctgatatattgattagattaaaaatagcacacactttataaattgatctgtcattgaagaagtgatgagctgactctcaccagggcagtagatagctccccactagccagttcctttagggagggaaccagtattccaggtgtctgagatcaacgcataatcccaatccccagtgtggtcattacacaactaagctcttgtaacactggctgcaaattgcctaaagaggtccgtggggagagagttagcaaatgctccacttttctatcaatttcaaggagtctgatttgctccctgtagaaggggattttatagcttaggttaaactctattccaatgcatgccaagaaaaggtctcctcagtttggggatggagtctataattgtgccatactgaatattcctttatgattttgctctgatgaaacatgatcaactcattttttgtcagatattatttagaagacaagtcatttatatgtgttagtttcaaatgttttactttccttggtctgaaaagactgcattaaaatggaaattctctgttttaagtaaatatatgtcttcctgtggctttaactatggcattccacaatttgtagatgttgccattaattttccactgatcaaactcaagcattaacatctccaagtcagttgttgagaggacaagtctgcatggctctctactgtcatgtgtagtcccagtctctgagttgtacctttgcaaattgtatcacctcccatttgccctcaaggattatttaagggaaacaaagaacttttgaatagggaaccccacatttaatgttcatctggattaatgtacgtgacatcatcttgcctgttgcaatggtgcctcctggcccagttagaaacaagccaagaagcagctgtcacactatcccttaccagcccctgcagtgtggctcactggctatagcacctcctgctcgagcccagcattaggcctcacctactcacttcaccatctttactcccccatccccctacagacatcatccttgagtgacaggcccttgggaagtggatcctgtgcctttcacggtgccagacgttgccaactctcagagctgtgggaatcctgccttgtcaggtcaatcaatctaggtgcccatcaatggtggattatataaagaatatgtggtgcatatacaacacgaactactacatagccataaaaaggattgaaatcaagtcctttgcagcagcatggatgtatctggagaccaatatcctaagtgaattaatgtagtaacagaaaatcaaataccacacgttttcacttacaattaggagctaaacactgggtaaacacggacatggaaatagtagacaactgggactccaaaagaggagaggaagggaaacaagtgttgaaaacctacctatcaggtactttgttcactatttgggtgacgagttcaatagaagcccaaacctcagtcagcatcatgcaatacatctatgtaacaaacctgcacatgtaccccctcaatctaaagaaggagaagaagacggggaagaaatgagattgaatactaagcaaaaagtaacctcagaaagaactgggtgctcaacatgcacataattaaatgggatacttctccaagtaagagaaaagcaattgttcttctttgcaataactttgaaatgtgcgtttggagacaacaaaatagaagcatcaggacacaaaaatgtatactaacctggaagattaatgttgataagatcaaagacactgtgaaagtgaatttacatttcaggaatcttatatctctcaccaagaaatcaaacttaagcaacagtttcatatgctaaaagcgctcttcaagtcagaggctcttgatttaaaagaataactttccaaaggaaaggctaaaagaaaacagagcagattgccttactaaactcccctttcctctcagccactgtagacctgtctttagccgtgacacctgtagagggagtcattctctatcaggggtccccaacccctgcactggagacaggtacctgtctgtggcctgttgggaactgggccgcacagcaggaggtgagcggtgggcgagtgagcatttccacctgagctccgcctcctgtcagatcagcagaagcattagcttctcataagagtgcgaaccccattatgaactgggcatgtgagggatctaggttgcttgctccttatgagaatctaatgcctgataatctgaggtggaacagtttcatcccgaaatcatcccccattccccatccatggaaaattgtcttccatgaaacctgtccctggggccaaaaaggctggggaccactgatctaaatgcacatttatatttttatctatgtatatttcacttcatgtctttattagtttttgtacgatgcttacgtagactttgaaatacatttccaaatataatctcattttttaatatgaatatgatctggaagttactagtgttatttatgtgcaagtgcaaccaaagctcacccaggaaatgtccgtgctgtgtctcttgccccacaggtcattaatagcatctgggctctatggctacaatgccaccctggtgggagtactcatggctgtcttttcggacaagggagactatttctggtggctgttactccctgtatgtgctatgtccatgacttggtaagttacaattggttttcaaaatgcctttttgaaaaaaaaaacatggcagaaggagggaatgggagttgttatatggcagagtttcagttttgcaagatgaaatatgttctctgaatgtatagtggtgatggttgtacaacaatgtgattgtccttaatgtcattgagctgcacacttaaaaatggttagccgggtgcggtggttcttgtttgtagtccaaactattcagaaggctgagggggaaggatcacttgagcccaggagttaggggctgcagtgagctatgattgcgtcaccgcactccagttctccgaacctccttgcttgggctaagtgaggaggaggaggaggaggagaaggatggaaaggaggaggagtagcaggaggagcaggagggcaaggagaaggaggaagaggagcaggaggaggacaaacagttaaaatggtaaatttaaaattggattccagtagattctgtctattggaaacagaaacaaccattttaaaagatgtatatttccttacaaccagttatttggccttttgtctgatctggctacacatccactaatacctctcaaccagaggtggctgcacattgacacttccatggggaagggaaacagtgctgcaatgaagatacgagtgcaggtgtctttttggtagaaacacactgatgcacgtggcccccacatacacttgactcctccctcccaagactctactgtcattggtctgcggtagcgcctgggctttgggagtttctaaagcttcccagatgactctaaagtatagccaaagttgagacccacttcctccatcattgcctctcaaacttgagcaatatgagaatcacctgcagggtttgttacaccacaggcatctgctccccggccccagggtttctgatgcagtctatctggggtggggcccgagaatttgcgtttctaacgcattcccacatgatgctgggagaaccactgtgcctacgtgaattcccccttacccacctgccccccaggtctcccttagaaaaaatttttttgctgaattccttttttttcaaacccaaatccttcaaactagtttttatgttgacaatgtcttacatcctttttctggaaacaaagatttccttctttctatattgtagttaaatataaaatactaatatgcacataaataagcacagcctgctgtgggcagtgtctgcagaagggatgcccacccttactgtacccacgggtgtgtggacgaggacctacctgtagagctaaactcttcaggaagtaatttgggccctgctctgaagaataggttcgtgggaaggaggcctagcctgtaagtgctcaccacgctcccttccacaatccaggaaaatgggagttctggtctttaagtgatggctctttgattgggccaacaagtgagagcctatgagggacctcgggaccatgcagcccagccccacagtttatgggctctgaggctaaggagatgcgccttgcctaggtcatgcaatttatcaacagctcaaggacacacactctgccccaccaactgtgatatcattttcctccagctcacactacctgcatccttgaacgattgtttctcttttccaaaaataggtatattaaagaaataatatctgccaaatcagaatcagggttgcctctagtggggagggagggacataagagcaagtggagggacaaaggggactttaactatgtagataatattttattttgtatgtcataagtacttcaaaaatatttttaaaatctcaatatatagctcactctgagcaaccccagagtagaatttttcaaaagccaaataagctgagagttgattttttactttatgtaatatttactgcctctataataggatttatcccaagttttctttctgtggcaaatgtgccaacacaacacgtaaggggcctgttggcaggtgaaacaaagcccctccagagtatagcgattccgtgtgtcagcctgctttgtcacatgcacattcttttgctctgttctttttttagcccaattttctcaagtgcattgaattccatgctcagcaaatgggacctccccgtcttcaccctccctttcaacatggcgttgtcaatgtacctttcagccacaggacattacaatccattctttccagccaaactggtcatacctataactacagctccaaatatctcctggtctgacctcagtgccctggaggtaagagacactggcttctcacattcgccctggctctgcaagatacgcaatggcctcctggtcaactgtccacgggtgtcagagtctcctagatgctcaggactatggtggcctttctgccttcatcttgccatttaaagcatttgttctactccagagcattagggtctaagggattttttaaaattactatttagtcaagctgatttttctgccttttcccctaaacatctacagtgctaaccccagagtacagttccactgggagtcactctatcgtaagcttgggggtgggggtgatgggagccagcccttaaggcatgtggcctccagcctggttttaaatcttccatagtctactccctccaatcaaaaaactggatgcttactcttagagcttctgacagaacctctctattctgcttttccttatggcatagctcatagaacatctacaataatttagggttcccaagctttggtaggcatcagaatcacctggggagctttaaatacccaaacaggcttcatctcagaccctctaaatcacaatctctaagggtggggcctggaacctgttttaacaaactccccaaattgtgatgcgggccagagtttgagaaccactgtatcaaggggtgaatcctatgtatctctttaaagatggctataaagagattctgtattttttaaaacctggttaacccaaatcaaattccagctcttcctgttggtgtgtaataaatatgtttaaggtttctggattatcaagaacaagagaacacctgaaattagaagaaaaccaaagaaaccttacctttttaatgtgctctcccactgtcaggttatgaaacgcccttttgtcttctttgttgagtgatcaaaacacacgaggagctcaagtcaccttctccctagcttcttgccagaaaactaaagggagcacctggaaataattcagaaggaaaaaatcaaagattcattagaactacccatgaaaaataacagtataaaatagcattaatcgatctagaactgcactaacacaggagcctctagccccatgtggctatataaatttagatgtagattagttaaaaattgagttcctcaacctctctagccacatctcaggtgcttgatagccacacgtggctaggacccactgtattagacagcacagatacagactattccatcatctcggaaagttatcctgcacagtgctgatctggggcaggggaagccttgtccttctcactctgaatgaacagcccatcctcagcaccaaccccaaccctatggctacctgagagagagttctgcagccaagtccaaaaacaaacaaacaaacaaaaaaagcatatgccatctttgccaagttccctggtctagaaatagcaaaatgtctagacatgaagactcagcatgggctggaagaatttagagtccatcttagggtagagtcaaactcacactatggtctggtgcccttagccaatgttagactcagcctaatataagaggggagaagacacttccccttgtgccaaagctggggctccctctggtagagtcactgcctccagaaggtctttggtacatacacgacctagcaatggtggagagggcaagatgggaactgaggaaaacatctttcagtaaatggccttgctcaaaagggacatgctatggctaattatgcctatcctagccctaccagaagttcagctgtaaagaatgatcacttgttaggttcagttaaaccttgttcactcctgagaactgcaattctgtgaacagaataactaaattcaggcctcagccagaaagtagaattatgacatttccatgtatttttgtgttttgagacctgcttgacagttgttcataactagaataagctaaaaatatctttgtttaaatgaatacatgttccacttaatgacagaaaagtaaattcacaaacttgctaaaaattacttctaaattgtggacaagataacctggctttgggtctctggctttagtgtaagcatccaaattgcatagtgataataatctctattgaacatagggatgcatggatagattaaatcaccctcaacactgatggacatttgaaagcaaaagaagtgtcagctgtggtccttgccatccccagtaggaggcaaggcagatcctcatagccaggagcagtgagtggcaccaagctgggagcttaacagtgaccaaggccaagtgtcagtgcaagcaggagagcacagggggagctttgagaaggcatgtgttgcatgcaccagggaagggctggtgtatctctggggataaagctgaaggatgactgggatttttctgtaatcaaagagagagaattttaaatggtattaacactgttcttgaaagaggtaaggtatgtccaatctaaaattacattgtaggagtttgtgggtgtcctgtgggtttctgttcagttgttttggtagcctcatttttcttaaatttcttttgcagttgttgaaatctataccagtgggagttggtcagatctatggctgtgataatccatggacagggggcattttcctgggagccatcctactctcctccccactcatgtgcctgcatgctgccataggatcattgctgggcatagcagcgggtgagcacaagagcccttaccaaatattgagcacctcctccatcccatgcattgcctcaggcatcttctgtgctccagatcttccttgagatcttggcttcctagggaccaatgggagttcccgggatgcttcctgctaactttcaatcccaccctcagtttccttccagaacatcctgcctttagtcctgagttctgacccctcctgtcttaacaggactcagtctttcagccccatttgaggacatctactttggactctggggtttcaacagctctctggcctgcattgcaatgggaggaatgttcatggcgctcacctggcaaacccacctcctggctcttggctgtggtgagtctcccacgcccctgggggagggctgctcatgactacaggatctcaatcaaggataagcagtaaaaacggactgcatgaaaaatcagggccagggttctggcttgagcccacttgctgtctaagtgtgtgaacaggacaagtgacgtcccctctctgagagcattaaaatcacctctgcctacctctctgatgattgtgaaggcaggagcctattgagtcatattaatatcctaaaacatggatgtttgggaggatagaaaaagaaaaatcccagttattcttcagctttatccccagagatacaccagcccttccctggtgcatgccacacatgccttctcaaagctccccctgtgctcacgggctctccagcttgcactgacacttggccttggccaccaataagctcctagaatggtggcactcactgctcctggctgtgaggatctgccatgcctcccactggggatggcaaggacctcagctgacactccttttgctttcaactgacttgtcttgcgttcttcaaactagttgtttgacccaacaaactaaacgggaataactccagctaaatacagagcaatgtcccctggtaaatcagggttgattacatttacccctttgagtgagcatcacagtaacccagccattctaaaacttcagaatgcatcagaatcacctgaaagacttgttaaaacacaaatcgctgggccccctcctcagtctgattcagcgtcagagataaggggaagaatatttctttttttatttttctaaaaaacagtctcattctgagccaagatcgcgccactgcacttcagcctgggcaacagagcaagacttcatctcaaaaaaaaaaaaaaaagagaaaagaaaaaaaaagaaaaagggtctcattctgttgcccaggctggagtgcggtggtgtgaacacagctcactgcagcctcaacctcctgggctcaagcaatcctgcagcctcagcctcccaagtaaagtagctaggaccacaggcgtgccaccatgcctggttaattttttattttttatagagatggggtctccctatgttacccaggctgatcttgaattcccgggctcaagcaatcctcccgcctccacctcccaaagtgctgggattacaggcataagccaccatgccggcagaatttccacttctaacaagttctcagggggtgctgatgctgttgctctcaggatcacatttcaagaactgctgtattaatcctttctgactcccagtgttctagccagactcagcctgtcagagcgagaaggcatcctgagacctctactccatccttcttactttactgttggggtcctgaggccagagaggctaagggatgtgccgcagggaatctggacagcaatgggtaaatccacccccggaacccacacttaccatccacctccagagttatcccaccgcactcctctgcttcccttttatagcattcaggccctcacggcaacctcttaggtgaaaacagactgcatgtgatttggatctgaaaagctaatagatcccaggtggattttgagtggaggctcattcacccatagcctctggcatgcctaattcaatcaaagtataagcatttaagataatattctagagtggagagaatgagatttgcttgggaacaaaaaggaggagggatagtgtaatgtggagaaattatgtctaatctagtggaaatatatgtctagaatcagtttatcaccagattaatcaagccaaggtatctaaacagttatgaaaacagtgggccatgtatcaggcgggtttagaatagatttctgcactggcagaaaatgggatggtaccaacggtttctaaagacccattccattttgattcgatgctatagcaagggtaacataactcaggttgctgtgatgtagccatgtagatgtcattttgtcaaattctttactattactcagctatttcacctagctgttctgttgaaatgttgaactccttctccatattcgttcacaaggataaaggagaggattacagacaggtgctgtagccacctgagttcagctgggttggaatgtttatcctacaacctttcagctttattctgagattggttaggggtttccacctgagttcagctgggttagaatgtttatcctacaacctttcagctttattctgagattggttaggggtttcaaacctttatttgggatgcatacctttatttttctggaggaagtagccacaaatatgtattaaacacacatgatacaaaagacagtaccaggaagagcaaggggtttagaagctttaggtcccatgcagttcctgcacagagtgttacaatagagggcagaagccaggcaagggagtgagcccaagaggaccatgcaatctttgtgggagaagaagaagtccatagtacaggattctccagggggccatttccactcagaattatcacaaagtacctccaggaagaagggggcttttccataaatgctagaaaataagaggaggaattctgtttggtggaaagtgtggtgcaggccagcatggggacagcctgagcatgtccttcaagatcaaggagaaggcattttgagcacaggagatggcgacgaggtttttgtttttctgggttttttgttgttttttgttttttggtttttttttttttttttttgacagagtcttgctctgttgccaggctggaatgcagtggcacagtggcacgatcttggctcactgcaacctccgactccctggttcaagcggttctcctgcctcagcctcccaagtagctgggcttacaggcacgcaccatcacgcctagctaatttttgtatttttagtagagacggggtttcaccatgttggccaggatggtctcaatcttctgacctcatgatctgtccaccccggcctcccaaagtgctgggattacaagtatgagccaccgcacctggcgggtgctgagttttttgttttatgttgttgttgttgtttgagatggactcttgctctgtagctcaggctggcatgcagtggcacgatctcagctcactgcaacctctgcctcccgggtcccggttcaagcaattcttctgcctcagcctccccagtagctgggattacaggcatgtgccaccatgcccagctaatttttttttgtatttttagtagagatggggtttcaccatgttggccaggctggtcttgaactcctgacctcgtgatccacctgccttggcctcccaaagtgctgggattacaggcgtgagccacagtgcccagctagtgatgaggttttgacagaccatggagaagaatgaagtcgaagctcttgacatgttgtttccccaaagtgggaatctttgatattttctcaattatagaagcagcacagatttattgtataaaacaaaacaaaaatgtaatctgtatagaaatgtatgaaacagaaagtggaaatactccatcttactccctagagagggcttttttgcccccttcttataaggatccttgtgattacattgggtccattcaatagtctaggaaattctctccatctcaaggtctttaacttaatcacagctgctgctaattcccttttgccatgtgaggtcacatattctcaagttctgaggtttaagatgtagacgtctttggagaccattattcttcctaccacactcaccttcctttggatagattttttttttttttaactggtgtagcataatggttgaggcagtcaactgagctaaagagctcagactctggtgccagacagcctggattcaattccagcaggtctgctacttactagcgtatttgcttatgaatgtaagcaaattacttaacctttctatgcctcagtttccccatcttagaaaatggaagttaccatatttaattcatacagttgttctgatgattaagttagttaatgcatgtctgaaactcatagaacaaatagtgtctagcactcgctcagcactatttaaaagtctggaaaaacagtttttctggtggatttgcataacttattaagaatcaagcttgtttattttctcctctcaattgcttaagtttatcaacatctgtatcttctccccaaatatgactgatacccaagcctgcctttacttcctctgagaaggcccacccctgatgactactaaaaccattgatactgtatagaatttttattttggatttgtcgtaagtataagtttttgttttgggtacttgcttatttaggcaactgtaaactttattaacttgcttattcactctgacttagttcatattaaccttctgtacttttttttttttgagacagagtctcactctgttccccaggctggagtgcagtggcacaatctcagctcactgcagcctccacctcctgggttcaagcgattcctatgcctcagactcccaagtagctgggattacagacatgcaccaccatgcccagctaattttttgtactttttgtagagacagggttttgccatgttggccaggctggtctcaaactcctgacctcaagtgatccacctgcctcggcctcccaaagtgctaggattactggtggattactttttcaaagagggtttgcaaagagagttttgttttcttcaaagagggtttgcaaagagaccttgtatgctggagaatatcttcattttaccttcatttaaattttagtttagctagctaccaaactcaagatttaacatttttttctcaatattttgaaagttgtcctcaaagactactccattgtcttcttatacccaaaattgctattaagatgtctgaaaagaaactaattcttgttaaaattgattttatttttctctctggactctctgaattttctctttgcatatgagatatatatatggttttatttcactattatctgtctagatgtaacttttttttctatgctagtaggtactcaagtcctctcaacatgagccctcatatcttcctttaattctggaaacatcatcagtttttactttgtcaaatcttttcaatttttcccctctccttctgtgatttctagtatttgagtacaatactttatgctaagtttttcataactcttgactttttcttaatattttccatctatcttttcctgaggcccttcagttcagctgattggcccgatcattctttggctctgtccattgcaccgatcacattatctgttgagttctccatttctggttcattaattaaattttactggctgggtgcagtgactcacacctgtaaacccagcactttgggaggccaaggcgggtggatcacaaggtcaagagattgagaccatcctggctaacacggtgaaaccccatctctactaaaaatacaaaaattagctgggcgtggtagcacgcgcctatagtcccagctactcaggaggctgaggcaggagaaccacttgaactcggaaggcagagctgcagtgagctgagatcatgccactgcactccagcctgggtgacagagagagactctgtctcaaagaaaaaattatcgactgtaggttgttcagtttgttgtccttcttttatggtatttgctctcctgggatgtcccctttccttgtcctgggagctcacgtttccctcgggataccagctgtttgggtgagtctctgggcagagatggaagcccaggttggagctgcatttttcctggtgcatctaaggaaaaaggggtcccctgccacagggtgtagaacctccattgctcaaggctgtggagatggtgactgtgtagacattttatatgataagtgcccttttgctgggggaagttcagattgcttctagtttgaaatcattacaaagagtcctgaaatgaatatttttggtacaaatgtccttgtgtactttgtacaagcatttctgtaagaaagaagattcaccttcttttcaagaagctaaattgatgggttaaagggaatgccaattttgatttcagtggatgccaacttcatctccaaaagagccataccagtttccactgctgccagcagtgtgtgagagtgcccactgggcccccacaaggtacaatcagacttttaaatctctgtgcatggatttttgagacagatctccagccccccttggaaagcaaatctcacatgtaaaatgccacagcaagtttcagcttgtccacatcaccctgatactgccaaacaaaagaccaaccctcttagccaacataaataagtgacagacatttattacagagctgtttttttatcagtccccagtggctttatcaggaagtggactcaggaaactctgacagaacctggcactgctgtctttctggcctctaagccagagcaactgcgtggccagagaacatctcaatgttgttgttttaccagtggagagtgtaaacatattgtgtatctcttcccaatggttgggttatcgcagtgggactcacctgtggcagtccattggaagggacactatccaggaggagctgaaatccagtttccccttcagtactcaagggccttttcttccctcagctaccaagaatgctgtcagggtcattgcctacaaactgatgatgctgtgcagaattgcgcctctactgtaaggctttcccggtcctacttggcgagtcttaattgacatacctaccattaaataatctatcacttgtactatggagagaaaagcaactttgaattggagatcacttcacagcagcataacagtatgagacgtaaacgtgccaaaagtgagccttagaagtgtaatggatattttaaaaagagagaaagcaacaaggcctcatgtgctcaggggtggtgttgtggtagagggggcactcaagagatcagggacagagggccccagtgcttggcagagggccaatgaatagttgttaaattaattgattaaatttcaacaatgaatgaaattggtgtaaccaaggagagaaacccttctaagccaagccatgagcacccttctgctcagagcagtagctcagtcccatggtgaaagagatgcatttacagctgtgtttatggaaatacaagctctcatttgagattcttcacctcccagtaaggcagatcttcaaggtgcctttttacagatgatgaaactagattccaagacagtgatttgttatacaacaaataaaatggcagagctgggatttgaaaccagtactgtttccaaagaccagcctttcccactagtgtgagacaattcatacgtgaaagaatttgatatactattgaataagaaacaccaggataaaaagacaaaatattggtaaaaggacagaagtctatggtaaagtaaatgaggatcacagagcctctcccaccatgtctgccacatccccacacaccaagatagctgacgtaccagacatgaagacgagatggtgagtgtgtctcacggtgagctccggtggcccaagtggctgtgtggccattatatgaaggtcattcttcaggctgtccccatgaaacctgagggcttccctgagcctctgtgagccttctcttcaaccaaaactgaggaatagataattagctggttgagatctttgcttttgttgttttacactgaaagtcacccatatactcgaattactgattctacaattttttggccactcaaagcaaataaaaacataagacgttggctgggcgcggtggctcatgcctgtaatcccagcactttgggaggccgagacgggcagatgacaaggtcaggagattgagaccatcctggttaacatggtgaaaccccgtctctactaacaatacaaaaaaaaaaaaattagctgggcgtagtggtgggcacctgtagtcccagctactcgggaggctgaggcaggagaatggcgtgaacccaggaggcggagcttgcagtgagcagagatcacgccagtgccctccagcctgggcgactgagtgagactccatctccaaaaaaaataaaaaataaaaaaaaagacgtttattcattgattttaatggtattggagaagatgttatcaaggggaggaatctcaagtttgtgttcagttcctgctgttctctgagttctttccttcttattttgtaaacatggttttgttttggtttttagtacacaggctgccaaagcaagcactatgattttttgtagctgtgaattcaattcattaatatgagaatcctagatgctatctcaagaaacattcataggtttcattttaattcagctatgcttggataaaacatcagagaaatttatttgccatggaaggcctttcccttaagtattagcaataacaacaaaatagtaaccataaaaaaactacctttattgagcacttactgtgtgctaaacacatgcattatttcctttcatcctcacaccaacaccatgaaaaatatattcctcttacttccattgtacaggtgaggaaatggaggcttaaaacagagcccatggagctcctaagtgatggagccaggatttgaacccaggactgctgactttaggctcatgcttgtaatcagggcactgtgcattccaggtgatttatattggaaggcagcctttcctgtgattaaaagtgcatctacgaagcattgttctttccctcctttttttttctgtagccctgttcacggcctatcttggagtcggcatggcaaactttatggctgaggtgagtttgctttagtctcacttttcattagcgtaattgaccagcttacaactatatgggaaatgctcctgaagtccactgggctggcatccagtggcaggatccatgaccatgagaagcactgctctcccttctcctggagctccctggcctttctttcagcatcacagcaaactttagtccaaaccacaatcacccagttgttacaagtatcagattgcttggtttaaaaaaaaatgaaacgtaggttgtataacatattatcaagttcagagtctaactctaagtgataagaagtagactttaggatatcttttacttaaacagaaagccagatattccattgcaggtgatgcagggccggtttctgatagcttagtccatgttgatgtggtcatggctgctaaggagtcaaggcagtatctagcccttttggcagcagcatggagattttatctgggagggtccttaaggagacacagtgtctttctggtggaaagccaaagtcccattacacacatgcatgatggagagtacatcagagcacatggggcccttcacatgtcaacaaagaagattcacaggcatcagtcccaggacccaaatgggcaagctgcacaccagagtcagctaggaagacagaaaaatatggagccttaggccctgtcctttggtatttctgatagagtaggtcttgtatgatgcttgaacatctgtgtttttttttaactcccccagatgattctgatgtgcagtcagattagggtacccctacactccatcacaccccagggaggtccatgcatcaggtcagagctaaccaatggtgtatgctcagaattgtgtgagtttccatgagcagcacaaagaggacctaccctcaaggaacttagagtctatttgggagacagaatggaaagaaacaaagcaagtcaagtctaagatctagaccaggcagaagtcaaggtcagagaggtcactgtgggctggactaatcagagaaggccttgtggacatgaagactggtcaggggccatttgcagtttgcaagtgtcatctctgtcaaatgttctcttggcacatctggtgcaggaagtctgaatatatgagagggagagaaagacatacaagatagagacataagtggctgccctaaagaatggatgtcaacattccaacaactcaatgccctgagattgtaaattcagtctccacgagcatgcacagaatccagagcaatgcccccagtggttcatccccctgggctgaatgcaagtagagggggatgccttgtgcagctcagctgtcagatgggatctgaaaggagcgtgtggctttctcttcttccccaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtgagaacaagccccatttgcagccatggtcacgagtcatttctgcctgactgctccagctaacttccagggtctcagcaaactgctgtttttcacgagtatcaactttcatactgacgcgtctgtaatctgttcttatgctcattttgtattttcctttcaactccaggaatatccttgagcatatgagagtcacatccaggtgatgtgctctggtatggaatttgaaaccccaatggggccttggcactaagactggaatgtatataaagtcaaagtgctccaacagaaggaggaagtgaaaacaaactattagtatttattgatattcttggtgtttagctggctcgatgatgttaacagtattaaaaattaaaccccataaaccaactaagccttatggaattcacagtcacaaaatcgaagttaatccagaattctgtgataagcagcttggctttttttttaaatcaatgcaagttacacattatagccagaatctgtatcacagaggtgcaagctgacagcagagctcagtccccacttcctgcaaacaatggcctgcaccctatcccttgtgtgtgtgacattctctcatgggacaatgttggggtttttcagactgacaggactgcaagagggagaaaggaattttgtcaatcaaaattattctgtattgcaacttttctcagagattgcaaaggattttttaggtagagattatttttccttatgaaaaatgatctgttttaaatgagataaaataggagaagttcctggcttaacctgttcttacatattaaagaaaagttacttactgtatttatgaaatactcagcttaggcatttttactttaacccctaaattgattttgtaaatgccacaaatgcatagaattgttaccaacctccaaagggctctttaaaatcatattttttattcatttgaggatgtcttataaagactgaaggcaaaggtcagattgcttacgggtgttatttttataagttgttgaattccttaatttaaaaaagctcattattttttgcacactcacaatattctctctcagaaatcaatggcatttgaaccaccaaaaagaaataaagggctgagtgcggtggctcacgcctgtaatcccagcactttggggagcccaggcgggcagattgcttgaacccaggagttcaagaccagcctgggcagcatggtgaaaccctgtatctacaaaaaatacaaaaattagccaggcatggtggtgggtgcctgtagttccagctacttgggaggctgaggtgggaaaatgacttgagcccaggaggaggaggctgcagtgagctaagattgcaccactgcactccaacctgggcgacaagagtgaaactgtgtctctcaaaaaaaaaaaaaaacaaacaaaaacaaaaacaaaacaaaacaaaacaaaacaaaacaggtaaggattcccctgttttcctctctttaattttaaagttatcagttccgtaaagtctctgtaaccaaacatactgaagacagcaacagaagtcacgttcagggactggctcacacctgtaatcccagcactttgggagatggaggtaaaaggatctcttgagcccaggagttcaagaccagcttgggcaacatagcaagactccatctcttaaaaaataaaaatagtaacattagccaggtgtagcagcacacatctgcagcagctactcaggaggctgaggtggaaagatcgcttgtgcacagaagttcgaggctgcagtgagctatatgatcatgtcactgcactccagcctgtgtgaccgagcaagaccctatctcaaaaaaattaattaattaattaattaattaatttaaaaaggaagtcatgttcatttactttccacttcagtgtgtatcgtgtagtattttggaggttggaaagtgaaacgtaggaatcctgaagattttttccacttctagtttgcagtgctcagtgcacaatatacattttgctgaatgaataaacagaaatagggaagtaaacctacaaatattttagggagaagctcacttcttccttttctcaggaaaccaagcaagcaaacatatcgttccaattttaaaacccagtgaccaaagcctttggaactatgaatttgcaactgtcataggtttatggatattgctgtggagaagctcaattttcagtgtttgaactgaaccctttcttgttagggaacgtgtgaaagaagaattgtggggaaaaaaaagcaagcataaccaaagatcatcagcagtgaagaatctaggctgtggctgagagaaccagaggcctctaaaatggacccgagtcgatcttcagaacagggatctaccatgcaggagcttcttgtgctcacacaaatctgtaaatgggaacattgtacattgtcgaatttaaatgatattaattttctcaagctatttttgttactattttcctaaaattgaatatttgcagggagcacttatactttttcctaatgtctgtataacaaatttctatgcaagtacatgaataaattatgctcacagctca

SEQ ID NO: 3 - DNA, 1170 bp, Homo Sapien
auggaggacagccccacuaugguuagaguggacagccccacuaugguuaggggugaaaaccagguuucgccaugucaagggagaaggugcuuccccaaagcucuuggcuaugucaccggugacaugaaagaacuugccaaccagcuuaaagacaaacccguggugcuccaguucauugacuggauucuccggggcauaucccaagugguguucgucaacaaccccgucaguggaauccugauucugguaggacuucuuguucagaaccccuggugggcucucacuggcuggcugggaacaguggucuccacucugauggcccucuugcucagccaggacaggucauuaauagcaucugggcucuauggcuacaaugccacccuggugggaguacucauggcugucuuuucggacaagggagacuauuucugguggcuguuacucccuguaugugcuauguccaugacuugcccaauuuucucaagugcauugaauuccaugcucagcaaaugggaccuccccgucuucacccucccuuucaacauggcguugucaauguaccuuucagccacaggacauuacaauccguucuuuccagccaaacuggucauaccuauaacuacagcuccaaauaucuccuggucugaccucagugcccuggaguuguugaaaucuauaccagugggaguuggucagaucuauggcugugauaauccauggacagggggcauuuuccugggagccauccuacucuccuccccacucaugugccugcaugcugccauaggaucauugcugggcauagcagcgggacucagucuuucagccccauuugagaacaucuacuuuggacucugggguuucaacagcucucuggccugcauugcaaugggaggaauguucauggcgcucaccuggcaaacccaccuccuggcucuuggcugugcccuguucacggccuaucuuggagucggcauggcaaacuuuauggcugagguuggauugccagcuuguaccuggcccuucuguuuggccacgcuauuguuccucaucaugaccacaaaaaauuccaacaucuacaagaugccccucaguaaaguuacuuauccugaagaaaaccgcaucuucuaccugcaagccaagaaaagaaugguggaaagcccuuuguga

SEQ ID NO: 4 - DNA, 1338 bp, Homo Sapien
augaauggacggucuuugauuggcggcgcuggugacgcccgucaugguccuguuuggaaggacccuuuuggaacuaaagcuggugacgcagcgcgcagaggcaucgcccggcuaagcuuggcccuggcagaugggucgcaggaacaggagccagaggaagagauagccauggaggacagccccacuaugguuagaguggacagccccacuaugguuaggggugaaaaccagguuucgccaugucaagggagaaggugcuuccccaaagcucuuggcuaugucaccggugacaugaaagaacuugccaaccagcuuaaagacaaacccguggugcuccaguucauugacuggauucuccggggcauaucccaagugguguucgucaacaaccccgucaguggaauccuaauucugguaggacuucuuguucagaaccccuggugggcucucacuggcuggcugggaacaguggucuccacucugauggcccucuugcucagccaggacaggucauuaauagcaucugggcucuauggcuacaaugccacccuggugggaguacucauggcugucuuuucggacaagggagacuauuucugguggcuguuacucccuguaugugcuauguccaugacuugcccaauuuucucaagugcauugaauuccaugcucagcaaaugggaccuccccgucuucacccucccuuucaacauggcguugucaauguaccuuucagccacaggacauuacaauccauucuuuccagccaaacuggucauaccuauaacuacagcuccaaauaucuccuggucugaccucagugcccuggaguuguugaaaucuauaccagugggaguuggucagaucuauggcugugauaauccauggacagggggcauuuuccugggagccauccuacucuccuccccacucaugugccugcaugcugccauaggaucauugcugggcauagcagcgggacucagucuuucagccccauuugaggacaucuacuuuggacucugggguuucaacagcucucuggccugcauugcaaugggaggaauguucauggcgcucaccuggcaaacccaccuccuggcucuuggcugugcccuguucacggccuaucuuggagucggcauggcaaacuuuauggcugagguuggauugccagcuuguaccuggcccuucuguuuggccacgcuauuguuccucaucaugaccacaaaaaauuccaacaucuacaagaugccccucaguaaaguuacuuauccugaagaaaaccgcaucuucuaccugcaagccaagaaaagaaugguggaaagcccuuuguga

SEQ ID NO: 5 - DNA, 1170 bp, Homo Sapien
auggaggacagccccacuaugguuagaguggacagccccacuaugguuaggggugaaaaccagguuucgccaugucaagggagaaggugcuuccccaaagcucuuggcuaugucaccggugacaugaaagaacuugccaaccagcuuaaagacaaacccguggugcuccaguucauugacuggauucuccggggcauaucccaagugguguucgucaacaaccccaucaguggaauccugauucugguaggacuucuuguucagaaccccuggugggcucucacuggcuggcugggaacaguggucuccacucugauggcccucuugcucagccaggacaggucauuaauagcaucugggcucuauggcuacaaugccacccuggugggaguacucauggcugucuuuucggacaagggagacuauuucugguggcuguuacucccuguaugugcuauguccaugacuugcccaauuuucucaagugcauugaauuccaugcucagcaaaugggaccuccccgucuucacccucccuuucaacauggcguugucaauguaccuuucagccacaggacauuacaauccguucuuuccagccaaacuggucauaccuauaacuacagcuccaaauaucuccuggucugaccucagugcccuggaguuguugaaaucuauaccagugggaguuggucagaucuauggcugugauaauccauggacagggggcauuuuccugggagccauccuacucuccuccccacucaugugccugcaugcugccauaggaucauugcugggcauagcagcgggacucagucuuucagccccauuugagaacaucuacuuuggacucugggguuucaacagcucucuggccugcauugcaaugggaggaauguucauggcgcucaccuggcaaacccaccuccuggcucuuggcugugcccuguucacggccuaucuuggagucggcauggcaaacuuuauggcugagguuggauugccagcuuguaccuggcccuucuguuuggccacgcuauuguuccucaucaugaccacaaaaaauuccaacaucuacaagaugccccucaguaaaguuacuuauccugaagaaaaccgcaucuucuaccugcaagccaagaaaagaaugguggaaagcccuuuguga

SEQ ID NO: 6 - DNA, 1338 bp, Homo Sapien
augaauggacggucuuugauuggcggcgcuggugacgcccgucaugguccuguuuggaaggacccuuuuggaacuaaagcuggugacgcagcgcgcagaggcaucgcccggcuaagcuuggcccuggcagaugggucgcaggaacaggagccagaggaagagauagccauggaggacagccccacuaugguuagaguggacagccccacuaugguuaggggugaaaaccagguuucgccaugucaagggagaaggugcuuccccaaagcucuuggcuaugucaccggugacaugaaagaacuugccaaccagcuuaaagacaaacccguggugcuccaguucauugacuggauucuccggggcauaucccaagugguguucgucaacaaccccaucaguggaauccuaauucugguaggacuucuuguucagaaccccuggugggcucucacuggcuggcugggaacaguggucuccacucugauggcccucuugcucagccaggacaggucauuaauagcaucugggcucuauggcuacaaugccacccuggugggaguacucauggcugucuuuucggacaagggagacuauuucugguggcuguuacucccuguaugugcuauguccaugacuugcccaauuuucucaagugcauugaauuccaugcucagcaaaugggaccuccccgucuucacccucccuuucaacauggcguugucaauguaccuuucagccacaggacauuacaauccauucuuuccagccaaacuggucauaccuauaacuacagcuccaaauaucuccuggucugaccucagugcccuggaguuguugaaaucuauaccagugggaguuggucagaucuauggcugugauaauccauggacagggggcauuuuccugggagccauccuacucuccuccccacucaugugccugcaugcugccauaggaucauugcugggcauagcagcgggacucagucuuucagccccauuugaggacaucuacuuuggacucugggguuucaacagcucucuggccugcauugcaaugggaggaauguucauggcgcucaccuggcaaacccaccuccuggcucuuggcugugcccuguucacggccuaucuuggagucggcauggcaaacuuuauggcugagguuggauugccagcuuguaccuggcccuucuguuuggccacgcuauuguuccucaucaugaccacaaaaaauuccaacaucuacaagaugccccucaguaaaguuacuuauccugaagaaaaccgcaucuucuaccugcaagccaagaaaagaaugguggaaagcccuuuguga

SEQ ID NO: 7 - DNA, 1170 bp, Artificial Sequence
  Wild-type SLC14A1 cDNA 1
atggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaagacaaacccgtggtgctccagttcattgactggattctccggggcatatcccaagtggtgttcgtcaacaaccccgtcagtggaatcctgattctggtaggacttcttgttcagaacccctggtgggctctcactggctggctgggaacagtggtctccactctgatggccctcttgctcagccaggacaggtcattaatagcatctgggctctatggctacaatgccaccctggtgggagtactcatggctgtcttttcggacaagggagactatttctggtggctgttactccctgtatgtgctatgtccatgacttgcccaattttctcaagtgcattgaattccatgctcagcaaatgggacctccccgtcttcaccctccctttcaacatggcgttgtcaatgtacctttcagccacaggacattacaatccgttctttccagccaaactggtcatacctataactacagctccaaatatctcctggtctgacctcagtgccctggagttgttgaaatctataccagtgggagttggtcagatctatggctgtgataatccatggacagggggcattttcctgggagccatcctactctcctccccactcatgtgcctgcatgctgccataggatcattgctgggcatagcagcgggactcagtctttcagccccatttgagaacatctactttggactctggggtttcaacagctctctggcctgcattgcaatgggaggaatgttcatggcgctcacctggcaaacccacctcctggctcttggctgtgccctgttcacggcctatcttggagtcggcatggcaaactttatggctgaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtga

SEQ ID NO: 8 - DNA, 1338 bp, Artificial Sequence
  Wild-type SLC14A1 cDNA 2
atgaatggacggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggagccagaggaagagatagccatggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaagacaaacccgtggtgctccagttcattgactggattctccggggcatatcccaagtggtgttcgtcaacaaccccgtcagtggaatcctaattctggtaggacttcttgttcagaacccctggtgggctctcactggctggctgggaacagtggtctccactctgatggccctcttgctcagccaggacaggtcattaatagcatctgggctctatggctacaatgccaccctggtgggagtactcatggctgtcttttcggacaagggagactatttctggtggctgttactccctgtatgtgctatgtccatgacttgcccaattttctcaagtgcattgaattccatgctcagcaaatgggacctccccgtcttcaccctccctttcaacatggcgttgtcaatgtacctttcagccacaggacattacaatccattctttccagccaaactggtcatacctataactacagctccaaatatctcctggtctgacctcagtgccctggagttgttgaaatctataccagtgggagttggtcagatctatggctgtgataatccatggacagggggcattttcctgggagccatcctactctcctccccactcatgtgcctgcatgctgccataggatcattgctgggcatagcagcgggactcagtctttcagccccatttgaggacatctactttggactctggggtttcaacagctctctggcctgcattgcaatgggaggaatgttcatggcgctcacctggcaaacccacctcctggctcttggctgtgccctgttcacggcctatcttggagtcggcatggcaaactttatggctgaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtga

SEQ ID NO: 9 - DNA, 1170 bp, Artificial Sequence
  Variant SLC14A1 (Val76Ile) cDNA
atggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaagacaaacccgtggtgctccagttcattgactggattctccggggcatatcccaagtggtgttcgtcaacaaccccatcagtggaatcctgattctggtaggacttcttgttcagaacccctggtgggctctcactggctggctgggaacagtggtctccactctgatggccctcttgctcagccaggacaggtcattaatagcatctgggctctatggctacaatgccaccctggtgggagtactcatggctgtcttttcggacaagggagactatttctggtggctgttactccctgtatgtgctatgtccatgacttgcccaattttctcaagtgcattgaattccatgctcagcaaatgggacctccccgtcttcaccctccctttcaacatggcgttgtcaatgtacctttcagccacaggacattacaatccgttctttccagccaaactggtcatacctataactacagctccaaatatctcctggtctgacctcagtgccctggagttgttgaaatctataccagtgggagttggtcagatctatggctgtgataatccatggacagggggcattttcctgggagccatcctactctcctccccactcatgtgcctgcatgctgccataggatcattgctgggcatagcagcgggactcagtctttcagccccatttgagaacatctactttggactctggggtttcaacagctctctggcctgcattgcaatgggaggaatgttcatggcgctcacctggcaaacccacctcctggctcttggctgtgccctgttcacggcctatcttggagtcggcatggcaaactttatggctgaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtga

SEQ ID NO: 10 - DNA, 1338 bp, Artificial Sequence
  Variant SLC14A1 (Val132Ile) cDNA
atgaatggacggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggagccagaggaagagatagccatggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaagacaaacccgtggtgctccagttcattgactggattctccggggcatatcccaagtggtgttcgtcaacaaccccatcagtggaatcctaattctggtaggacttcttgttcagaacccctggtgggctctcactggctggctgggaacagtggtctccactctgatggccctcttgctcagccaggacaggtcattaatagcatctgggctctatggctacaatgccaccctggtgggagtactcatggctgtcttttcggacaagggagactatttctggtggctgttactccctgtatgtgctatgtccatgacttgcccaattttctcaagtgcattgaattccatgctcagcaaatgggacctccccgtcttcaccctccctttcaacatggcgttgtcaatgtacctttcagccacaggacattacaatccattctttccagccaaactggtcatacctataactacagctccaaatatctcctggtctgacctcagtgccctggagttgttgaaatctataccagtgggagttggtcagatctatggctgtgataatccatggacagggggcattttcctgggagccatcctactctcctccccactcatgtgcctgcatgctgccataggatcattgctgggcatagcagcgggactcagtctttcagccccatttgaggacatctactttggactctggggtttcaacagctctctggcctgcattgcaatgggaggaatgttcatggcgctcacctggcaaacccacctcctggctcttggctgtgccctgttcacggcctatcttggagtcggcatggcaaactttatggctgaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtga

SEQ ID NO: 11 - protein, 389 aa, Homo Sapien
MEDSPTMVRVDSPTMVRGENQVSPCQGRRCFPKALGYVTGDMKELANQLKDKPVVLQFIDWILRGISQVVFVNNPVSGILILVGLLVQNPWWALTGWLGTVVSTLMALLLSQDRSLIASGLYGYNATLVGVLMAVFSDKGDYFWWLLLPVCAMSMTCPIFSSALNSMLSKWDLPVFTLPFNMALSMYLSATGHYNPFFPAKLVIPITTAPNISWSDLSALELLKSIPVGVGQIYGCDNPWTGGIFLGAILLSSPLMCLHAAIGSLLGIAAGLSLSAPFEDIYFGLWGFNSSLACIAMGGMFMALTWQTHLLALGCALFTAYLGVGMANFMAEVGLPACTWPFCLATLLFLIMTTKNSNIYKMPLSKVTYPEENRIFYLQAKKRMVESPL

SEQ ID NO: 12 - protein, 445 aa, Homo Sapien
MNGRSLIGGAGDARHGPVWKDPFGTKAGDAARRGIARLSLALADGSQEQEPEEEIAMEDSPTMVRVDSPTMVRGENQVSPCQGRRCFPKALGYVTGDMKELANQLKDKPVVLQFIDWILRGISQVVFVNNPVSGILILVGLLVQNPWWALTGWLGTVVSTLMALLLSQDRSLIASGLYGYNATLVGVLMAVFSDKGDYFWWLLLPVCAMSMTCPIFSSALNSMLSKWDLPVFTLPFNMALSMYLSATGHYNPFFPAKLVIPITTAPNISWSDLSALELLKSIPVGVGQIYGCDNPWTGGIFLGAILLSSPLMCLHAAIGSLLGIAAGLSLSAPFEDIYFGLWGFNSSLACIAMGGMFMALTWQTHLLALGCALFTAYLGVGMANFMAEVGLPACTWPFCLATLLFLIMTTKNSNIYKMPLSKVTYPEENRIFYLQAKKRMVESPL

SEQ ID NO: 13 - protein, 389 aa, Homo Sapien
MEDSPTMVRVDSPTMVRGENQVSPCQGRRCFPKALGYVTGDMKELANQLKDKPVVLQFIDWILRGISQVVFVNNPISGILILVGLLVQNPWWALTGWLGTVVSTLMALLLSQDRSLIASGLYGYNATLVGVLMAVFSDKGDYFWWLLLPVCAMSMTCPIFSSALNSMLSKWDLPVFTLPFNMALSMYLSATGHYNPFFPAKLVIPITTAPNISWSDLSALELLKSIPVGVGQIYGCDNPWTGGIFLGAILLSSPLMCLHAAIGSLLGIAAGLSLSAPFEDIYFGLWGFNSSLACIAMGGMFMALTWQTHLLALGCALFTAYLGVGMANFMAEVGLPACTWPFCLATLLFLIMTTKNSNIYKMPLSKVTYPEENRIFYLQAKKRMVESPL

SEQ ID NO: 14 - protein, 445 aa, Homo Sapien
MNGRSLIGGAGDARHGPVWKDPFGTKAGDAARRGIARLSLALADGSQEQEPEEEIAMEDSPTMVRVDSPTMVRGENQVSPCQGRRCFPKALGYVTGDMKELANQLKDKPVVLQFIDWILRGISQVVFVNNPISGILILVGLLVQNPWWALTGWLGTVVSTLMALLLSQDRSLIASGLYGYNATLVGVLMAVFSDKGDYFWWLLLPVCAMSMTCPIFSSALNSMLSKWDLPVFTLPFNMALSMYLSATGHYNPFFPAKLVIPITTAPNISWSDLSALELLKSIPVGVGQIYGCDNPWTGGIFLGAILLSSPLMCLHAAIGSLLGIAAGLSLSAPFEDIYFGLWGFNSSLACIAMGGMFMALTWQTHLLALGCALFTAYLGVGMANFMAEVGLPACTWPFCLATLLFLIMTTKNSNIYKMPLSKVTYPEENRIFYLQAKKRMVESPL

(19) United States
(12) Patent Application Publication
(10) Pub. No.: US 2019/0071683 A1
(43) Pub. Date: Mar. 7, 2019

(54) Solute Carrier Family 14 Member 1 (SLC14A1) Variants And Uses Thereof

(71) Applicant: Regeneron Pharmaceuticals, Inc., Tarrytown, NY (US)

(72) Inventors: Tanya Teslovich Dostal, Tarrytown, NY (US); Joshua Backman, Tarrytown, NY (US)

(21) Appl. No.: 16/123,373

(22) Filed: Sep. 6, 2018

Related U.S. Application Data
(60) Provisional application No. 62/555,440, filed on Sep. 7, 2017.

(51) Int. Cl. C12N 15/79; C12Q 1/6876; C12Q 1/6837

(52) U.S. Cl. CPC C12N 15/79; C12Q 1/6876; C12Q 1/6837; C12Q 2600/16; C12Q 2600/118; C12Q 2600/112; C12Q 2600/166

(57) ABSTRACT

The disclosure provides nucleic acid molecules, including cDNA, comprising an alteration that encodes variant human Solute Carrier Family 14 Member 1 (SLC14A1) proteins that associate with protection against coronary artery disease (CAD). The disclosure also provides methods for classifying subjects at risk of developing a coagulation condition, based on the identification of such alterations.

### CROSS-REFERENCE TO RELATED APPLICATIONS

This application claims priority to U.S. Provisional Application Ser. No. 62/555,440 filed Sep. 7, 2017, which is incorporated herein by reference in its entirety.

### REFERENCE TO A SEQUENCE LISTING

This application includes a Sequence Listing submitted electronically as a text file named 18923800901SEQ, created on Sep. 6, 2018, with a size of 101 kilobytes. The Sequence Listing is incorporated by reference herein.

### FIELD

The disclosure relates generally to the field of genetics. More particularly, the disclosure relates to gene alterations and polypeptide variants in the Solute Carrier Family 14 Member 1 (SLC14A1) that associate with, for example, protection against coronary artery disease (CAD).

### BACKGROUND

Various references, including patents, patent applications, accession numbers, technical articles, and scholarly articles are cited throughout the specification. Each reference is incorporated by reference herein, in its entirety and for all purposes.

Coronary artery disease (CAD) develops when the coronary arteries that supply the heart with blood, oxygen and nutrients become damaged or diseased. Common causes of CAD are cholesterol-containing deposits (plaque) and inflammation. Plaque build-up causes the coronary arteries to narrow, thus resulting in decreased blood flow to the heart. In some instances, the decreased blood flow may cause chest pain (angina), shortness of breath, or other coronary artery disease signs and symptoms. A complete blockage can cause a myocardial infarction.

Venous thromboembolism (VTE), consisting of deep venous thrombosis (DVT) and pulmonary embolism, is a recurrent and debilitating disease characterized by the formation of blood clots in veins. Family-based studies suggest that genetic variation is a major contributor to VTE risk. However, VTE has a complex etiology, and polymorphisms identified through GWAS account for about 5% of the heritable component of VTE, providing limited insight into genetic underpinnings of the disease. The identification of novel genetic variants that influence VTE risk may illuminate new therapeutic targets and guide the way to safer and more effective alternatives to current therapies for VTE prophylaxis and treatment.

### SUMMARY

The disclosure provides SLC14A1 variants that will aid in understanding the biology of SLC14A1, and will facilitate the diagnosis and treatment of coagulation conditions and CAD. The disclosure provides nucleic acid molecules (i.e., genomic DNA, mRNA, and cDNA) encoding SLC14A1 variant polypeptides, and SLC14A1 variant polypeptides, that have been demonstrated herein to be associated with protection from coagulation disorders and CAD.

The disclosure also provides isolated nucleic acid molecules comprising a nucleic acid sequence encoding a human SLC14A1 protein, wherein the protein comprises an isoleucine at the position corresponding to position 76 according to SEQ ID NO:13, or the complement of the nucleic acid sequence, or wherein the protein comprises an isoleucine at the position corresponding to position 132 according to SEQ ID NO:14, or the complement of the nucleic acid sequence.

The disclosure also provides genomic DNA molecules comprising a nucleic acid sequence encoding at least a portion of a human SLC14A1 protein, wherein the protein comprises an isoleucine at the position corresponding to position 76 according to SEQ ID NO:13, or the complement of the nucleic acid sequence, or wherein the protein comprises an isoleucine at the position corresponding to position 132 according to SEQ ID NO:14, or the complement of the nucleic acid sequence.

The disclosure also provides mRNA molecules comprising a nucleic acid sequence encoding at least a portion of a human SLC14A1 protein, wherein the protein comprises an isoleucine at the position corresponding to position 76 according to SEQ ID NO:13, or the complement of the nucleic acid sequence, or wherein the protein comprises an isoleucine at the position corresponding to position 132 according to SEQ ID NO:14, or the complement of the nucleic acid sequence.

The disclosure also provides cDNA molecules comprising a nucleic acid sequence encoding at least a portion of a human SLC14A1 protein, wherein the protein comprises an isoleucine at the position corresponding to position 76 according to SEQ ID NO:13, or the complement of the nucleic acid sequence, or wherein the protein comprises an isoleucine at the position corresponding to position 132 according to SEQ ID NO:14, or the complement of the nucleic acid sequence.

The disclosure also provides vectors comprising any of the isolated nucleic acid molecules disclosed herein.

The disclosure also provides compositions comprising any of the isolated nucleic acid molecules or vectors disclosed herein and a carrier.

The disclosure also provides host cells comprising any of the isolated nucleic acid molecules or vectors disclosed herein.

The disclosure also provides isolated or recombinant polypeptides comprising at least a portion of the human SLC14A1 protein, wherein the protein comprises an isoleucine at the position corresponding to position 76 according to SEQ ID NO:13, or the complement of the nucleic acid sequence, or wherein the protein comprises an isoleucine at the position corresponding to position 132 according to SEQ ID NO:14, or the complement of the nucleic acid sequence.

The disclosure also provides compositions comprising any of the isolated or recombinant polypeptides disclosed herein and a carrier.

The disclosure also provides a probe or a primer comprising a nucleic acid sequence comprising at least about 5 nucleotides, which hybridizes to a nucleic acid sequence encoding a human SLC14A1 protein, wherein the protein comprises an isoleucine at the position corresponding to position 76 according to SEQ ID NO:13 or wherein the protein comprises an isoleucine at the position corresponding to position 132 according to SEQ ID NO:14, or which hybridizes to the complement of the nucleic acid sequence encoding the human SLC14A1 protein, wherein the protein comprises an isoleucine at the position corresponding to position 76 according to SEQ ID NO:13 or wherein the protein comprises an isoleucine at the position corresponding to position 132 according to SEQ ID NO:14.

The disclosure also provides supports comprising a substrate to which any of the probes disclosed herein hybridize.

The disclosure also provides an alteration-specific probe or primer comprising a nucleic acid sequence which is complementary to a nucleic acid sequence encoding an SLC14A1 protein comprising an isoleucine at the position corresponding to position 76 according to SEQ ID NO:13 or comprising an isoleucine at the position corresponding to position 132 according to SEQ ID NO:14, wherein the alteration-specific probe or primer comprises a nucleic acid sequence which is complementary to a portion of the nucleic acid molecule encoding position 76 according to SEQ ID NO:13 or encoding position 132 according to SEQ ID NO:14. In some embodiments, the alteration-specific probe or primer specifically hybridizes to a portion of the nucleic acid molecule encoding a position corresponding to position 76 according to SEQ ID NO:13 or specifically hybridizes to a portion of the nucleic acid molecule encoding a position corresponding to position 132 according to SEQ ID NO:14, or to the complement of at least one of these nucleic acid molecules. The alteration-specific probe or primer does not hybridize to a nucleic acid molecule having a nucleic acid sequence encoding a wild-type SLC14A1 protein.

The disclosure also provides methods for identifying a human subject having a coagulation condition or a risk for developing a coagulation condition, or coronary artery disease or a risk for developing coronary artery disease, wherein the method comprises detecting in a sample obtained from the subject the presence or absence of a variant SLC14A1 protein comprising an isoleucine at the position corresponding to position 76 according to SEQ ID NO:13 or comprising an isoleucine at the position corresponding to position 132 according to SEQ ID NO:14; and/or a nucleic acid molecule encoding a variant SLC14A1 protein comprising an isoleucine at the position corresponding to position 76 according to SEQ ID NO:13 or comprising an isoleucine at the position corresponding to position 132 according to SEQ ID NO:14; wherein the absence of the variant SLC14A1 protein and/or the nucleic acid molecule encoding the variant SLC14A1 protein indicates that the subject has a coagulation condition or a risk for developing a coagulation condition, or coronary artery disease or a risk for developing coronary artery disease.

The disclosure also provides methods for diagnosing a coagulation condition, detecting a risk of developing a coagulation condition, coronary artery disease, or a risk for developing coronary artery disease in a human subject, comprising: detecting the presence or absence of an alteration in a nucleic acid molecule encoding an SLC14A1 protein obtained from the human subject, wherein the alteration encodes an SLC14A1 protein comprising an isoleucine at the position corresponding to position 76 according to SEQ ID NO:13 or comprising an isoleucine at the position corresponding to position 132 according to SEQ ID NO:14; and diagnosing the human subject with a coagulation condition or coronary artery disease if the subject lacks the alteration and has one or more symptoms of a coagulation condition or coronary artery disease, or diagnosing the human subject as at risk for developing a coagulation condition or coronary artery disease if the subject lacks the alteration and does not have one or more symptoms of a coagulation condition or coronary artery disease.

The disclosure also provides methods for treating a coagulation condition patient with a therapeutic agent that prevents, treats, or inhibits the coagulation condition, comprising the steps of: determining whether the patient has one or more genetic variants associated with the coagulation condition by performing or having performed a genotype assay on a DNA sample obtained from the patient to determine if the patient has one or more genetic variants associated with the coagulation condition; and when the patient has one or more of the genetic variants associated with the coagulation condition, administering to the patient the therapeutic agent that prevents, treats, or inhibits the coagulation condition.

The disclosure also provides methods for treating a coagulation condition patient with a therapeutic agent that prevents, treats, or inhibits the coagulation condition, comprising the steps of: determining whether the patient has one or more genetic variants associated with the coagulation condition by performing or having performed an assay on a protein sample obtained from the patient to determine if the patient has one or more genetic variants associated with the coagulation condition; and when the patient has one or more of the genetic variants associated with the coagulation condition, administering to the patient the therapeutic agent that prevents, treats, or inhibits the coagulation condition.

The disclosure also provides methods for treating a coronary artery disease (CAD) patient with a therapeutic agent that prevents, treats, or inhibits the coronary artery disease, comprising the steps of: determining whether the patient has one or more genetic variants associated with the coronary artery disease by performing or having performed a genotype assay on a DNA sample obtained from the patient to determine if the patient has one or more genetic variants associated with the coronary artery disease; and when the patient has one or more of the genetic variants associated with the coronary artery disease, administering to the patient the therapeutic agent that prevents, treats, or inhibits the coronary artery disease.

The disclosure also provides methods for treating a coronary artery disease (CAD) patient with a therapeutic agent that prevents, treats, or inhibits the coronary artery disease, comprising the steps of: determining whether the patient has one or more genetic variants associated with the coronary artery disease by performing or having performed an assay on a protein sample obtained from the patient to determine if the patient has one or more genetic variants associated with the coronary artery disease; and when the patient has one or more of the genetic variants associated with the coronary artery disease, administering to the patient the therapeutic agent that prevents, treats, or inhibits the coronary artery disease.

The disclosure also provides inhibitors of coagulation for use in the treatment of a coagulation condition in a human subject having an SLC14A1 protein that does not comprise an isoleucine at the position corresponding to position 76 according to SEQ ID NO:13 or that does not comprise an isoleucine at the position corresponding to position 132 according to SEQ ID NO:14.

The disclosure also provides agents for use in the treatment of CAD in a human subject having an SLC14A1 protein that does not comprise an isoleucine at the position corresponding to position 76 according to SEQ ID NO:13 or that does not comprise an isoleucine at the position corresponding to position 132 according to SEQ ID NO:14.

### BRIEF DESCRIPTION OF THE FIGURES

The accompanying figures, which are incorporated in and constitute a part of this specification, illustrate several aspects and together with the description serve to explain the principles of the disclosure.

FIG. 1 shows graphical results of a genetic association study for activated partial thromboplastin time (aPTT).

FIG. 2 shows a novel association with aPTT in the analysis.

FIG. 3 shows a Forest plot of aPTT meta-analysis for SLC14A1 Va176Ile.

FIG. 4 shows a regional plot for SLC14A1 Va1761Ile meta-analysis association with aPTT.

FIG. 5 shows a forest plot of CAD meta-analysis for SLC14A1 V76I.

FIG. 6 shows a novel association with aPTT in the analysis.

Additional advantages of the disclosure will be set forth in part in the description which follows, and in part will be apparent from the description, or can be learned by practice of the embodiments disclosed herein. The advantages of the disclosure will be realized and attained by means of the elements and combinations particularly pointed out in the appended claims. It is to be understood that both the foregoing general description and the following detailed description are exemplary and explanatory only and are not restrictive of the embodiments, as claimed.

### DESCRIPTION

Various terms relating to aspects of disclosure are used throughout the specification and claims. Such terms are to be given their ordinary meaning in the art, unless otherwise indicated. Other specifically defined terms are to be construed in a manner consistent with the definition provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the terms “subject” and “patient” are used interchangeably. A subject may include any animal, including mammals. Mammals include, without limitation, farm animals (e.g., horse, cow, pig), companion animals (e.g., dog, cat), laboratory animals (e.g., mouse, rat, rabbits), and non-human primates. In some embodiments, the subject is a human being.

As used herein, a “nucleic acid,” a “nucleic acid molecule,” a “nucleic acid sequence,” “polynucleotide,” or “oligonucleotide” can comprise a polymeric form of nucleotides of any length, may comprise DNA and/or RNA, and can be single-stranded, double-stranded, or multiple stranded. One strand of a nucleic acid also refers to its complement.

As used herein, the phrase “corresponding to” or grammatical variations thereof when used in the context of the numbering of a given amino acid or nucleic acid sequence or position refers to the numbering of a specified reference sequence when the given amino acid or nucleic acid sequence is compared to the reference sequence (e.g., with the reference sequence herein being the nucleic acid molecule or polypeptide of (wild type or full length) SLC14A1). In other words, the residue (e.g., amino acid or nucleotide) number or residue (e.g., amino acid or nucleotide) position of a given polymer is designated with respect to the reference sequence rather than by the actual numerical position of the residue within the given amino acid or nucleic acid sequence. For example, a given amino acid sequence can be aligned to a reference sequence by introducing gaps to optimize residue matches between the two sequences. In these cases, although the gaps are present, the numbering of the residue in the given amino acid or nucleic acid sequence is made with respect to the reference sequence to which it has been aligned.

For example, the phrase “a human SLC14A1 protein, wherein the protein comprises an isoleucine at the position corresponding to position 76 according to SEQ ID NO:13” (and similar phrases) means that, if the amino acid sequence of the SLC14A1 protein is aligned to the sequence of SEQ ID NO:13, the SLC14A1 protein possesses an isoleucine at the position that corresponds to position 76 of SEQ ID NO: 13. Herein, such a protein is also referred to as “a variant SLC14A1 protein” or “SLC14A1 Va176Ile.”

An SLC14A1 protein comprising an isoleucine at the position corresponding to position 76 according to SEQ ID NO:13 can easily be identified by performing a sequence alignment between the given SLC14A1 protein and the amino acid sequence of SEQ ID NO:13. Likewise, an SLC14A1 protein comprising an isoleucine at the position corresponding to position 132 according to SEQ ID NO:14 can easily be identified by performing a sequence alignment between the given SLC14A1 protein and the amino acid sequence of SEQ ID NO:14. A variety of computational algorithms exist that can be used for performing a sequence alignment in order to identify an isoleucine at a position that corresponds to position 76 in SEQ ID NO:13, or to identify an isoleucine at a position that corresponds to position 132 according to SEQ ID NO:14. For example, by using the NCBI BLAST algorithm (Altschul et al., 1997, Nuc. Acids Res., 25, 3389-3402) or CLUSTALW software (Sievers et al., 2014, Methods Mol. Biol., 1079, 105-116) sequence alignments may be performed. However, sequences can also be aligned manually.

It has been observed in accordance with the disclosure that particular variations in SLC14A1 may associate with prolonged bleeding time (e.g., diminished blood coagulation) and may serve to protect against coronary artery disease. It is believed that these variations in SLC14A1 may further provide protection against coagulation conditions. It is believed that no variants of the SLC14A1 gene or protein have any previous known association with such a protective function relating to coronary artery disease in human beings. A rare variant in the SLC14A1 gene segregating with the phenotype of protection against coronary artery disease in affected family members has been identified in accordance with the disclosure. Such protective alterations in the SLC14A1 nucleic acid result in an SLC14A1 protein with loss of function or an SLC14A1 hypomorph (e.g., partial loss of function) protein. For example, a genetic alteration that results in the replacement of a valine with an isoleucine at a position corresponding to position 76 according to SEQ ID NO:13 has been observed to indicate that the human having such an alteration may possess a protection against developing coronary artery disease or may have a lowered risk of developing coronary artery disease.

Altogether, the genetic analyses described herein surprisingly indicate that variants in the SLC14A1 gene that result in SLC14A1 proteins having loss of function or partial loss of function are associated with decreased susceptibility to coronary artery disease, and are believed to be associated with decreased susceptibility to coagulation-based events in the body. Therefore, human subjects that do not possess the SLC14A1 alteration that associates with a protection against a coagulation condition or coronary artery disease may be treated such that a coagulation condition or coronary artery disease is inhibited, the symptoms thereof are reduced, and/or development of symptoms is repressed. Accordingly, the disclosure provides isolated or recombinant SLC14A1 variant nucleic acid molecules, such as genes, mRA, and cDNA, as well as isolated or recombinant SLC14A1 variant polypeptides. Additionally, the disclosure provides methods for leveraging the identification of such variants in subjects to identify or stratify risk in such subjects of developing a coagulation condition or coronary artery disease, or to diagnose subjects as having a coagulation condition or coronary artery disease, such that subjects at risk or subjects with active disease may be treated.

The amino acid sequences for two wild type SLC14A1 proteins are set forth in SEQ ID NO:11 and SEQ ID NO:12. The wild type SLC14A1 protein having SEQ ID NO:11 is 389 amino acids in length, whereas the wild type SLC14A1 protein having SEQ ID NO:12 is 445 amino acids in length. SEQ ID NO:11 comprises a valine at position 76 and SEQ ID NO:12 comprises a valine at position 132.

The disclosure provides nucleic acid molecules encoding SLC14A1 variant proteins that associate with protection against a coagulation condition or coronary artery disease. For example, the disclosure provides isolated nucleic acid molecules comprising a nucleic acid sequence encoding a variant SLC14A1 protein, wherein the variant SLC14A1 protein is a loss of function protein or a partial loss of function protein. In particular, the disclosure provides isolated nucleic acid molecules comprising a nucleic acid sequence encoding a human SLC14A1 protein, wherein the protein comprises an isoleucine at a position corresponding to position 76 according to SEQ ID NO:13, or the complement of the nucleic acid sequence.

In some embodiments, the nucleic acid molecule comprises or consists of a nucleic acid sequence that encodes a human SLC14A1 protein having an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprises an isoleucine at a position corresponding to position 76 according to SEQ ID NO:13, or the complement of the nucleic acid sequence. In some embodiments, the nucleic acid molecule does not encode SEQ ID NO:13. Herein, if reference is made to percent sequence identity, the higher percentages of sequence identity are preferred over the lower ones.

In some embodiments, the disclosure provides isolated nucleic acid molecules comprising a nucleic acid sequence encoding a human SLC14A1 protein, wherein the protein comprises an isoleucine at a position corresponding to position 132 according to SEQ ID NO:14, or the complement of the nucleic acid sequence.

In some embodiments, the nucleic acid molecule comprises or consists of a nucleic acid sequence that encodes a human SLC14A1 protein having an amino acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprises an isoleucine at a position corresponding to position 132 according to SEQ ID NO:14, or the complement of the nucleic acid sequence. In some embodiments, the nucleic acid molecule does not encode SEQ ID NO:14. Herein, if reference is made to percent sequence identity, the higher percentages of sequence identity are preferred over the lower ones.

The nucleic acid sequence of a wild type SLC14A1 genomic DNA is set forth in SEQ ID NO:1. The wild type SLC14A1 genomic DNA comprising SEQ ID NO:1 is 28,394 nucleotides in length. Referring to SEQ ID NO:1, position 6963 of the wild type SLC14A1 genomic DNA is a guanine.

The disclosure provides genomic DNA molecules encoding a variant SLC14A1 protein. In some embodiments, the genomic DNA molecules encode variant SLC14A1 proteins that are loss of function proteins or partial loss of function proteins. In some embodiments, the variant SLC14A1 genomic DNA comprises or consists of a nucleic acid sequence encoding an SLC14A1 protein comprising an isoleucine at the position corresponding to position 76 according to SEQ ID NO:13 or comprising an isoleucine at the position corresponding to position 132 according to SEQ ID NO:14. In some embodiments, the variant SLC14A1 genomic DNA comprises or consists of a nucleic acid sequence encoding an SLC14A1 protein comprising an isoleucine at the position corresponding to position 76 according to SEQ ID NO:13. In some embodiments, the variant SLC14A1 genomic DNA comprises or consists of a nucleic acid sequence encoding an SLC14A1 protein comprising an isoleucine at the position corresponding to position 132 according to SEQ ID NO:14.

In some embodiments, the variant SLC14A1 genomic DNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13, and comprises an isoleucine at the position corresponding to position 76 according to SEQ ID NO:13. In some embodiments, the variant SLC14A1 genomic DNA comprises or consists of a nucleic acid sequence encoding a variant SLC14A1 protein having SEQ ID NO:13. In some embodiments, the variant SLC14A1 genomic DNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13, and comprises an isoleucine at the position corresponding to position 76 according to SEQ ID NO:13, provided that the variant SLC14A1 genomic DNA does not comprises or consists of a nucleic acid sequence that encodes SEQ ID NO:13.

In some embodiments, the variant SLC14A1 genomic DNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14, and comprises an isoleucine at the position corresponding to position 132 according to SEQ ID NO:14. In some embodiments, the variant SLC14A1 genomic DNA comprises or consists of a nucleic acid sequence encoding a variant SLC14A1 protein having SEQ ID NO:14. In some embodiments, the variant SLC14A1 genomic DNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13, and comprises an isoleucine at the position corresponding to position 76 according to SEQ ID NO:13, provided that the variant SLC14A1 genomic DNA does not comprises or consists of a nucleic acid sequence that encodes SEQ ID NO:14.

In some embodiments, the variant SLC14A1 genomic DNA comprises or consists of a nucleic acid sequence comprising an adenine at a position corresponding to position 6963 according to SEQ ID NO:2. In contrast, the wild type SLC14A1 genomic DNA comprises a guanine at a position corresponding to position 6963 according to SEQ ID NO:1. In some embodiments, the genomic DNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:2, and comprises an adenine at a position corresponding to position 6963 according to SEQ ID NO:2. In some embodiments, the genomic DNA comprises or consists of a nucleic acid sequence according to SEQ ID NO:2. In some embodiments, the genomic DNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:2, and comprises an adenine at a position corresponding to position 6963 according to SEQ ID NO:2, provided that the genomic DNA does not comprise or consist of a nucleic acid sequence according to SEQ ID NO:2.

In some embodiments, the variant SLC14A1 genomic DNA comprises a nucleic acid sequence which is at least about 90%, at least about 95%, at least about 96%, at least about 97%, at least about 98%, or at least about 99% identical to SEQ ID NO:2, provided that the nucleic acid sequence comprises a codon at the position corresponding to positions 6963 to 6965 according to SEQ ID NO:2 that encodes an isoleucine, or the complement thereof. In some embodiments, the variant SLC14A1 genomic DNA comprises the nucleotides corresponding to positions 6963 to 6965 according to SEQ ID NO:2. In some embodiments, the variant SLC14A1 genomic DNA comprises SEQ ID NO:2. In some embodiments, the variant SLC14A1 genomic DNA comprises a nucleic acid sequence which is at least about 90%, at least about 95%, at least about 96%, at least about 97%, at least about 98%, or at least about 99% identical to SEQ ID NO:2, provided that the nucleic acid sequence comprises a codon at the position corresponding to positions 6963 to 6965 according to SEQ ID NO:2 that encodes an isoleucine, and provided that the variant SLC14A1 genomic DNA does not comprise SEQ ID NO:2, or the complement thereof.

In some embodiments, the isolated nucleic acid molecules comprise less than the entire genomic DNA sequence. In some embodiments, the isolated nucleic acid molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10000, at least about 11000, at least about 12000, at least about 13000, at least about 14000, at least about 15000, at least about 16000, at least about 17000, at least about 18000, at least about 19000, at least about 20000, at least about 21000, at least about 22000, at least about 23000, at least about 24000, at least about 25000, at least about 26000, at least about 27000, or at least about 28000 contiguous nucleotides of SEQ ID NO:2. In some embodiments, the isolated nucleic acid molecules comprise or consist of at least about 1000 to at least about 2000 contiguous nucleotides of SEQ ID NO:2.

In some embodiments, the isolated nucleic acid molecules comprise less than the entire genomic DNA sequence. In some embodiments, the isolated nucleic acid molecules comprise or consist of at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or at least about 3000 contiguous nucleotides of SEQ ID NO:2. In some embodiments, such contiguous nucleotides may be combined with other nucleic acid molecules of contiguous nucleotides to produce the cDNA molecules described herein.

Such isolated nucleic acid molecules can be used, for example, to express variant SLC14A1 mRNAs and proteins or as exogenous donor sequences. It is understood that gene sequences within a population can vary due to polymorphisms, such as SNPs. The examples provided herein are only exemplary sequences, and other sequences are also possible.

In some embodiments, the isolated nucleic acid molecules comprise a variant SLC14A1 minigene, in which one or more nonessential segments encoding SEQ ID NO:13 or SEQ ID NO:14 have been deleted with respect to the corresponding wild type SLC14A1 genomic DNA. In some embodiments, the deleted nonessential segment(s) comprise one or more intron sequences. In some embodiments, the SLC14A1 minigene has at least about 70%, at least about 75%, at least about 80%, at least about 85%, at least about 90%, at least about 95%, at least about 96%, at least about 97%, at least about 98%, at least about 99%, or 100% sequence identity to a portion of SEQ ID NO:13 or SEQ ID NO:14, wherein the minigene comprises a nucleic acid sequence having an adenine at a position corresponding to position 6963 according to SEQ ID NO:2.

The nucleic acid sequences of two wild type SLC14A1 mRNAs are set forth in SEQ ID NO:3 and SEQ ID NO:4. The wild type SLC14A1 mRNA comprising SEQ ID NO:3 is 1170 nucleotides in length. Referring to SEQ ID NO:3, position 226 of the wild type SLC14A1 mRNA is a guanine. The wild type SLC14A1 mRNA comprising SEQ ID NO:4 is 1338 nucleotides in length. Referring to SEQ ID NO:4, position 394 of the wild type SLC14A1 mRNA is a guanine.

The disclosure also provides mRNA molecules encoding variant SLC14A1 proteins. In some embodiments, the mRNA molecules encode variant SLC14A1 proteins that are loss of function proteins or partial loss of function proteins. In some embodiments, the variant SLC14A1 mRNA comprises or consists of a nucleic acid sequence encoding an SLC14A1 protein comprising an isoleucine at the position corresponding to position 76 according to SEQ ID NO:13 or comprising an isoleucine at the position corresponding to position 132 according to SEQ ID NO:14. In some embodiments, the variant SLC14A1 mRNA comprises or consists of a nucleic acid sequence encoding an SLC14A1 protein comprising an isoleucine at the position corresponding to position 76 according to SEQ ID NO:13. In some embodiments, the variant SLC14A1 mRNA comprises or consists of a nucleic acid sequence encoding an SLC14A1 protein comprising an isoleucine at the position corresponding to position 132 according to SEQ ID NO:14.

In some embodiments, the variant SLC14A1 mRNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13, and comprises an isoleucine at the position corresponding to position 76 according to SEQ ID NO:13. In some embodiments, the variant SLC14A1 mRNA comprises or consists of a nucleic acid sequence encoding a variant SLC14A1 protein having SEQ ID NO:13. In some embodiments, the variant SLC14A1 mRNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13, and comprises an isoleucine at the position corresponding to position 76 according to SEQ ID NO:13, provided that the variant SLC14A1 mRNA does not comprise or consist of a nucleic acid sequence that encodes SEQ ID NO:13.

In some embodiments, the variant SLC14A1 mRNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14, and comprises an isoleucine at the position corresponding to position 132 according to SEQ ID NO:14. In some embodiments, the variant SLC14A1 mRNA comprises or consists of a nucleic acid sequence encoding a variant SLC14A1 protein having SEQ ID NO:14. In some embodiments, the variant SLC14A1 mRNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13, and comprises an isoleucine at the position corresponding to position 76 according to SEQ ID NO:13, provided that the variant SLC14A1 mRNA does not comprise or consist of a nucleic acid sequence that encodes SEQ ID NO:14.

In some embodiments, the variant SLC14A1 mRNA comprises or consists of a nucleic acid sequence comprising an adenine at a position corresponding to position 226 according to SEQ ID NO:5. In contrast, the wild type SLC14A1 mRNA comprises a guanine at a position corresponding to position 226 according to SEQ ID NO:5. In some embodiments, the variant SLC14A1 mRNA comprises or consists of a nucleic acid sequence comprising the codon AUC at positions corresponding to positions 226 to 228 according to SEQ ID NO:5. In contrast, the wild type SLC14A1 mRNA comprises the codon GUC at positions corresponding to positions 226 to 228 according to SEQ ID NO:5. In some embodiments, the variant SLC14A1 mRNA does not comprise or consist of a nucleic acid sequence according to SEQ ID NO:5.

In some embodiments, the variant SLC14A1 mRNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:5, and comprises an adenine at a position corresponding to position 226 according to SEQ ID NO:5. In some embodiments, the variant SLC14A1 mRNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:5, and comprises an adenine at a position corresponding to position 226 according to SEQ ID NO:5, provided that the variant SLC14A1 mRNA does not comprise or consist of a nucleic acid sequence according to SEQ ID NO:5.

In some embodiments, the variant SLC14A1 mRNA comprises or consists of a nucleic acid sequence which is at least about 90%, at least about 95%, at least about 96%, at least about 97%, at least about 98%, or at least about 99% identical to SEQ ID NO:5, provided that the nucleic acid sequence encodes an amino acid sequence which comprises an isoleucine at the position corresponding to position 76 according to SEQ ID NO:13, or the complement thereof. In some embodiments, the variant SLC14A1 mRNA comprises or consists of a nucleic acid sequence according to SEQ ID NO:5. In some embodiments, the variant SLC14A1 mRNA comprises or consists of a nucleic acid sequence which is at least about 90%, at least about 95%, at least about 96%, at least about 97%, at least about 98%, or at least about 99% identical to SEQ ID NO:5, provided that the nucleic acid sequence encodes an amino acid sequence which comprises an isoleucine at the position corresponding to position 76 according to SEQ ID NO:13, or the complement thereof, and provided that the variant SLC14A1 mRNA does not comprise or consist of a nucleic acid sequence according to SEQ ID NO:5, or the complement thereof.

In some embodiments, the variant SLC14A1 mRNA comprises or consists of a nucleic acid sequence comprising an adenine at a position corresponding to position 394 according to SEQ ID NO:6. In contrast, the wild type SLC14A1 mRNA comprises a guanine at a position corresponding to position 394 according to SEQ ID NO:6. In some embodiments, the variant SLC14A1 mRNA comprises or consists of a nucleic acid sequence comprising the codon AUC at positions corresponding to positions 394 to 396 according to SEQ ID NO:6. In contrast, the wild type SLC14A1 mRNA comprises the codon GUC at positions corresponding to positions 394 to 396 according to SEQ ID NO:6. In some embodiments, the variant SLC14A1 mRNA does not comprise or consist of a nucleic acid sequence according to SEQ ID NO:6.

In some embodiments, the variant SLC14A1 mRNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:6, and comprises an adenine at a position corresponding to position 394 according to SEQ ID NO:6. In some embodiments, the variant SLC14A1 mRNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:6, and comprises an adenine at a position corresponding to position 394 according to SEQ ID NO:6, provided that the variant SLC14A1 mRNA does not comprise or consist of a nucleic acid sequence according to SEQ ID NO:6.

In some embodiments, the variant SLC14A1 mRNA comprises a nucleic acid sequence which is at least about 90%, at least about 95%, at least about 96%, at least about 97%, at least about 98%, or at least about 99% identical to SEQ ID NO:6, provided that the nucleic acid sequence encodes an amino acid sequence which comprises an isoleucine at the position corresponding to position 132 according to SEQ ID NO:14, or the complement thereof. In some embodiments, the variant SLC14A1 mRNA comprises or consists of a nucleic acid sequence according to SEQ ID NO:6. In some embodiments, the variant SLC14A1 mRNA comprises a nucleic acid sequence which is at least about 90%, at least about 95%, at least about 96%, at least about 97%, at least about 98%, or at least about 99% identical to SEQ ID NO:6, provided that the nucleic acid sequence encodes an amino acid sequence which comprises an isoleucine at the position corresponding to position 132 according to SEQ ID NO:14, or the complement thereof, provided that the variant SLC14A1 mRNA does not comprise a nucleic acid sequence according to SEQ ID NO:6.

In some embodiments, the isolated nucleic acid molecule comprises less nucleotides than the entire SLC14A1 mRNA sequence. In some embodiments, the isolated nucleic acid molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1100, or at least about 1200 contiguous nucleotides of SEQ ID NO:5. In some embodiments, the isolated nucleic acid molecules comprise or consist of at least about 200 to at least about 500 contiguous nucleotides of SEQ ID NO:5. In this regard, the longer mRNA molecules are preferred over the shorter ones. In some embodiments, the isolated nucleic acid molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:5. In this regard, the longer mRNA molecules are preferred over the shorter ones. In some embodiments, such mRNA molecules include the codon that encodes the isoleucine at the position that corresponds to position 76 according to SEQ ID NO:13. In some embodiments, such mRNA molecules include the adenine at the position corresponding to position 226 according to SEQ ID NO:5. In some embodiments, such mRNA molecules include the codon AUC at positions corresponding to positions 226 to 228 according to SEQ ID NO:5.

In some embodiments, the isolated nucleic acid molecule comprises less nucleotides than the entire SLC14A1 mRNA sequence. In some embodiments, the isolated nucleic acid molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1100, at least about 1200, or at least about 1300 contiguous nucleotides of SEQ ID NO:6. In some embodiments, the isolated nucleic acid molecules comprise or consist of at least about 200 to at least about 500 contiguous nucleotides of SEQ ID NO:6. In this regard, the longer mRNA molecules are preferred over the shorter ones. In some embodiments, the isolated nucleic acid molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:6. In this regard, the longer mRNA molecules are preferred over the shorter ones. In some embodiments, such mRNA molecules include the codon that encodes the isoleucine at the position that corresponds to position 132 according to SEQ ID NO:14. In some embodiments, such mRNA molecules include the adenine at the position corresponding to position 394 according to SEQ ID NO:6. In some embodiments, such mRNA molecules include the codon AUC at positions corresponding to positions 394 to 396 according to SEQ ID NO:6.

The nucleic acid sequence of two wild type SLC14A1 cDNAs are set forth in SEQ ID NO:7 and SEQ ID NO:8. The wild type SLC14A1 cDNA comprising SEQ ID NO:7 is 1173 nucleotides in length, including the stop codon. Referring to SEQ ID NO:7, position 226 of the wild type SLC14A1 cDNA is a guanine. The wild type SLC14A1 cDNA comprising SEQ ID NO:8 is 1341 nucleotides in length, including the stop codon. Referring to SEQ ID NO:8, position 394 of the wild type SLC14A1 cDNA is a guanine.

The disclosure also provides variant SLC14A1 cDNA molecules encoding a variant SLC14A1 protein. In some embodiments, the variant cDNA molecules encode variant SLC14A1 proteins that are loss of function proteins or partial loss of function proteins. In some embodiments, the variant SLC14A1 cDNA comprises or consists of a nucleic acid sequence encoding an SLC14A1 protein comprising an isoleucine at the position corresponding to position 76 according to SEQ ID NO:13 or comprising an isoleucine at the position corresponding to position 132 according to SEQ ID NO:14. In some embodiments, the variant SLC14A1 cDNA comprises or consists of a nucleic acid sequence encoding an SLC14A1 protein comprising an isoleucine at the position corresponding to position 76 according to SEQ ID NO:13. In some embodiments, the variant SLC14A1 cDNA comprises or consists of a nucleic acid sequence encoding an SLC14A1 protein comprising an isoleucine at the position corresponding to position 132 according to SEQ ID NO:14. In some embodiments, the variant SLC14A1 cDNA does not comprise or consist of a nucleic acid sequence encoding a variant SLC14A1 protein according to SEQ ID NO:13 or SEQ ID NO:14.

In some embodiments, the variant SLC14A1 cDNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprises an isoleucine at the position corresponding to position 76 according to SEQ ID NO:13. In some embodiments, the variant SLC14A1 cDNA comprises or consists of a nucleic acid sequence encoding a variant SLC14A1 protein having SEQ ID NO:13. In some embodiments, the variant SLC14A1 cDNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprises an isoleucine at the position corresponding to position 76 according to SEQ ID NO:13, provided that the variant SLC14A1 cDNA does not comprise or consist of a nucleic acid sequence according to SEQ ID NO:13.

In some embodiments, the variant SLC14A1 cDNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprises an isoleucine at the position corresponding to position 132 according to SEQ ID NO:14. In some embodiments, the variant SLC14A1 cDNA comprises or consists of a nucleic acid sequence encoding a variant SLC14A1 protein having SEQ ID NO:14. In some embodiments, the variant SLC14A1 cDNA comprises or consists of a nucleic acid sequence that encodes a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprises an isoleucine at the position corresponding to position 132 according to SEQ ID NO:14, provided that the variant SLC14A1 cDNA does not comprise or consist of a nucleic acid sequence according to SEQ ID NO:14.

In some embodiments, the variant SLC14A1 cDNA comprises or consists of a nucleic acid sequence comprising an adenine at a position corresponding to position 226 according to SEQ ID NO:9. In contrast, the wild type SLC14A1 cDNA comprises a guanine at a position corresponding to position 226 according to SEQ ID NO:9. In some embodiments, the variant SLC14A1 cDNA comprises or consists of a nucleic acid sequence comprising the codon AUC at positions corresponding to positions 226 to 228 according to SEQ ID NO:9. In contrast, the wild type SLC14A1 cDNA comprises the codon GUC at positions corresponding to positions 226 to 228 according to SEQ ID NO:9. In some embodiments, the variant SLC14A1 cDNA does not comprise or consist of a nucleic acid sequence according to SEQ ID NO:9.

In some embodiments, the variant SLC14A1 cDNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:9 and comprises an adenine at a position corresponding to position 226 according to SEQ ID NO:9. In some embodiments, the variant SLC14A1 cDNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:9 and comprises an adenine at a position corresponding to position 226 according to SEQ ID NO:9, provided that the variant SLC14A1 cDNA does not comprise or consist of a nucleic acid sequence according to SEQ ID NO:9.

In some embodiments, the variant SLC14A1 cDNA comprises or consists of a nucleic acid sequence which is at least about 90%, at least about 95%, at least about 96%, at least about 97%, at least about 98%, or at least about 99% identical to SEQ ID NO:9, provided that the nucleic acid sequence encodes an isoleucine at the position corresponding to position 76 according to SEQ ID NO:13, or the complement thereof. In some embodiments, the variant SLC14A1 cDNA comprises or consists of a nucleic acid sequence according to SEQ ID NO:9. In some embodiments, the variant SLC14A1 cDNA comprises or consists of a nucleic acid sequence which is at least about 90%, at least about 95%, at least about 96%, at least about 97%, at least about 98%, or at least about 99% identical to SEQ ID NO:9, provided that the nucleic acid sequence encodes an isoleucine at the position corresponding to position 76 according to SEQ ID NO:13, or the complement thereof, provided that the variant SLC14A1 cDNA does not comprise or consist of a nucleic acid sequence according to SEQ ID NO:9.

In some embodiments, the variant SLC14A1 cDNA comprises or consists of a nucleic acid sequence comprising an adenine at a position corresponding to position 394 according to SEQ ID NO:10. In contrast, the wild type SLC14A1 cDNA comprises a guanine at a position corresponding to position 394 according to SEQ ID NO:10. In some embodiments, the variant SLC14A1 cDNA comprises or consists of a nucleic acid sequence comprising the codon AUC at positions corresponding to positions 394 to 396 according to SEQ ID NO:10. In contrast, the wild type SLC14A1 cDNA comprises the codon GUC at positions corresponding to positions 394 to 296 according to SEQ ID NO:10. In some embodiments, the variant SLC14A1 cDNA does not comprises or consists of a nucleic acid sequence according to SEQ ID NO:10.

In some embodiments, the variant SLC14A1 cDNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:10 and comprises an adenine at a position corresponding to position 394 according to SEQ ID NO:10. In some embodiments, the variant SLC14A1 cDNA comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:10 and comprises an adenine at a position corresponding to position 394 according to SEQ ID NO:10, provided that the variant SLC14A1 cDNA does not comprise or consist of a nucleic acid sequence according to SEQ ID NO:10.

In some embodiments, the variant SLC14A1 cDNA comprises or consists of a nucleic acid sequence which is at least about 90%, at least about 95%, at least about 96%, at least about 97%, at least about 98%, or at least about 99% identical to SEQ ID NO:10, provided that the nucleic acid sequence encodes an isoleucine at the position corresponding to position 132 according to SEQ ID NO:10, or the complement thereof. In some embodiments, the variant SLC14A1 cDNA comprises or consists of a nucleic acid sequence according to SEQ ID NO:10. In some embodiments, the variant SLC14A1 cDNA comprises or consists of a nucleic acid sequence which is at least about 90%, at least about 95%, at least about 96%, at least about 97%, at least about 98%, or at least about 99% identical to SEQ ID NO:10, provided that the nucleic acid sequence encodes an isoleucine at the position corresponding to position 132 according to SEQ ID NO:10, or the complement thereof, provided that the variant SLC14A1 cDNA does not comprise or consist of a nucleic acid sequence according to SEQ ID NO:10.

In some embodiments, the isolated nucleic acid molecule comprises less nucleotides than the entire SLC14A1 cDNA sequence. In some embodiments, the isolated nucleic acid molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1100, or at least about 1200 contiguous nucleotides of SEQ ID NO:9. In some embodiments, the isolated nucleic acid molecules comprise or consist of at least about 200 to at least about 500 contiguous nucleotides of SEQ ID NO:9. In this regard, the longer cDNA molecules are preferred over the shorter ones. In some embodiments, the isolated nucleic acid molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:9. In this regard, the longer cDNA molecules are preferred over the shorter ones. In some embodiments, such cDNA molecules include the codon that encodes the isoleucine at the position that corresponds to position 76 according to SEQ ID NO:13. In some embodiments, such cDNA molecules include the adenine at the position corresponding to position 226 according to SEQ ID NO:9. In some embodiments, such cDNA molecules include the codon AUC at positions corresponding to positions 226 to 228 according to SEQ ID NO:9.

In some embodiments, the isolated nucleic acid molecule comprises less nucleotides than the entire SLC14A1 cDNA sequence. In some embodiments, the isolated nucleic acid molecules comprise or consist of at least about 5, at least about 8, at least about 10, at least about 12, at least about 15, at least about 20, at least about 25, at least about 30, at least about 35, at least about 40, at least about 45, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 1100, at least about 1200, or at least about 1300 contiguous nucleotides of SEQ ID NO:10. In some embodiments, the isolated nucleic acid molecules comprise or consist of at least about 200 to at least about 500 contiguous nucleotides of SEQ ID NO:10. In this regard, the longer cDNA molecules are preferred over the shorter ones. In some embodiments, the isolated nucleic acid molecules comprise or consist of at least about 50, at least about 60, at least about 70, at least about 80, at least about 90, at least about 100, at least about 200, at least about 300, at least about 400, or at least about 500 contiguous nucleotides of SEQ ID NO:10. In this regard, the longer cDNA molecules are preferred over the shorter ones. In some embodiments, such cDNA molecules include the codon that encodes the isoleucine at the position that corresponds to position 132 according to SEQ ID NO:14. In some embodiments, such cDNA molecules include the adenine at the position corresponding to position 394 according to SEQ ID NO:10. In some embodiments, such cDNA molecules include the codon AUC at positions corresponding to positions 394 to 396 according to SEQ ID NO:10.

The disclosure also provides isolated nucleic acid molecules that hybridize to variant SLC14A1 genomic DNA (such as SEQ ID NO:2), variant SLC14A1 minigenes, variant SLC14A1 mRNA (such as SEQ ID NO:5 and/or SEQ ID NO:6), and/or variant SLC14A1 cDNA (such as SEQ ID NO:9 and/or SEQ ID NO:10). In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, at least about 10000, at least about 11000, or at least about 1200 nucleotides. In some embodiments, the isolated nucleic acid molecule comprises or consists of at least 15 nucleotides. In some embodiments, the isolated nucleic acid molecule comprises or consists of at least 15 nucleotides to at least about 35 nucleotides. In some embodiments, such isolated nucleic acid molecules hybridize to variant SLC14A1 genomic DNA (such as SEQ ID NO:2), variant SLC14A1 minigenes, variant SLC14A1 mRNA (such as SEQ ID NO:5 and/or SEQ ID NO:6), and/or variant SLC14A1 cDNA (such as SEQ ID NO:9 and/or SEQ ID NO:10) under stringent conditions. Such nucleic acid molecules may be used, for example, as probes, as primers, or as alteration-specific probes or primers as described or exemplified herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to variant SLC14A1 genomic DNA (such as SEQ ID NO:2), variant SLC14A1 minigenes, variant SLC14A1 mRNA (such as SEQ ID NO:5 and/or SEQ ID NO:6), and/or variant SLC14A1 cDNA (such as SEQ ID NO:9 and/or SEQ ID NO:10). In some embodiments, the isolated nucleic acid molecules comprise or consist of from about 15 to about 100 nucleotides, or from about 15 to about 35 nucleotides. In some embodiments, the isolated nucleic acid molecules comprise or consist of from about 15 to about 100 nucleotides. In some embodiments, the isolated nucleic acid molecules comprise or consist of from about 15 to about 35 nucleotides.

In some embodiments, any of the nucleic acid molecules, genomic DNA molecules, cDNA molecules, or mRNA molecules disclosed herein can be purified, e.g., are at least about 90% pure. In some embodiments, any of the nucleic acid molecules, genomic DNA molecules, cDNA molecules, or mRNA molecules disclosed herein can be purified, e.g., are at least about 95% pure. In some embodiments, any of the nucleic acid molecules, genomic DNA molecules, cDNA molecules, or mRNA molecules disclosed herein can be purified, e.g., are at least about 99% pure. Purification is according to the hands of a human being, with human-made purification techniques.

The disclosure also provides fragments of any of the isolated nucleic acid molecules, genomic DNA molecules, cDNA molecules, or mRNA molecules disclosed herein. In some embodiments, the fragments comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 contiguous residues of any of the nucleic acid sequences disclosed herein, or any complement thereof. In this regard, the longer fragments are preferred over the shorter ones. In some embodiments, the fragments comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 contiguous residues. In this regard, the longer fragments are preferred over the shorter ones. In some embodiments, the fragments comprise or consist of at least about 20, at least about 25, at least about 30, or at least about 35 contiguous residues. In some embodiments, the fragments comprise or consist of at least about 20 contiguous residues. In some embodiments, the fragments comprise or consist of at least about 25 contiguous residues. In some embodiments, the fragments comprise or consist of at least about 30 contiguous residues. In some embodiments, the fragments comprise or consist of at least about 35 contiguous residues. It is envisaged that the fragments comprise of consist of the portion of the nucleic acid molecule that encodes an isoleucine at a position corresponding to position 76 according to SEQ ID NO:13, or that encodes an isoleucine at a position corresponding to position 132 according to SEQ ID NO:14. Such fragments may be used, for example, as probes, as primers, or as allele-specific primers as described or exemplified herein.

The disclosure also provides probes and primers. The probe or primer of the disclosure have a nucleic acid sequence that specifically hybridizes to any of the nucleic acid molecules disclosed herein, or the complement thereof. In some embodiments, the probe or primer specifically hybridizes to any of the nucleic acid molecules disclosed herein under stringent conditions. The disclosure also provides nucleic acid molecules having nucleic acid sequences that hybridize under moderate conditions to any of the nucleic acid molecules disclosed herein, or the complement thereof. A probe or primer according to the disclosure preferably encompasses the nucleic acid codon which encodes the isoleucine at a position corresponding to position 76 according to SEQ ID NO:13, or the complement thereof. A probe or primer according to the disclosure preferably encompasses the nucleic acid codon which encodes the isoleucine at a position corresponding to position 132 according to SEQ ID NO:14, or the complement thereof. Thus, in a preferred embodiment, the disclosure provides alteration-specific primers which are defined herein above and below in more detail.

A probe according to the disclosure may be used to detect the variant SLC14A1 nucleic acid molecule (e.g., genomic DNA, mRNA, and/or cDNA) encoding the variant SLC14A1 protein (e.g., according to SEQ ID NO:13 and/or SEQ ID NO:14). In addition, a primer according to the disclosure may be used to amplify a nucleic acid molecule encoding a variant SLC14A1 protein, or fragment thereof. The disclosure also provides a pair of primers comprising one of the primers described above.

The nucleic acid molecules disclosed herein can comprise a nucleic acid sequence of a naturally occurring SLC14A1 genomic DNA, cDNA, or mRNA transcript, or can comprise a non-naturally occurring sequence. In some embodiments, the naturally occurring sequence can differ from the non-naturally occurring sequence due to synonymous mutations or mutations that do not affect the encoded SLC14A1 polypeptide. For example, the sequence can be identical with the exception of synonymous mutations or mutations that do not affect the encoded SLC14A1 polypeptide. A synonymous mutation or substitution is the substitution of one nucleotide for another in an exon of a gene coding for a protein such that the produced amino acid sequence is not modified. This is possible because of the degeneracy of the genetic code, with some amino acids being coded for by more than one three-base pair codon. Synonymous substitutions are used, for example, in the process of codon optimization. The nucleic acid molecules disclosed herein can be codon optimized.

Also provided herein are functional polynucleotides that can interact with the disclosed nucleic acid molecules. Functional polynucleotides are nucleic acid molecules that have a specific function, such as binding a target molecule or catalyzing a specific reaction. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

Antisense molecules are designed to interact with a target nucleic acid molecule through either canonical or non-canonical base pairing. The interaction of the antisense molecule and the target molecule is designed to promote the destruction of the target molecule through, for example, RNase-H-mediated RNA-DNA hybrid degradation. Alternately, the antisense molecule is designed to interrupt a processing function that normally would take place on the target molecule, such as transcription or replication. Antisense molecules can be designed based on the sequence of the target molecule. Numerous methods for optimization of antisense efficiency by identifying the most accessible regions of the target molecule exist. Exemplary methods include, but are not limited to, in vitro selection experiments and DNA modification studies using DMS and DEPC. Antisense molecules generally bind the target molecule with a dissociation constant (k_{d}) less than or equal to about 10⁻⁶, less than or equal to about 10⁻⁸, less than or equal to about 10⁻¹⁰, or less than or equal to about 10⁻¹². A representative sample of methods and techniques which aid in the design and use of antisense molecules can be found in the following non-limiting list of U.S. Pat. Nos. 5,135,917; 5,294,533; 5,627,158; 5,641,754; 5,691,317; 5,780,607; 5,786,138; 5,849,903; 5,856,103; 5,919,772; 5,955,590; 5,990,088; 5,994,320; 5,998,602; 6,005,095; 6,007,995; 6,013,522; 6,017,898; 6,018,042; 6,025,198; 6,033,910; 6,040,296; 6,046,004; 6,046,319; and 6,057,437. Examples of antisense molecules include, but are not limited to, antisense RNAs, small interfering RNAs (siRNAs), and short hairpin RNAs (shRNAs).

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be in a vector or exogenous donor sequence comprising the isolated nucleic acid molecule and a heterologous nucleic acid sequence. The isolated nucleic acid molecules can also be linked or fused to a heterologous label, such as a fluorescent label. Other examples of labels are disclosed elsewhere herein.

The label can be directly detectable (e.g., fluorophore) or indirectly detectable (e.g., hapten, enzyme, or fluorophore quencher). Such labels can be detectable by spectroscopic, photochemical, biochemical, immunochemical, or chemical means. Such labels include, for example, radiolabels that can be measured with radiation-counting devices; pigments, dyes or other chromogens that can be visually observed or measured with a spectrophotometer; spin labels that can be measured with a spin label analyzer; and fluorescent labels (e.g., fluorophores), where the output signal is generated by the excitation of a suitable molecular adduct and that can be visualized by excitation with light that is absorbed by the dye or can be measured with standard fluorometers or imaging systems. The label can also be, for example, a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term “label” can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels are known and include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain nucleotide analogs such as, for example, 5-substituted pyrimidines, 6-azapyrimidines, and N-2, N-6 and O-6 substituted purines including, but not limited to, 2-aminopropyladenine, 5-propynyluracil, 5-propynylcytosine, and 5-methylcytosine can increase the stability of duplex formation. Often, base modifications can be combined with, for example, a sugar modification, such as 2′-O-methoxyethyl, to achieve unique properties such as increased duplex stability.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included.

Nucleotide substitutes include molecules having similar functional properties to nucleotides, but which do not contain a phosphate moiety, such as peptide nucleic acid (PNA). Nucleotide substitutes include molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes also include nucleotides or nucleotide analogs that have had the phosphate moiety or sugar moieties replaced. In some embodiments, nucleotide substitutes may not contain a standard phosphorus atom. Substitutes for the phosphate can be, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

It is also understood in a nucleotide substitute that both the sugar and the phosphate moieties of the nucleotide can be replaced by, for example, an amide type linkage (aminoethylglycine) (PNA).

It is also possible to link other types of molecules (conjugates) to nucleotides or nucleotide analogs to enhance, for example, cellular uptake. Conjugates can be chemically linked to the nucleotide or nucleotide analogs. Such conjugates include, for example, lipid moieties such as a cholesterol moiety, cholic acid, a thioether such as hexyl-S-tritylthiol, a thiocholesterol, an aliphatic chain such as dodecandiol or undecyl residues, a phospholipid such as di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety, or an octadecylamine or hexylamino-carbonyl-oxycholesterol moiety.

The disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (e.g., a circular double-stranded DNA into which additional DNA segments can be ligated). In some embodiments, the vector is a viral vector, wherein additional DNA segments can be ligated into the viral genome. In some embodiments, the vector can autonomously replicate in a host cell into which it is introduced (e.g., bacterial vectors having a bacterial origin of replication and episomal mammalian vectors). In some embodiments, the vector (e.g., non-episomal mammalian vectors) can be integrated into the genome of a host cell upon introduction into the host cell and thereby are replicated along with the host genome. Moreover, particular vectors can direct the expression of genes to which they are operatively linked. Such vectors are referred to herein as “recombinant expression vectors” or “expression vectors.” Such vectors can also be targeting vectors (i.e., exogenous donor sequences).

In some embodiments, the proteins encoded by the various genetic variants disclosed herein are expressed by inserting nucleic acid molecules encoding the disclosed genetic variants into expression vectors, such that the genes are operatively linked to expression control sequences, such as transcriptional and translational control sequences. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art. In some embodiments, nucleic acid molecules comprising the disclosed genetic variants can be ligated into a vector such that transcriptional and translational control sequences within the vector serve their intended function of regulating the transcription and translation of the genetic variant. The expression vector and expression control sequences are chosen to be compatible with the expression host cell used. Nucleic acid sequences comprising the disclosed genetic variants can be inserted into separate vectors or into the same expression vector as the variant genetic information. A nucleic acid sequence comprising the disclosed genetic variants can be inserted into the expression vector by standard methods (e.g., ligation of complementary restriction sites on the nucleic acid comprising the disclosed genetic variants and vector, or blunt end ligation if no restriction sites are present).

In addition to a nucleic acid sequence comprising the disclosed genetic variants, the recombinant expression vectors can carry regulatory sequences that control the expression of the genetic variant in a host cell. The design of the expression vector, including the selection of regulatory sequences can depend on such factors as the choice of the host cell to be transformed, the level of expression of protein desired, and so forth. Desired regulatory sequences for mammalian host cell expression can include, for example, viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. Methods of expressing polypeptides in bacterial cells or fungal cells (e.g., yeast cells) are also well known.

A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

In addition to a nucleic acid sequence comprising the disclosed genetic variants and regulatory sequences, the recombinant expression vectors can carry additional sequences, such as sequences that regulate replication of the vector in host cells (e.g., origins of replication) and selectable marker genes. A selectable marker gene can facilitate selection of host cells into which the vector has been introduced (see e.g., U.S. Pat. Nos. 4,399,216; 4,634,665; and 5,179,017). For example, a selectable marker gene can confer resistance to drugs, such as G418, hygromycin, or methotrexate, on a host cell into which the vector has been introduced. Exemplary selectable marker genes include, but are not limited to, the dihydrofolate reductase (DHFR) gene (for use in dhfr-host cells with methotrexate selection/amplification), the neo gene (for G418 selection), and the glutamate synthetase (GS) gene.

Additional vectors are described in, for example, U.S. Provisional Application No. 62/367,973, filed on Jul. 28, 2016, which is incorporated herein by reference in its entirety.

The disclosure also provides compositions comprising any one or more of the isolated nucleic acid molecules, genomic DNA molecules, cDNA molecules, or mRNA molecules disclosed herein. In some embodiments, the composition is a pharmaceutical composition.

The disclosure also provides variant SLC14A1 polypeptides. In some embodiments, the variant SLC14A1 polypeptides are loss of function polypeptides or partial loss of function polypeptides. In some embodiments, the variant SLC14A1 polypeptide comprises an isoleucine at a position corresponding to position 76 according to SEQ ID NO:13 or comprises an isoleucine at a position corresponding to position 132 according to SEQ ID NO:14. In some embodiments, the variant SLC14A1 polypeptide comprises an isoleucine at a position corresponding to position 76 according to SEQ ID NO:13. In some embodiments, the variant SLC14A1 polypeptide comprises an isoleucine at a position corresponding to position 132 according to SEQ ID NO:14. In some embodiments, the variant SLC14A1 polypeptide does not comprise or consist of SEQ ID NO:13 or SEQ ID NO:14.

In some embodiments, the variant SLC14A1 polypeptide has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:13 and comprises an isoleucine at a position corresponding to position 76 according to SEQ ID NO:13. In some embodiments, the variant SLC14A1 polypeptide comprises or consists of the amino acid sequence according to SEQ ID NO:13. In some embodiments, the variant SLC14A1 polypeptide has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:13 and comprises an isoleucine at a position corresponding to position 76 according to SEQ ID NO:13, provided that the variant SLC14A1 polypeptide does not comprise or consist of an amino acid sequence according to SEQ ID NO:13.

In some embodiments, the variant SLC14A1 polypeptide has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:14 and comprises an isoleucine at a position corresponding to position 132 according to SEQ ID NO:14. In some embodiments, the variant SLC14A1 polypeptide comprises or consists of the amino acid sequence according to SEQ ID NO:14. In some embodiments, the variant SLC14A1 polypeptide has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:14 and comprises an isoleucine at a position corresponding to position 132 according to SEQ ID NO:14, provided that the variant SLC14A1 polypeptide does not comprise or consist of an amino acid sequence according to SEQ ID NO:14.

The disclosure also provides fragments of any of the polypeptides disclosed herein. In some embodiments, the fragments comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 150, at least about 200, at least about 250, at least about 300, or at least about 350 contiguous amino acid residues of the encoded polypeptide (such as the polypeptides having the amino acid sequence of SEQ ID NO:13 and/or SEQ ID NO:14). In this regard, the longer fragments are preferred over the shorter ones. In some embodiments, the fragments comprise at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 contiguous amino acid residues of the encoded polypeptide. In this regard, the longer fragments are preferred over the shorter ones.

The disclosure also provides dimers comprising an isolated polypeptide comprising a variant SLC14A1 polypeptide wherein the polypeptide is selected from any of the polypeptides disclosed herein.

In some embodiments, the isolated polypeptides disclosed herein are linked or fused to heterologous polypeptides or heterologous molecules or labels, numerous examples of which are disclosed elsewhere herein. For example, the proteins can be fused to a heterologous polypeptide providing increased or decreased stability. The fused domain or heterologous polypeptide can be located at the N-terminus, the C-terminus, or internally within the polypeptide. A fusion partner may, for example, assist in providing T helper epitopes (an immunological fusion partner), or may assist in expressing the protein (an expression enhancer) at higher yields than the native recombinant polypeptide. Certain fusion partners are both immunological and expression enhancing fusion partners. Other fusion partners may be selected to increase the solubility of the polypeptide or to facilitate targeting the polypeptide to desired intracellular compartments. Some fusion partners include affinity tags, which facilitate purification of the polypeptide.

In some embodiments, a fusion protein is directly fused to the heterologous molecule or is linked to the heterologous molecule via a linker, such as a peptide linker. Suitable peptide linker sequences may be chosen, for example, based on the following factors: 1) the ability to adopt a flexible extended conformation; 2) the resistance to adopt a secondary structure that could interact with functional epitopes on the first and second polypeptides; and 3) the lack of hydrophobic or charged residues that might react with the polypeptide functional epitopes. For example, peptide linker sequences may contain Gly, Asn and Ser residues. Other near neutral amino acids, such as Thr and Ala may also be used in the linker sequence. Amino acid sequences which may be usefully employed as linkers include those disclosed in, for example, Maratea et al., Gene, 1985, 40, 39-46; Murphy et al., Proc. Natl. Acad. Sci. USA, 1986, 83, 8258-8262; and U.S. Pat. Nos. 4,935,233 and 4,751,180. A linker sequence may generally be, for example, from 1 to about 50 amino acids in length. Linker sequences are generally not required when the first and second polypeptides have non-essential N-terminal amino acid regions that can be used to separate the functional domains and prevent steric interference.

In some embodiments, the polypeptides are operably linked to a cell-penetrating domain. For example, the cell-penetrating domain can be derived from the HIV-1 TAT protein, the TLM cell-penetrating motif from human hepatitis B virus, MPG, Pep-1, VP22, a cell-penetrating peptide from Herpes simplex virus, or a polyarginine peptide sequence. See, e.g., WO 2014/089290. The cell-penetrating domain can be located at the N-terminus, the C-terminus, or anywhere within the protein.

In some embodiments, the polypeptides are operably linked to a heterologous polypeptide for ease of tracking or purification, such as a fluorescent protein, a purification tag, or an epitope tag. Examples of fluorescent proteins include, but are not limited to, green fluorescent proteins (e.g., GFP, GFP-2, tagGFP, turboGFP, eGFP, Emerald, Azami Green, Monomeric Azami Green, CopGFP, AceGFP, ZsGreenI), yellow fluorescent proteins (e.g., YFP, eYFP, Citrine, Venus, YPet, PhiYFP, ZsYellowI), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin. In some embodiments, the heterologous molecule is an immunoglobulin Fc domain, a peptide purification tag, a transduction domain, poly(ethylene glycol), polysialic acid, or glycolic acid.

In some embodiments, isolated polypeptides comprise non-natural or modified amino acids or peptide analogs. For example, there are numerous D-amino acids or amino acids which have a different functional substituent than the naturally occurring amino acids. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way.

In some embodiments, the isolated polypeptides are peptide mimetics, which can be produced to resemble peptides, but which are not connected via a natural peptide linkage. For example, linkages for amino acids or amino acid analogs include, but are not limited to, —CH₂NH—, —CH₂S—, —CH₂—, —CH═CH— (cis and trans), —COCH₂—, —CH(OH)CH₂—, and —CHH₂SO—. Peptide analogs can have more than one atom between the bond atoms, such as b-alanine, gaminobutyric acid, and the like. Amino acid analogs and peptide analogs often have enhanced or desirable properties, such as, more economical production, greater chemical stability, enhanced pharmacological properties (half-life, absorption, potency, efficacy, and so forth), altered specificity (e.g., a broad-spectrum of biological activities), reduced antigenicity, and others desirable properties.

In some embodiments, the isolated polypeptides comprise D-amino acids, which can be used to generate more stable peptides because D amino acids are not recognized by peptidases. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations (see, e.g., Rizo and Gierasch, Ann. Rev. Biochem., 1992, 61, 387).

The disclosure also provides nucleic acid molecules encoding any of the polypeptides disclosed herein. This includes all degenerate sequences related to a specific polypeptide sequence (all nucleic acids having a sequence that encodes one particular polypeptide sequence as well as all nucleic acids, including degenerate nucleic acids, encoding the disclosed variants and derivatives of the protein sequences). Thus, while each particular nucleic acid sequence may not be written out herein, each and every sequence is in fact disclosed and described herein through the disclosed polypeptide sequences.

Percent identity (or percent complementarity) between particular stretches of nucleic acid sequences within nucleic acids or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The disclosure also provides compositions comprising any one or more of the nucleic acid molecules and/or any one or more of the polypeptides disclosed herein and a carrier and/or excipient. In some embodiments, the carrier increases the stability of the nucleic acid molecule and/or polypeptide (e.g., prolonging the period under given conditions of storage (e.g., −20° C., 4° C., or ambient temperature) for which degradation products remain below a threshold, such as below 0.5% by weight of the starting nucleic acid or protein; or increasing the stability in vivo). Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

The disclosure also provides methods of producing any of the polypeptides or fragments thereof disclosed herein. Such polypeptides or fragments thereof can be produced by any suitable method. For example, polypeptides or fragments thereof can be produced from host cells comprising nucleic acid molecules (e.g., recombinant expression vectors) encoding such polypeptides or fragments thereof. Such methods can comprise culturing a host cell comprising a nucleic acid molecule (e.g., recombinant expression vector) encoding a polypeptide or fragment thereof under conditions sufficient to produce the polypeptide or fragment thereof, thereby producing the polypeptide or fragment thereof. The nucleic acid can be operably linked to a promoter active in the host cell, and the culturing can be carried out under conditions whereby the nucleic acid is expressed.

Such methods can further comprise recovering the expressed polypeptide or fragment thereof. The recovering can further comprise purifying the polypeptide or fragment thereof. Examples of suitable systems for protein expression include host cells such as, for example: bacterial cell expression systems (e.g., Escherichia coli, Lactococcus lactis), yeast cell expression systems (e.g., Saccharomyces cerevisiae, Pichia pastoris), insect cell expression systems (e.g., baculovirus-mediated protein expression), and mammalian cell expression systems.

Examples of nucleic acid molecules encoding polypeptides or fragments thereof are disclosed in more detail elsewhere herein. In some embodiments, the nucleic acid molecules are codon optimized for expression in the host cell. In some embodiments, the nucleic acid molecules are operably linked to a promoter active in the host cell. The promoter can be a heterologous promoter (e.g., a promoter than is not a naturally occurring promoter). Examples of promoters suitable for Escherichia coli include, but are not limited to, arabinose, lac, tac, and T7 promoters. Examples of promoters suitable for Lactococcus lactis include, but are not limited to, P170 and nisin promoters. Examples of promoters suitable for Saccharomyces cerevisiae include, but are not limited to, constitutive promoters such as alcohol dehydrogenase (ADHI) or enolase (ENO) promoters or inducible promoters such as PHO, CUP1, GAL1, and G10. Examples of promoters suitable for Pichia pastoris include, but are not limited to, the alcohol oxidase I (AOX I) promoter, the glyceraldehyde 3 phosphate dehydrogenase (GAP) promoter, and the glutathione dependent formaldehyde dehydrogenase (FLDI) promoter. An example of a promoter suitable for a baculovirus-mediated system is the late viral strong polyhedrin promoter.

In some embodiments, the nucleic acid molecules encode a tag in frame with the polypeptide or fragment thereof to facilitate protein purification. Examples of tags are disclosed elsewhere herein. Such tags can, for example, bind to a partner ligand (e.g., immobilized on a resin) such that the tagged protein can be isolated from all other proteins (e.g., host cell proteins). Affinity chromatography, high performance liquid chromatography (HPLC), and size exclusion chromatography (SEC) are examples of methods that can be used to improve the purity of the expressed protein.

Other methods can also be used to produce polypeptides or fragments thereof. For example, two or more peptides or polypeptides can be linked together by protein chemistry techniques. For example, peptides or polypeptides can be chemically synthesized using either Fmoc (9-fluorenylmethyloxycarbonyl) or Boc (tert-butyloxycarbonoyl) chemistry. Such peptides or polypeptides can be synthesized by standard chemical reactions. For example, a peptide or polypeptide can be synthesized and not cleaved from its synthesis resin, whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively. Alternately, the peptide or polypeptide can be independently synthesized in vivo as described herein. Once isolated, these independent peptides or polypeptides may be linked to form a peptide or fragment thereof via similar peptide condensation reactions.

In some embodiments, enzymatic ligation of cloned or synthetic peptide segments allow relatively short peptide fragments to be joined to produce larger peptide fragments, polypeptides, or whole protein domains (Abrahmsen et al., Biochemistry, 1991, 30, 4151). Alternately, native chemical ligation of synthetic peptides can be utilized to synthetically construct large peptides or polypeptides from shorter peptide fragments. This method can consist of a two-step chemical reaction (Dawson et al., Science, 1994, 266, 776-779). The first step can be the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate can undergo spontaneous, rapid intramolecular reaction to form a native peptide bond at the ligation site.

In some embodiments, unprotected peptide segments can be chemically linked where the bond formed between the peptide segments as a result of the chemical ligation is an unnatural (non-peptide) bond (Schnolzer et al., Science, 1992, 256, 221).

In some embodiments, the polypeptides can possess post-expression modifications such as, for example, glycosylations, acetylations, and phosphorylations, as well as other modifications known in the art, both naturally occurring and non-naturally occurring. A polypeptide may be an entire protein, or a subsequence thereof.

The disclosure also provides methods of producing any of the polypeptides disclosed herein, comprising culturing a host cell comprising a recombinant expression vectors comprising nucleic acid molecules comprising a polynucleotide capable of encoding one or more of the polypeptides disclosed herein, or its complement, thereby producing the polypeptide.

The disclosure also provides cells (e.g., recombinant host cells) comprising any one or more of the nucleic acid molecules, including vectors comprising the nucleic acid molecules, and/or any one or more of the polypeptides disclosed herein. The cells can be in vitro, ex vivo, or in vivo. Nucleic acid molecules can be linked to a promoter and other regulatory sequences so they are expressed to produce an encoded protein. Cell lines of such cells are further provided.

In some embodiments, the cell is a totipotent cell or a pluripotent cell (e.g., an embryonic stem (ES) cell such as a rodent ES cell, a mouse ES cell, or a rat ES cell). Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm). In accordance with the disclosure, the embryonic stem cells may be non-human embryonic stem cells.

In some embodiments, the cell is a primary somatic cell, or a cell that is not a primary somatic cell. Somatic cells can include any cell that is not a gamete, germ cell, gametocyte, or undifferentiated stem cell. In some embodiments, the cell can also be a primary cell. Primary cells include cells or cultures of cells that have been isolated directly from an organism, organ, or tissue. Primary cells include cells that are neither transformed nor immortal. Primary cells include any cell obtained from an organism, organ, or tissue which was not previously passed in tissue culture or has been previously passed in tissue culture but is incapable of being indefinitely passed in tissue culture. Such cells can be isolated by conventional techniques and include, for example, somatic cells, hematopoietic cells, endothelial cells, epithelial cells, fibroblasts, mesenchymal cells, keratinocytes, melanocytes, monocytes, mononuclear cells, adipocytes, preadipocytes, neurons, glial cells, hepatocytes, skeletal myoblasts, and smooth muscle cells. For example, primary cells can be derived from connective tissues, muscle tissues, nervous system tissues, or epithelial tissues.

In some embodiments, the cells may normally not proliferate indefinitely but, due to mutation or alteration, have evaded normal cellular senescence and instead can keep undergoing division. Such mutations or alterations can occur naturally or be intentionally induced. Examples of immortalized cells include, but are not limited to, Chinese hamster ovary (CHO) cells, human embryonic kidney cells (e.g., HEK 293 cells), and mouse embryonic fibroblast cells (e.g., 3T3 cells). Numerous types of immortalized cells are well known. Immortalized or primary cells include cells that are typically used for culturing or for expressing recombinant genes or proteins. In some embodiments, the cell is a differentiated cell, such as a liver cell (e.g., a human liver cell).

The cell can be from any source. For example, the cell can be a eukaryotic cell, an animal cell, a plant cell, or a fungal (e.g., yeast) cell. Such cells can be fish cells or bird cells, or such cells can be mammalian cells, such as human cells, non-human mammalian cells, rodent cells, mouse cells or rat cells. Mammals include, but are not limited to, humans, non-human primates, monkeys, apes, cats dogs, horses, bulls, deer, bison, sheep, rodents (e.g., mice, rats, hamsters, guinea pigs), livestock (e.g., bovine species such as cows, steer, etc.; ovine species such as sheep, goats, etc.; and porcine species such as pigs and boars). Birds include, but are not limited to, chickens, turkeys, ostrich, geese, ducks, etc. Domesticated animals and agricultural animals are also included. The term “non-human animal” excludes humans.

Additional host cells are described in, for example, U.S. Provisional Application No. 62/367,973, filed on Jul. 28, 2016, which is incorporated herein by reference in its entirety.

The nucleic acid molecules and polypeptides disclosed herein can be introduced into a cell by any means. Transfection protocols as well as protocols for introducing nucleic acids or proteins into cells may vary. Non-limiting transfection methods include chemical-based transfection methods using liposomes, nanoparticles, calcium, dendrimers, and cationic polymers such as DEAE-dextran or polyethylenimine. Non-chemical methods include electroporation, sono-poration, and optical transfection. Particle-based transfection includes the use of a gene gun, or magnet-assisted transfection. Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In some embodiments, nucleofection is performed using the LONZA® NUCLEOFECTOR™ system.

Introduction of nucleic acids or proteins into a cell can also be accomplished by microinjection. Microinjection of an mRNA is usually into the cytoplasm (e.g., to deliver mRNA directly to the translation machinery), while microinjection of a protein or a DNA is usually into the nucleus. Alternately, microinjection can be carried out by injection into both the nucleus and the cytoplasm: a needle can first be introduced into the nucleus and a first amount can be injected, and while removing the needle from the cell a second amount can be injected into the cytoplasm. If a nuclease agent protein is injected into the cytoplasm, the protein may comprise a nuclear localization signal to ensure delivery to the nucleus/pronucleus.

Other methods for introducing nucleic acid or proteins into a cell can include, for example, vector delivery, particle-mediated delivery, exosome-mediated delivery, lipid-nanoparticle-mediated delivery, cell-penetrating-peptide-mediated delivery, or implantable-device-mediated delivery. Methods of administering nucleic acids or proteins to a subject to modify cells in vivo are disclosed elsewhere herein. Introduction of nucleic acids and proteins into cells can also be accomplished by hydrodynamic delivery (HDD).

Other methods for introducing nucleic acid or proteins into a cell can include, for example, vector delivery, particle-mediated delivery, exosome-mediated delivery, lipid-nanoparticle-mediated delivery, cell-penetrating-peptide-mediated delivery, or implantable-device-mediated delivery. In some embodiments, a nucleic acid or protein can be introduced into a cell in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule.

The disclosure also provides probes and primers. Examples of probes and primers are disclosed above for example. The disclosure provides probes and primers comprising a nucleic acid sequence that specifically hybridizes to any of the nucleic acid molecules disclosed herein. For example, the probe or primer may comprise a nucleic acid sequence which hybridizes to any of the nucleic acid molecules described herein that encode a variant SLC14A1 protein that comprises an isoleucine at a position corresponding to position 76 according to SEQ ID NO:13 or that comprises an isoleucine at a position corresponding to position 132 according to SEQ ID NO:14, or which hybridizes to the complement of the nucleic acid molecule. In some embodiments, the probe or primer comprises a nucleic acid sequence which hybridizes to a nucleic acid molecule encoding a variant SLC14A1 protein according to SEQ ID NO:13 or SEQ ID NO:14, or which hybridizes to the complement of these nucleic acid molecules. In some embodiments, the probe or primer may comprise a nucleic acid sequence which hybridizes to any of the nucleic acid molecules described herein that encode a variant SLC14A1 protein that comprises an isoleucine at a position corresponding to position 76 according to SEQ ID NO:13, or which hybridizes to the complement of the nucleic acid molecule. In some embodiments, the probe or primer comprises a nucleic acid sequence which hybridizes to a nucleic acid molecule encoding a variant SLC14A1 protein according to SEQ ID NO:13, or which hybridizes to the complement of these nucleic acid molecules. In some embodiments, the probe or primer may comprise a nucleic acid sequence which hybridizes to any of the nucleic acid molecules described herein that encode a variant SLC14A1 protein that comprises an isoleucine at a position corresponding to position 132 according to SEQ ID NO:14, or which hybridizes to the complement of the nucleic acid molecule. In some embodiments, the probe or primer comprises a nucleic acid sequence which hybridizes to a nucleic acid molecule encoding a variant SLC14A1 protein according to SEQ ID NO:14, or which hybridizes to the complement of these nucleic acid molecules.

In some embodiments, the probe or primer comprises a nucleic acid sequence which hybridizes to a nucleic acid molecule encoding a variant SLC14A1 polypeptide that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:13 and comprises an isoleucine at a position corresponding to position 76 according to SEQ ID NO:13, or which hybridizes to the complement of this nucleic acid molecule. In some embodiments, the probe or primer comprises a nucleic acid sequence which hybridizes to a nucleic acid molecule encoding a variant SLC14A1 polypeptide that comprises or consists of the amino acid sequence according to SEQ ID NO:13, or which hybridizes to the complement of this nucleic acid molecule.

In some embodiments, the probe or primer comprises a nucleic acid sequence which hybridizes to a nucleic acid molecule encoding a variant SLC14A1 polypeptide that has at least about 90%, at least about 91%, at least about 92%, at least about 93%, at least about 94%, at least about 95%, at least about 96%, at least about 97%, at least about 98%, or at least about 99% sequence identity to the amino acid sequence according to SEQ ID NO:14 and comprises an isoleucine at a position corresponding to position 132 according to SEQ ID NO:14, or which hybridizes to the complement of this nucleic acid molecule. In some embodiments, the probe or primer comprises a nucleic acid sequence which hybridizes to a nucleic acid molecule encoding a variant SLC14A1 polypeptide that comprises or consists of the amino acid sequence according to SEQ ID NO:14, or which hybridizes to the complement of this nucleic acid molecule.

The probe or primer may comprise any suitable length, non-limiting examples of which include at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides in length. In preferred embodiments, the probe or primer comprises at least about 18 nucleotides in length. The probe or primer may comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides in length. In preferred embodiments, the probe or primer is from about 18 to about 30 nucleotides in length.

The disclosure also provides alteration-specific probes and alteration-specific primers. In some embodiments, the alteration-specific probe or alteration-specific primer comprises a nucleic acid sequence which is complementary to and/or hybridizes, or specifically hybridizes, to a nucleic acid sequence encoding a variant SLC14A1 protein that comprises an isoleucine at a position corresponding to position 76 according to SEQ ID NO:13, or to the complement thereof. In some embodiments, the alteration-specific probe or alteration-specific primer comprises a nucleic acid sequence which is complementary to and/or hybridizes, or specifically hybridizes, to a nucleic acid sequence encoding a variant SLC14A1 protein that comprises an isoleucine at a position corresponding to position 132 according to SEQ ID NO:14, or to the complement thereof.

In the context of the disclosure “specifically hybridizes” means that the probe or primer (e.g., the alteration-specific probe or alteration-specific primer) does not hybridize to a nucleic acid molecule encoding a wild type SLC14A1 protein. In some embodiments, the alteration-specific probe specifically hybridizes to the nucleic acid codon which encodes the isoleucine at a position corresponding to position 76 according to SEQ ID NO:13, or the complement thereof. In some embodiments, the alteration-specific primer, or primer pair, specifically hybridizes to a region(s) of the nucleic acid molecule encoding a variant SLC14A1 protein such that the codon which encodes the isoleucine at a position corresponding to position 76 according to SEQ ID NO:13 is encompassed within any transcript produced therefrom. In some embodiments, the alteration-specific probe specifically hybridizes to the nucleic acid codon which encodes the isoleucine at a position corresponding to position 132 according to SEQ ID NO:14, or the complement thereof. In some embodiments, the alteration-specific primer, or primer pair, specifically hybridizes to a region(s) of the nucleic acid molecule encoding a variant SLC14A1 protein such that the codon which encodes the isoleucine at a position corresponding to position 132 according to SEQ ID NO:14 is encompassed within any transcript produced therefrom.

In some embodiments, the alteration-specific probe or alteration-specific primer comprises a nucleic acid sequence which is complementary to and/or hybridizes, or specifically hybridizes, to a nucleic acid sequence encoding a variant SLC14A1 protein, wherein the protein comprises an isoleucine at a position corresponding to position 76 according to SEQ ID NO:13, or the complement thereof. In some embodiments, the alteration-specific probe or alteration-specific primer comprises a nucleic acid sequence which is complementary to and/or hybridizes, or specifically hybridizes, to a nucleic acid sequence encoding a variant SLC14A1 protein, wherein the protein comprises an isoleucine at a position corresponding to position 132 according to SEQ ID NO:14, or the complement thereof.

In some embodiments, the alteration-specific probe or alteration-specific primer comprises a nucleic acid sequence which is complementary to and/or hybridizes, or specifically hybridizes, to a genomic DNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprises an isoleucine at a position corresponding to position 76 according to SEQ ID NO:13. In some embodiments, the alteration-specific probe or alteration-specific primer comprises a nucleic acid sequence which is complementary to and/or hybridizes, or specifically hybridizes, to a genomic DNA molecule encoding a variant SLC14A1 protein having SEQ ID NO:13.

In some embodiments, the alteration-specific probe or alteration-specific primer comprises a nucleic acid sequence which is complementary to and/or hybridizes, or specifically hybridizes, to a genomic DNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprises an isoleucine at a position corresponding to position 132 according to SEQ ID NO:14. In some embodiments, the alteration-specific probe or alteration-specific primer comprises a nucleic acid sequence which is complementary to and/or hybridizes, or specifically hybridizes, to a genomic DNA molecule encoding a variant SLC14A1 protein having SEQ ID NO:14.

In some embodiments, the alteration-specific probe or alteration-specific primer comprises a nucleic acid sequence which is complementary to and/or hybridizes, or specifically hybridizes, to a variant SLC14A1 genomic DNA molecule that comprises or consists of a nucleic acid sequence comprising an adenine at a position corresponding to position 6963 according to SEQ ID NO:2. In some embodiments, the alteration-specific probe or alteration-specific primer comprises a nucleic acid sequence which is complementary to and/or hybridizes, or specifically hybridizes, to a variant SLC14A1 genomic DNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:2 and comprises an adenine at a position corresponding to position 6963 according to SEQ ID NO:2. In some embodiments, the alteration-specific probe or alteration-specific primer comprises a nucleic acid sequence which is complementary to and/or hybridizes, or specifically hybridizes, to a variant SLC14A1 genomic DNA molecule that comprises or consists of a nucleic acid sequence according to SEQ ID NO:2.

In some embodiments, the alteration-specific probe or alteration-specific primer comprises a nucleic acid sequence which is complementary to and/or hybridizes, or specifically hybridizes, to a variant SLC14A1 mRNA molecule encoding a variant SLC14A1 protein comprising an isoleucine at a position corresponding to position 76 according to SEQ ID NO:13. In some embodiments, the alteration-specific probe or alteration-specific primer comprises a nucleic acid sequence which is complementary to and/or hybridizes, or specifically hybridizes, to a variant SLC14A1 mRNA molecule encoding a variant SLC14A1 protein comprising an isoleucine at a position corresponding to position 132 according to SEQ ID NO:14.

In some embodiments, the alteration-specific probe or alteration-specific primer comprises a nucleic acid sequence which is complementary to and/or hybridizes, or specifically hybridizes, to a variant SLC14A1 mRNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprises an isoleucine at a position corresponding to position 76 according to SEQ ID NO:13. In some embodiments, the alteration-specific probe or alteration-specific primer comprises a nucleic acid sequence which is complementary to and/or hybridizes, or specifically hybridizes, to an mRNA molecule encoding a variant SLC14A1 protein having SEQ ID NO:13.

In some embodiments, the alteration-specific probe or alteration-specific primer comprises a nucleic acid sequence which is complementary to and/or hybridizes, or specifically hybridizes, to a variant SLC14A1 mRNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprises an isoleucine at a position corresponding to position 132 according to SEQ ID NO:14. In some embodiments, the alteration-specific probe or alteration-specific primer comprises a nucleic acid sequence which is complementary to and/or hybridizes, or specifically hybridizes, to an mRNA molecule encoding a variant SLC14A1 protein having SEQ ID NO:14.

In some embodiments, the alteration-specific probe or alteration-specific primer comprises a nucleic acid sequence which is complementary to and/or hybridizes, or specifically hybridizes, to a variant SLC14A1 mRNA molecule that comprises or consists of a nucleic acid sequence comprising an adenine at a position corresponding to position 226 according to SEQ ID NO:5. In some embodiments, the alteration-specific probe or alteration-specific primer comprises a nucleic acid sequence which is complementary to and/or hybridizes, or specifically hybridizes, to a variant SLC14A1 mRNA molecule that comprises the codon AUC at positions corresponding to positions 226 to 228 according to SEQ ID NO:5. In some embodiments, the alteration-specific probe or alteration-specific primer comprises a nucleic acid sequence which is complementary to and/or hybridizes, or specifically hybridizes, to a variant SLC14A1 mRNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:5 and comprises an adenine at a position corresponding to position 226 according to SEQ ID NO:5. In some embodiments, the alteration-specific probe or alteration-specific primer comprises a nucleic acid sequence which is complementary to and/or hybridizes, or specifically hybridizes, to a variant SLC14A1 mRNA molecule that comprises or consists of a nucleic acid sequence according to SEQ ID NO:5.

In some embodiments, the alteration-specific probe or alteration-specific primer comprises a nucleic acid sequence which is complementary to and/or hybridizes, or specifically hybridizes, to a variant SLC14A1 mRNA molecule that comprises or consists of a nucleic acid sequence comprising an adenine at a position corresponding to position 394 according to SEQ ID NO:6. In some embodiments, the alteration-specific probe or alteration-specific primer comprises a nucleic acid sequence which is complementary to and/or hybridizes, or specifically hybridizes, to a variant SLC14A1 mRNA molecule that comprises the codon AUC at positions corresponding to positions 394 to 396 according to SEQ ID NO:6. In some embodiments, the alteration-specific probe or alteration-specific primer comprises a nucleic acid sequence which is complementary to and/or hybridizes, or specifically hybridizes, to a variant SLC14A1 mRNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:6 and comprises an adenine at a position corresponding to position 394 according to SEQ ID NO:6. In some embodiments, the alteration-specific probe or alteration-specific primer comprises a nucleic acid sequence which is complementary to and/or hybridizes, or specifically hybridizes, to a variant SLC14A1 mRNA molecule that comprises or consists of a nucleic acid sequence according to SEQ ID NO:6.

In some embodiments, the alteration-specific probe or alteration-specific primer comprises a nucleic acid sequence which is complementary to and/or hybridizes, or specifically hybridizes, to a variant SLC14A1 cDNA molecule encoding a variant SLC14A1 protein comprising an isoleucine at a position corresponding to position 76 according to SEQ ID NO:13. In some embodiments, the alteration-specific probe or alteration-specific primer comprises a nucleic acid sequence which is complementary to and/or hybridizes, or specifically hybridizes, to a variant SLC14A1 cDNA molecule encoding a variant SLC14A1 protein comprising an isoleucine at a position corresponding to position 132 according to SEQ ID NO:14.

In some embodiments, the alteration-specific probe or alteration-specific primer comprises a nucleic acid sequence which is complementary to and/or hybridizes, or specifically hybridizes, to a variant SLC14A1 cDNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprises an isoleucine at a position corresponding to position 76 according to SEQ ID NO:13. In some embodiments, the alteration-specific probe or alteration-specific primer comprises a nucleic acid sequence which is complementary to and/or hybridizes, or specifically hybridizes, to an cDNA molecule encoding a variant SLC14A1 protein having SEQ ID NO:13.

In some embodiments, the alteration-specific probe or alteration-specific primer comprises a nucleic acid sequence which is complementary to and/or hybridizes, or specifically hybridizes, to a variant SLC14A1 cDNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprises an isoleucine at a position corresponding to position 132 according to SEQ ID NO:14. In some embodiments, the alteration-specific probe or alteration-specific primer comprises a nucleic acid sequence which is complementary to and/or hybridizes, or specifically hybridizes, to an cDNA molecule encoding a variant SLC14A1 protein having SEQ ID NO:14.

In some embodiments, the alteration-specific probe or alteration-specific primer comprises a nucleic acid sequence which is complementary to and/or hybridizes, or specifically hybridizes, to a variant SLC14A1 cDNA molecule that comprises or consists of a nucleic acid sequence comprising an adenine at a position corresponding to position 226 according to SEQ ID NO:9. In some embodiments, the alteration-specific probe or alteration-specific primer comprises a nucleic acid sequence which is complementary to and/or hybridizes, or specifically hybridizes, to a variant SLC14A1 cDNA molecule that comprises the codon AUC at positions corresponding to positions 226 to 228 according to SEQ ID NO:9. In some embodiments, the alteration-specific probe or alteration-specific primer comprises a nucleic acid sequence which is complementary to and/or hybridizes, or specifically hybridizes, to a variant SLC14A1 cDNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:9 and comprises an adenine at a position corresponding to position 226 according to SEQ ID NO:9. In some embodiments, the alteration-specific probe or alteration-specific primer comprises a nucleic acid sequence which is complementary to and/or hybridizes, or specifically hybridizes, to a variant SLC14A1 cDNA molecule that comprises or consists of a nucleic acid sequence according to SEQ ID NO:9.

In some embodiments, the alteration-specific probe or alteration-specific primer comprises a nucleic acid sequence which is complementary to and/or hybridizes, or specifically hybridizes, to a variant SLC14A1 cDNA molecule that comprises or consists of a nucleic acid sequence comprising an adenine at a position corresponding to position 394 according to SEQ ID NO:10. In some embodiments, the alteration-specific probe or alteration-specific primer comprises a nucleic acid sequence which is complementary to and/or hybridizes, or specifically hybridizes, to a variant SLC14A1 cDNA molecule that comprises the codon AUC at positions corresponding to positions 394 to 396 according to SEQ ID NO:10. In some embodiments, the alteration-specific probe or alteration-specific primer comprises a nucleic acid sequence which is complementary to and/or hybridizes, or specifically hybridizes, to a variant SLC14A1 cDNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:10 and comprises an adenine at a position corresponding to position 394 according to SEQ ID NO:10. In some embodiments, the alteration-specific probe or alteration-specific primer comprises a nucleic acid sequence which is complementary to and/or hybridizes, or specifically hybridizes, to a variant SLC14A1 cDNA molecule that comprises or consists of a nucleic acid sequence according to SEQ ID NO:10.

The disclosure also provides an isolated alteration-specific probe or primer comprising at least about 15 nucleotides and which hybridizes to a nucleic acid sequence encoding an SLC14A1 protein, wherein the alteration-specific probe or primer comprises a nucleic acid sequence which is complementary to the portion of the SLC14A1 encoding nucleic acid sequence which encodes an isoleucine at the position corresponding to position 76 according to SEQ ID NO:13, or to the complement thereof.

The disclosure also provides an isolated alteration-specific probe or primer comprising at least about 15 nucleotides and which hybridizes to a nucleic acid sequence encoding an SLC14A1 protein, wherein the alteration-specific probe or primer comprises a nucleic acid sequence which is complementary to the portion of the SLC14A1 encoding nucleic acid sequence which encodes an isoleucine at the position corresponding to position 132 according to SEQ ID NO:14, or to the complement thereof.

The disclosure also provides an isolated polypeptide comprising an amino acid sequence which is at least about 90%, at least about 95%, at least about 96%, at least about 97%, at least about 98%, or at least about 99% identical to an SLC14A1 variant polypeptide having the amino acid sequence of SEQ ID NO:13, provided that the polypeptide comprises an isoleucine at the position corresponding to position 76 according to SEQ ID NO:13. In some embodiments, the SLC14A1 variant polypeptide comprises the amino acid sequence of SEQ ID NO:13.

The disclosure also provides an isolated polypeptide comprising an amino acid sequence which is at least about 90%, at least about 95%, at least about 96%, at least about 97%, at least about 98%, or at least about 99% identical to an SLC14A1 variant polypeptide having the amino acid sequence of SEQ ID NO:14, provided that the polypeptide comprises an isoleucine at the position corresponding to position 132 according to SEQ ID NO:14. In some embodiments, the SLC14A1 variant polypeptide comprises the amino acid sequence of SEQ ID NO:14.

The disclosure also provides use of any of the isolated probes or primers described herein or the isolated alteration-specific probes or primers described herein for determining a human subject's susceptibility to developing a coagulation condition or coronary artery disease (CAD).

The length which is described above with regard to the probe or primer of the disclosure applies, mutatis mutandis, also for the alteration-specific probe or alteration-specific primer of the disclosure.

The disclosure also provides a pair of alteration-specific primers comprising two of the alteration-specific primers as described above.

In some embodiments, the probe or primer (e.g., the alteration-specific probe or alteration-specific primer) comprises DNA. In some embodiments, the probe or primer (e.g., alteration-specific probe or alteration-specific primer) comprises RNA. In some embodiments, the probe or primer (e.g., the alteration-specific probe or alteration-specific primer) hybridizes to a nucleic acid sequence encoding the variant SLC14A1 protein under stringent conditions, such as high stringent conditions.

In some embodiments, the probe comprises a label. In some embodiments, the label is a fluorescent label, a radiolabel, or biotin. In some embodiments, the length of the probe is described above. Alternately, in some embodiments, the probe comprises or consists of at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 nucleotides. The probe (e.g., the allele-specific probe) may be used, for example, to detect any of the nucleic acid molecules disclosed herein. In preferred embodiments, the probe comprises at least about 18 nucleotides in length. The probe may comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides in length. In preferred embodiments, the probe is from about 18 to about 30 nucleotides in length.

The disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern.

Solid-state substrates for use in solid supports can include any solid material to which molecules can be coupled. This includes materials such as acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A form for a solid-state substrate is a microtiter dish, such as a standard 96-well type. In some embodiments, a multiwell glass slide can be employed that normally contain one array per well. This feature allows for greater control of assay reproducibility, increased throughput and sample handling, and ease of automation. In some embodiments, the support is a microarray.

Any of the polypeptides disclosed herein can further have one or more substitutions (such as conservative amino acid substitutions), insertions, or deletions.

Insertions include, for example, amino or carboxyl terminal fusions as well as intrasequence insertions of single or multiple amino acid residues. Techniques for making substitutions at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions can be made in adjacent pairs, i.e. a deletion of 2 residues or insertion of 2 residues. Substitutions, deletions, insertions or any combination thereof may be combined to arrive at a final construct. In some embodiments, the mutations do not place the sequence out of reading frame and do not create complementary regions that could produce secondary mRNA structure.

The disclosure also provides kits for making the compositions and utilizing the methods described herein. The kits described herein can comprise an assay or assays for detecting one or more genetic variants in a sample of a subject.

In some embodiments, the kits for identification of human SLC14A1 variants utilize the compositions and methods described above. In some embodiments, a basic kit can comprise a container having at least one pair of oligonucleotide primers or probes, such as alteration-specific probes or alteration-specific primers, for a locus in any of the nucleic acid molecules disclosed herein (such as, for example, SEQ ID NO:2, SEQ ID NO:5, SEQ ID NO:6, SEQ ID NO:9, and/or SEQ ID NO:10). A kit can also optionally comprise instructions for use. A kit can also comprise other optional kit components, such as, for example, one or more of an allelic ladder directed to each of the loci amplified, a sufficient quantity of enzyme for amplification, amplification buffer to facilitate the amplification, divalent cation solution to facilitate enzyme activity, dNTPs for strand extension during amplification, loading solution for preparation of the amplified material for electrophoresis, genomic DNA as a template control, a size marker to insure that materials migrate as anticipated in the separation medium, and a protocol and manual to educate the user and limit error in use. The amounts of the various reagents in the kits also can be varied depending upon a number of factors, such as the optimum sensitivity of the process. It is within the scope of these teachings to provide test kits for use in manual applications or test kits for use with automated sample preparation, reaction set-up, detectors or analyzers.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 genomic DNA molecule encoding a variant SLC14A1 protein that comprises an isoleucine at a position corresponding to position 76 according to SEQ ID NO:13 or that comprises an isoleucine at a position corresponding to position 132 according to SEQ ID NO:14, or the complement thereof. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 genomic DNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprising an isoleucine at a position corresponding to position 76 according to SEQ ID NO:13 or to SEQ ID NO:14 and comprising an isoleucine at a position corresponding to position 132 according to SEQ ID NO:14. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 genomic DNA molecule encoding a variant SLC14A1 protein having SEQ ID NO:2.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 genomic DNA molecule that comprises or consists of a nucleic acid sequence comprising an adenine at a position corresponding to position 6963 according to SEQ ID NO:2. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 genomic DNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:2 and comprising an adenine at a position corresponding to position 6963 according to SEQ ID NO:2. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 genomic DNA molecule that comprises or consists of a nucleic acid sequence according to SEQ ID NO:2.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule encoding a variant SLC14A1 protein comprising an isoleucine at a position corresponding to position 76 according to SEQ ID NO:13. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule encoding a variant SLC14A1 protein comprising an isoleucine at a position corresponding to position 132 according to SEQ ID NO:14. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprising an isoleucine at a position corresponding to position 76 according to SEQ ID NO:13. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprising an isoleucine at a position corresponding to position 132 according to SEQ ID NO:14. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule encoding a variant SLC14A1 protein having SEQ ID NO:13. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule encoding a variant SLC14A1 protein having SEQ ID NO:14.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises or consists of a nucleic acid sequence comprising an adenine at a position corresponding to position 226 according to SEQ ID NO:5. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises the codon AUC at positions corresponding to positions 226 to 228 according to SEQ ID NO:5. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:5 and comprises an adenine at a position corresponding to position 226 according to SEQ ID NO:5. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises or consists of a nucleic acid sequence according to SEQ ID NO:5.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises or consists of a nucleic acid sequence comprising an adenine at a position corresponding to position 394 according to SEQ ID NO:6. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises the codon AUC at positions corresponding to positions 394 to 396 according to SEQ ID NO:6. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:6 and comprises an adenine at a position corresponding to position 394 according to SEQ ID NO:6. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 mRNA molecule that comprises or consists of a nucleic acid sequence according to SEQ ID NO:6.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule encoding a variant SLC14A1 protein comprising an isoleucine at a position corresponding to position 76 according to SEQ ID NO:13. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule encoding a variant SLC14A1 protein comprising an isoleucine at a position corresponding to position 132 according to SEQ ID NO:14. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:13 and comprising an isoleucine at a position corresponding to position 76 according to SEQ ID NO:13. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule encoding a variant SLC14A1 protein having at least about 90%, at least about 91%, at least about 92%, at least about 93%, at least about 94%, at least about 95%, at least about 96%, at least about 97%, at least about 98%, or at least about 99% sequence identity to SEQ ID NO:14 and comprising an isoleucine at a position corresponding to position 132 according to SEQ ID NO:14. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule encoding a variant SLC14A1 protein having SEQ ID NO:13. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule encoding a variant SLC14A1 protein having SEQ ID NO:14.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises or consists of a nucleic acid sequence comprising an adenine at a position corresponding to position 226 according to SEQ ID NO:9. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises the codon AUC at positions corresponding to positions 226 to 228 according to SEQ ID NO:9. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:9 and comprises an adenine at a position corresponding to position 226 according to SEQ ID NO:9. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises or consists of a nucleic acid sequence according to SEQ ID NO:9.

In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises or consists of a nucleic acid sequence comprising an adenine at a position corresponding to position 394 according to SEQ ID NO:10. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises the codon AUC at positions corresponding to positions 394 to 396 according to SEQ ID NO:10. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises or consists of a nucleic acid sequence that has at least about 90%, at least about 91%, at least about 92%, at least about 93%, at least about 94%, at least about 95%, at least about 96%, at least about 97%, at least about 98%, or at least about 99% sequence identity to SEQ ID NO:10 and comprises an adenine at a position corresponding to position 394 according to SEQ ID NO:10. In some embodiments, the kits comprise at least one pair of oligonucleotide primers (e.g., alteration-specific primers) for amplification, or at least one labeled oligonucleotide probe (e.g., alteration-specific probe) for detection, of a variant SLC14A1 cDNA molecule that comprises or consists of a nucleic acid sequence according to SEQ ID NO:10.

In some embodiments, any of the kits disclosed herein may further comprise any one or more of: a nucleotide ladder, protocol, an enzyme (such as an enzyme used for amplification, such as polymerase chain reaction (PCR)), dNTPs, a buffer, a salt or salts, and a control nucleic acid sample. In some embodiments, any of the kits disclosed herein may further comprise any one or more of: a detectable label, products and reagents required to carry out an annealing reaction, and instructions.

In some embodiments, the kits disclosed herein can comprise a primer or probe or an alteration-specific primer or an alteration-specific probe comprising a 3′ terminal nucleotide that hybridizes directly to an adenine at a position corresponding to position 6963 of SEQ ID NO:2, at a position corresponding to position 226 of SEQ ID NO:5 and/or SEQ ID NO:9, or at a position corresponding to position 394 of SEQ ID NO:6 and/or SEQ ID NO:10.

Those in the art understand that the detection techniques employed are generally not limiting. Rather, a wide variety of detection means are within the scope of the disclosed methods and kits, provided that they allow the presence or absence of an amplicon to be determined.

In some aspects, a kit can comprise one or more of the primers or probes disclosed herein. For example, a kit can comprise one or more probes that hybridize to one or more of the disclosed genetic variants.

In some aspects, a kit can comprise one of the disclosed cells or cell lines. In some aspects, a kit can comprise the materials necessary to create a transgenic cell or cell line. For example, in some aspects a kit can comprise a cell and a vector comprising a nucleic acid sequence comprising one or more of the disclosed genetic variants. A kit can further comprise media for cell culture.

The disclosure also provides methods for detecting the presence of an SLC14A1 variant genomic DNA, mRNA, cDNA, and/or polypeptide in a biological sample from a subject human. In some embodiments, the SLC14A1 variant genomic DNA, mRNA, and/or cDNA result in variant SLC14A1 polypeptides that have loss of function or partial loss of function. It is understood that gene sequences within a population and mRNAs and proteins encoded by such genes can vary due to polymorphisms such as single-nucleotide polymorphisms. The sequences provided herein for the SLC14A1 genomic DNA, mRNA, cDNA, and polypeptide are only exemplary sequences. Other sequences for the SLC14A1 genomic DNA, mRNA, cDNA, and polypeptide are also possible.

The disclosure also provides methods of determining whether a human subject carries an SLC14A1 variant nucleic acid molecule, comprising assaying a sample obtained from the subject to determine whether a nucleic acid molecule in the sample comprises a nucleic acid sequence that encodes an SLC14A1 protein comprising an isoleucine at the position corresponding to position 76 according to SEQ ID NO:13 and/or whether a nucleic acid molecule in the sample comprises a nucleic acid sequence that encodes an SLC14A1 protein comprising an isoleucine at the position corresponding to position 132 according to SEQ ID NO:14. In some embodiments, if in the sample a nucleic acid molecule is identified which comprises a nucleic acid sequence that encodes an SLC14A1 protein comprising an isoleucine at the position corresponding to position 76 according to SEQ ID NO:13 and/or if in the sample a nucleic acid molecule is identified which comprises a nucleic acid sequence that encodes an SLC14A1 protein comprising an isoleucine at the position corresponding to position 132 according to SEQ ID NO:14, then the human subject is classified as being at decreased risk for developing a coagulation condition or coronary artery disease (CAD). In some embodiments, if in the sample a nucleic acid molecule is identified which comprises a nucleic acid sequence that encodes an SLC14A1 protein which does not comprise an isoleucine at the position corresponding to position 76 according to SEQ ID NO:13 and/or if in the sample a nucleic acid molecule is identified which comprises a nucleic acid sequence that encodes an SLC14A1 protein which does not comprise an isoleucine at the position corresponding to position 132 according to SEQ ID NO:14, then the human subject is classified as being at increased risk for developing a coagulation condition or CAD. In some embodiments, the coagulation condition is chosen from thrombosis, pulmonary embolism, myocardial infarction (MI), venous thromboembolism (VTE), deep vein thrombosis (DVT), cerebral aneurysm, and stroke.

The disclosure also provides methods of determining whether a human subject carries an SLC14A1 Va1761Ile protein and/or an SLC14A1 Va1132Ile protein, comprising performing an assay on a sample obtained from the human subject to determine whether an SLC14A1 protein in the sample comprises an isoleucine at the position corresponding to position 76 according to SEQ ID NO:13 and/or whether an SLC14A1 protein in the sample comprises an isoleucine at the position corresponding to position 132 according to SEQ ID NO:14. In some embodiments, if in the sample an SLC14A1 protein is identified which comprises an isoleucine at the position corresponding to position 76 according to SEQ ID NO:13 and/or if in the sample an SLC14A1 protein is identified which comprises an isoleucine at the position corresponding to position 132 according to SEQ ID NO:14, then the human subject is classified as being at decreased risk for developing a coagulation condition or coronary artery disease (CAD). In some embodiments, if in the sample an SLC14A1 protein is identified which does not comprise an isoleucine at the position corresponding to position 76 according to SEQ ID NO:13 and/or if in the sample an SLC14A1 protein is identified which does not comprise an isoleucine at the position corresponding to position 132 according to SEQ ID NO:14, then the human subject is classified as being at increased risk for developing a coagulation condition or CAD. In some embodiments, the coagulation condition is chosen from thrombosis, pulmonary embolism, myocardial infarction (MI), venous thromboembolism (VTE), deep vein thrombosis (DVT), cerebral aneurysm, and stroke. In some embodiments, an enzyme-linked immunosorbent assay (ELISA) is used for determining whether an SLC14A1 protein in the sample comprises an isoleucine at the position corresponding to position 76 according to SEQ ID NO:13 and/or whether an SLC14A1 protein in the sample comprises an isoleucine at the position corresponding to position 132 according to SEQ ID NO:14. In some embodiments, the method is an in vitro method.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting a variant SLC14A1 nucleic acid molecule, preliminary processing designed to isolate or enrich the sample for the genomic DNA can be employed. A variety of known techniques may be used for this purpose. When detecting the level of variant SLC14A1 mRNA, different techniques can be used enrich the biological sample with mRNA. Various methods to detect the presence or level of a mRNA or the presence of a particular variant genomic DNA locus can be used.

The disclosure also provides methods of detecting an SLC14A1 variant nucleic acid molecule in a human subject, wherein the SLC14A1 variant nucleic acid molecule encodes a loss of function SLC14A1 protein or a partial loss of function SLC14A1 protein. In some embodiments, the method of detecting an SLC14A1 variant nucleic acid molecule in a human subject comprises assaying a sample obtained from the subject to determine whether a nucleic acid molecule in the sample comprises a nucleic acid sequence that encodes an isoleucine at the position corresponding to position 76 according to SEQ ID NO:13 or whether a nucleic acid molecule in the sample comprises a nucleic acid sequence that encodes an isoleucine at the position corresponding to position 132 according to SEQ ID NO:14.

The disclosure also provides methods of detecting the presence or absence of a variant SLC14A1 protein in a human subject, wherein the SLC14A1 variant protein is a loss of function SLC14A1 protein or a partial loss of function SLC14A1 protein. In some embodiments, the method of detecting the presence or absence of a variant SLC14A1 protein comprises sequencing at least a portion of a protein in a biological sample to determine whether the protein comprises an amino acid sequence encoding an SLC14A1 protein comprising an isoleucine at the position corresponding to position 76 according to SEQ ID NO:13 or comprising an isoleucine at the position corresponding to position 132 according to SEQ ID NO:14.

In some embodiments, the disclosure provides methods of detecting the presence or absence of a variant SLC14A1 nucleic acid molecule comprising sequencing at least a portion of a nucleic acid in a biological sample to determine whether the nucleic acid comprises a nucleic acid sequence encoding an SLC14A1 protein comprising an isoleucine at the position corresponding to position 76 according to SEQ ID NO:13 or comprising an isoleucine at the position corresponding to position 132 according to SEQ ID NO:14. Any of the variant nucleic acid molecules disclosed herein can be detected using any of the probes and primers described herein.

In some embodiments, the methods of detecting the presence or absence of a coagulation condition-associated variant SLC14A1 nucleic acid molecule or CAD-associated variant SLC14A1 nucleic acid molecule (e.g., genomic DNA, mRNA, or cDNA) in a subject, comprises: performing an assay on a biological sample obtained from the subject, which assay determines whether a nucleic acid molecule in the biological sample comprises a variant SLC14A1 nucleic acid molecule encoding a loss of function SLC14A1 protein or partial loss of function SLC14A1 protein.

In some embodiments, the methods of detecting the presence or absence of a coagulation condition-associated variant SLC14A1 nucleic acid molecule or CAD-associated variant SLC14A1 nucleic acid molecule (e.g., genomic DNA, mRNA, or cDNA) in a subject, comprises: performing an assay on a biological sample obtained from the subject, which assay determines whether a nucleic acid molecule in the biological sample comprises any of the variant SLC14A1 nucleic acid sequences disclosed herein (e.g., a nucleic acid molecule that encodes an SLC14A1 protein comprising an isoleucine at the position corresponding to position 76 according to SEQ ID NO:13 or comprising an isoleucine at the position corresponding to position 132 according to SEQ ID NO:14). In some embodiments, the biological sample comprises a cell or cell lysate. Such methods can further comprise, for example, obtaining a biological sample from the subject comprising an SLC14A1 genomic DNA or mRNA, and if mRNA, optionally reverse transcribing the mRNA into cDNA, and performing an assay on the biological sample that determine whether a position of the SLC14A1 genomic DNA, mRNA, or cDNA encodes an SLC14A1 protein comprising an isoleucine at the position corresponding to position 76 according to SEQ ID NO:13 or comprising an isoleucine at the position corresponding to position 132 according to SEQ ID NO:14. Such assays can comprise, for example determining the identity of these positions of the particular SLC14A1 nucleic acid molecule. In some embodiments, the subject is a human.

In some embodiments, the assay comprises: sequencing at least a portion of the SLC14A1 genomic DNA sequence of a nucleic acid molecule in the biological sample from the subject, wherein the portion sequenced includes the position corresponding to the position encoding an isoleucine at a position corresponding to position 76 according to SEQ ID NO:13 or wherein the portion sequenced includes the position corresponding to the position encoding an isoleucine at a position corresponding to position 132 according to SEQ ID NO:14; sequencing at least a portion of the SLC14A1 mRNA sequence of a nucleic acid molecule in the biological sample from the subject, wherein the portion sequenced includes the position corresponding to the position encoding an isoleucine at a position corresponding to position 76 according to SEQ ID NO:13 or wherein the portion sequenced includes the position corresponding to the position encoding an isoleucine at a position corresponding to position 132 according to SEQ ID NO:14; or sequencing at least a portion of the SLC14A1 cDNA sequence of a nucleic acid molecule in the biological sample from the subject, wherein the portion sequenced includes the position corresponding to the position encoding an isoleucine at a position corresponding to position 76 according to SEQ ID NO:13 or wherein the portion sequenced includes the position corresponding to the position encoding an isoleucine at a position corresponding to position 132 according to SEQ ID NO:14.

In some embodiments, the assay comprises: a) contacting the biological sample with a primer hybridizing to: i) a portion of the SLC14A1 genomic DNA sequence that is proximate to the positions of the SLC14A1 genomic sequence at the position corresponding to the position encoding an isoleucine at a position corresponding to position 76 according to SEQ ID NO:13 or a portion of the SLC14A1 genomic DNA sequence that is proximate to the positions of the SLC14A1 genomic sequence at the position corresponding to the position encoding an isoleucine at a position corresponding to position 132 according to SEQ ID NO:14; ii) a portion of the SLC14A1 mRNA sequence that is proximate to the positions of the SLC14A1 genomic sequence at the position corresponding to the position encoding an isoleucine at a position corresponding to position 76 according to SEQ ID NO:13 or a portion of the SLC14A1 mRNA sequence that is proximate to the positions of the SLC14A1 genomic sequence at the position corresponding to the position encoding an isoleucine at a position corresponding to position 132 according to SEQ ID NO:14; or iii) a portion of the SLC14A1 cDNA sequence that is proximate to the positions of the SLC14A1 genomic sequence at the position corresponding to the position encoding an isoleucine at a position corresponding to position 76 according to SEQ ID NO:13 or a portion of the SLC14A1 cDNA sequence that is proximate to the positions of the SLC14A1 genomic sequence at the position corresponding to the position encoding an isoleucine at a position corresponding to position 132 according to SEQ ID NO:14; b) extending the primer at least through: i) the positions of the SLC14A1 genomic DNA sequence corresponding to nucleotide positions beyond the codon encoding an isoleucine at a position corresponding to position 76 according to SEQ ID NO:13 or the position of the SLC14A1 genomic DNA sequence corresponding to nucleotide positions beyond the codon encoding an isoleucine at a position corresponding to position 132 according to SEQ ID NO:14; ii) the position of the SLC14A1 mRNA sequence corresponding to nucleotide positions beyond the codon encoding an isoleucine at a position corresponding to position 76 according to SEQ ID NO:13 or the position of the SLC14A1 mRNA sequence corresponding to nucleotide positions beyond the codon encoding an isoleucine at a position corresponding to position 132 according to SEQ ID NO:14; or iii) the position of the SLC14A1 cDNA sequence corresponding to nucleotide positions beyond the codon encoding an isoleucine at a position corresponding to position 76 according to SEQ ID NO:13 or the position of the SLC14A1 cDNA sequence corresponding to nucleotide positions beyond the codon encoding an isoleucine at a position corresponding to position 132 according to SEQ ID NO:14; and c) determining whether the extension product of the primer comprises nucleotides encoding an isoleucine at a position corresponding to position 76 according to SEQ ID NO:13 or determining whether the extension product of the primer comprises nucleotides encoding an isoleucine at a position corresponding to position 132 according to SEQ ID NO:14. In some embodiments, only SLC14A1 genomic DNA is analyzed. In some embodiments, only SLC14A1 mRNA is analyzed. In some embodiments, only SLC14A1 cDNA obtained from SLC14A1 mRNA is analyzed.

In some embodiments, the assay comprises: a) contacting the biological sample with an alteration-specific primer hybridizing to i) a portion of the SLC14A1 genomic DNA sequence including the nucleotides encoding an isoleucine at a position corresponding to position 76 according to SEQ ID NO:13 or a portion of the SLC14A1 genomic DNA sequence including the nucleotides encoding an isoleucine at a position corresponding to position 132 according to SEQ ID NO:14; ii) a portion of the SLC14A1 mRNA sequence including the nucleotides encoding an isoleucine at a position corresponding to position 76 according to SEQ ID NO:13 or a portion of the SLC14A1 mRNA sequence including the nucleotides encoding an isoleucine at a position corresponding to position 132 according to SEQ ID NO:14; or iii) a portion of the SLC14A1 cDNA sequence including the nucleotides encoding an isoleucine at a position corresponding to position 76 according to SEQ ID NO:13 or a portion of the SLC14A1 cDNA sequence including the nucleotides encoding an isoleucine at a position corresponding to position 132 according to SEQ ID NO:14; b) extending the primer using an alteration-specific polymerase chain reaction technique; and c) determining whether extension occurred. Alteration-specific polymerase chain reaction techniques can be used to detect mutations such as deletions in a nucleic acid sequence. Alteration-specific primers are used because the DNA polymerase will not extend when a mismatch with the template is present. A number of variations of the basic alteration-specific polymerase chain reaction technique are at the disposal of the skilled artisan.

The alteration-specific primer may comprise a nucleic acid sequence which is complementary to a nucleic acid sequence encoding the SLC14A1 protein comprising an isoleucine at a position corresponding to position 76 according to SEQ ID NO:13 or comprising an isoleucine at a position corresponding to position 132 according to SEQ ID NO:14, or the complement to the nucleic acid sequence. For example, the alteration-specific primer may comprise a nucleic acid sequence which is complementary to the nucleic acid sequence encoding SEQ ID NO:13, or to the complement to this nucleic acid sequence. Alternately, the alteration-specific primer may comprise a nucleic acid sequence which is complementary to the nucleic acid sequence encoding SEQ ID NO:14, or to the complement to this nucleic acid sequence. The alteration-specific primer preferably specifically hybridizes to the nucleic acid sequence encoding the variant SLC14A1 protein when the nucleic acid sequence encodes an isoleucine at the position corresponding to position 76 according to SEQ ID NO:13 or encodes an isoleucine at the position corresponding to position 132 according to SEQ ID NO:14.

In some embodiments, the assay comprises: sequencing a portion of the SLC14A1 genomic sequence of a nucleic acid molecule in the sample, wherein the portion sequenced includes the positions corresponding to positions 6963 to 6965 according to SEQ ID NO:2; sequencing a portion of the SLC14A1 mRNA sequence of a nucleic acid molecule in the sample, wherein the portion sequenced includes the positions corresponding to positions 226 to 228 according to SEQ ID NO:5; sequencing a portion of the SLC14A1 mRNA sequence of a nucleic acid molecule in the sample, wherein the portion sequenced includes the positions corresponding to positions 394 to 396 according to SEQ ID NO:6; sequencing a portion of the SLC14A1 cDNA sequence of a nucleic acid molecule in the sample, wherein the portion sequenced includes the positions corresponding to positions 226 to 228 according to SEQ ID NO:9; and/or sequencing a portion of the SLC14A1 cDNA sequence of a nucleic acid molecule in the sample, wherein the portion sequenced includes the positions corresponding to positions 394 to 396 according to SEQ ID NO:10.

In some embodiments, the assay comprises: a) contacting the sample with a primer hybridizing to: i) a portion of the SLC14A1 genomic sequence that is proximate to the positions of the SLC14A1 genomic sequence corresponding to positions 6963 to 6965 according to SEQ ID NO:2; ii) a portion of the SLC14A1 mRNA sequence that is proximate to the positions of the SLC14A1 mRNA corresponding to positions 226 to 228 according to SEQ ID NO:5 or corresponding to positions 394 to 396 according to SEQ ID NO:6; or iii) a portion of the SLC14A1 cDNA sequence that is proximate to the positions of the SLC14A1 cDNA corresponding to positions 226 to 228 according to SEQ ID NO:9 or corresponding to positions 394 to 396 according to SEQ ID NO:10; b) extending the primer at least through: i) the positions of the SLC14A1 genomic nucleic acid sequence corresponding to positions 6963 to 6965 according to SEQ ID NO:2; ii) the positions of the SLC14A1 mRNA nucleic acid sequence corresponding to positions 226 to 228 according to SEQ ID NO:5 or corresponding to positions 394 to 396 according to SEQ ID NO:6; or iii) the positions of the SLC14A1 cDNA nucleic acid sequence corresponding to positions 226 to 228 according to SEQ ID NO:9 or corresponding to positions 394 to 396 according to SEQ ID NO:10; and c) determining the whether the extension product of the primer comprises a codon at the positions: i) corresponding to positions 6963 to 6965 of the SLC14A1 genomic nucleic acid sequence according to SEQ ID NO:2, that encodes an isoleucine; ii) corresponding to positions 226 to 228 of the SLC14A1 mRNA according to SEQ ID NO:5 or corresponding to positions 394 to 396 of the SLC14A1 mRNA nucleic acid sequence according to SEQ ID NO:6, that encodes an isoleucine; or iii) corresponding to positions 226 to 228 of the SLC14A1 cDNA nucleic acid sequence according to SEQ ID NO:9 or corresponding to positions 394 to 396 of the SLC14A1 cDNA nucleic acid sequence according to SEQ ID NO:10, that encodes an isoleucine; that encode an isoleucine at the position corresponding to position 76 according to SEQ ID NO:13 or that encode an isoleucine at the position corresponding to position 132 according to SEQ ID NO:14.

In some embodiments, the assay comprises contacting the biological sample with a primer or probe that specifically hybridizes to a variant SLC14A1 genomic DNA sequence, mRNA sequence, or cDNA sequence and not the corresponding wild type SLC14A1 sequence under stringent conditions, and determining whether hybridization has occurred.

In some embodiments, the assay comprises RNA sequencing (RNA-Seq). In some embodiments, the assays also comprise reverse transcribing mRNA into cDNA via the reverse transcriptase polymerase chain reaction (RT-PCR).

In some embodiments, the methods utilize probes and primers of sufficient nucleotide length to bind to the target nucleic acid sequence and specifically detect and/or identify a polynucleotide comprising a variant SLC14A1 genomic DNA, mRNA, or cDNA. The hybridization conditions or reaction conditions can be determined by the operator to achieve this result. This nucleotide length may be any length that is sufficient for use in a detection method of choice, including any assay described or exemplified herein. Generally, for example, primers or probes having about 8, about 10, about 11, about 12, about 14, about 15, about 16, about 18, about 20, about 22, about 24, about 26, about 28, about 30, about 40, about 50, about 75, about 100, about 200, about 300, about 400, about 500, about 600, or about 700 nucleotides, or more, or from about 11 to about 20, from about 20 to about 30, from about 30 to about 40, from about 40 to about 50, from about 50 to about 100, from about 100 to about 200, from about 200 to about 300, from about 300 to about 400, from about 400 to about 500, from about 500 to about 600, from about 600 to about 700, or from about 700 to about 800, or more nucleotides in length are used. In preferred embodiments, the probe or primer comprises at least about 18 nucleotides in length. The probe or primer may comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides in length. In preferred embodiments, the probe or primer is from about 18 to about 30 nucleotides in length.

Such probes and primers can hybridize specifically to a target sequence under high stringency hybridization conditions. Probes and primers may have complete nucleic acid sequence identity of contiguous nucleotides with the target sequence, although probes differing from the target nucleic acid sequence and that retain the ability to specifically detect and/or identify a target nucleic acid sequence may be designed by conventional methods. Accordingly, probes and primers can share about 80%, about 85%, about 90%, about 91%, about 92%, about 93%, about 94%, about 95%, about 96%, about 97%, about 98%, about 99%, or 100% sequence identity or complementarity to the target nucleic acid molecule.

In some embodiments, specific primers can be used to amplify the variant SLC14A1 locus and/or SLC14A1 variant mRNA or cDNA to produce an amplicon that can be used as a specific probe or can itself be detected for identifying the variant SLC14A1 locus or for determining the level of specific SLC14A1 mRNA or cDNA in a biological sample. The SLC14A1 variant locus can be used to denote a genomic nucleic acid sequence including positions corresponding to positions encoding an isoleucine at position 76 according to SEQ ID NO:13 or encoding an isoleucine at position 132 according to SEQ ID NO:14. When the probe is hybridized with a nucleic acid molecule in a biological sample under conditions that allow for the binding of the probe to the nucleic acid molecule, this binding can be detected and allow for an indication of the presence of the variant SLC14A1 locus or the presence or the level of variant SLC14A1 mRNA or cDNA in the biological sample. Such identification of a bound probe has been described. The specific probe may comprise a sequence of at least about 80%, from about 80% to about 85%, from about 85% to about 90%, from about 90% to about 95%, and from about 95% to about 100% identical (or complementary) to a specific region of a variant SLC14A1 gene. The specific probe may comprise a sequence of at least about 80%, from about 80% to about 85%, from about 85% to about 90%, from about 90% to about 95%, and from about 95% to about 100% identical (or complementary) to a specific region of a variant SLC14A1 mRNA. The specific probe may comprise a sequence of at least about 80%, from about 80% to about 85%, from about 85% to about 90%, from about 90% to about 95%, and from about 95% to about 100% identical (or complementary) to a specific region of a variant SLC14A1 cDNA.

In some embodiments, to determine whether the nucleic acid complement of a biological sample comprises a nucleic acid sequence encoding the variant SLC14A1 protein (e.g., encoding an isoleucine at the position corresponding to position 76 according to SEQ ID NO:13 or encoding an isoleucine at the position corresponding to position 132 according to SEQ ID NO:14), the biological sample may be subjected to a nucleic acid amplification method using a primer pair that includes a first primer derived from the 5′ flanking sequence adjacent to positions encoding the isoleucine at the position corresponding to position 76 according to SEQ ID NO:13 or encoding the isoleucine at the position corresponding to position 132 according to SEQ ID NO:14, and a second primer derived from the 3′ flanking sequence adjacent to positions encoding the isoleucine at the position corresponding to position 76 according to SEQ ID NO:13 or encoding the isoleucine at the position corresponding to position 132 according to SEQ ID NO:14, to produce an amplicon that is diagnostic for the presence of the nucleotides at positions encoding the serine at the position corresponding to position 186 according to SEQ ID NO:9. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions encoding the isoleucine at position 76 according to SEQ ID NO:13 or encoding the isoleucine at the position corresponding to position 132 according to SEQ ID NO:14 and at least 1, 2, 3, 4, 5, 6, 7, 8, 9, 10, or more nucleotides on each side of positions encoding the isoleucine at position 76 according to SEQ ID NO:13 or encoding the isoleucine at the position corresponding to position 132 according to SEQ ID NO:14. Similar amplicons can be generated from the mRNA and/or cDNA sequences.

Representative methods for preparing and using probes and primers are described, for example, in Molecular Cloning: A Laboratory Manual, 2nd Ed., Vol. 1-3, ed. Sambrook et al., Cold Spring Harbor Laboratory Press, Cold Spring Harbor, N.Y. 1989 (hereinafter, “Sambrook et al., 1989”); Current Protocols in Molecular Biology, ed. Ausubel et al., Greene Publishing and Wiley-lnterscience, New York, 1992 (with periodic updates) (hereinafter, “Ausubel et al., 1992”); and Innis et al., PCR Protocols: A Guide to Methods and Applications, Academic Press: San Diego, 1990). PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Any nucleic acid hybridization or amplification or sequencing method can be used to specifically detect the presence of the variant SLC14A1 gene locus and/or the level of variant SLC14A1 mRNA or cDNA produced from mRNA. In some embodiments, the nucleic acid molecule can be used either as a primer to amplify a region of the SLC14A1 nucleic acid or the nucleic acid molecule can be used as a probe that specifically hybridizes, for example, under stringent conditions, to a nucleic acid molecule comprising the variant SLC14A1 gene locus or a nucleic acid molecule comprising a variant SLC14A1 mRNA or cDNA produced from mRNA.

A variety of techniques are available in the art including, for example, nucleic acid sequencing, nucleic acid hybridization, and nucleic acid amplification. Illustrative examples of nucleic acid sequencing techniques include, but are not limited to, chain terminator (Sanger) sequencing and dye terminator sequencing.

Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

Any method can be used for detecting either the non-amplified or amplified polynucleotides including, for example, Hybridization Protection Assay (HPA), quantitative evaluation of the amplification process in real-time, and determining the quantity of target sequence initially present in a sample, but which is not based on a real-time amplification.

Also provided are methods for identifying nucleic acids which do not necessarily require sequence amplification and are based on, for example, the known methods of Southern (DNA:DNA) blot hybridizations, in situ hybridization (ISH), and fluorescence in situ hybridization (FISH) of chromosomal material. Southern blotting can be used to detect specific nucleic acid sequences. In such methods, nucleic acid that is extracted from a sample is fragmented, electrophoretically separated on a matrix gel, and transferred to a membrane filter. The filter bound nucleic acid is subject to hybridization with a labeled probe complementary to the sequence of interest. Hybridized probe bound to the filter is detected. In any such methods, the process can include hybridization using any of the probes described or exemplified herein.

In hybridization techniques, stringent conditions can be employed such that a probe or primer will specifically hybridize to its target. In some embodiments, a polynucleotide primer or probe under stringent conditions will hybridize to its target sequence (e.g., the variant SLC14A1 gene locus, variant SLC14A1 mRNA, or variant SLC14A1 cDNA) to a detectably greater degree than to other sequences (e.g., the corresponding wild type SLC14A1 locus, wild type mRNA, or wild type cDNA), such as, at least 2-fold, at least 3-fold, at least 4-fold, or more over background, including over 10-fold over background. In some embodiments, a polynucleotide primer or probe under stringent conditions will hybridize to its target sequence to a detectably greater degree than to other sequences by at least 2-fold. In some embodiments, a polynucleotide primer or probe under stringent conditions will hybridize to its target sequence to a detectably greater degree than to other sequences by at least 3-fold. In some embodiments, a polynucleotide primer or probe under stringent conditions will hybridize to its target sequence to a detectably greater degree than to other sequences by at least 4-fold. In some embodiments, a polynucleotide primer or probe under stringent conditions will hybridize to its target sequence to a detectably greater degree than to other sequences by over 10-fold over background. Stringent conditions are sequence-dependent and will be different in different circumstances. By controlling the stringency of the hybridization and/or washing conditions, target sequences that are 100% complementary to the probe can be identified (homologous probing). Alternately, stringency conditions can be adjusted to allow some mismatching in sequences so that lower degrees of identity are detected (heterologous probing).

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In hybridization reactions, specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 1984, 138, 267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≥90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1° C., 2° C., 3° C., or 4° C. lower than the thermal melting point (Tₘ); moderately stringent conditions can utilize a hybridization and/or wash at 6° C., 7° C., 8° C., 9° C., or 10° C. lower than the thermal melting point (Tₘ); low stringency conditions can utilize a hybridization and/or wash at 11° C., 12° C., 13° C., 14° C., 15° C., or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used.

Also provided are methods for detecting the presence or quantifying the levels of variant SLC14A1 polypeptides in a biological sample, including, for example, protein sequencing and immunoassays. In some embodiments, the method of detecting the presence of variant SLC14A1 protein (e.g., a loss of function SLC14A1 protein or partial loss of function SLC14A1 protein) in a human subject comprises performing an assay on a biological sample from the human subject that detects the presence of the variant SLC14A1 protein (e.g., a loss of function SLC14A1 protein or partial loss of function SLC14A1 protein) in the biological sample. In some embodiments, the method of detecting the presence of variant SLC14A1 protein (e.g., SEQ D NO:13 and/or SEQ ID NO:14) in a human subject comprises performing an assay on a biological sample from the human subject that detects the presence of the variant SLC14A1 protein (e.g., SEQ D NO:13 and/or SEQ ID NO:14) in the biological sample.

Illustrative non-limiting examples of protein sequencing techniques include, but are not limited to, mass spectrometry and Edman degradation. Illustrative examples of immunoassays include, but are not limited to, immunoprecipitation, Western blot, immunohistochemistry, ELISA, immunocytochemistry, flow cytometry, and immuno-PCR. Polyclonal or monoclonal antibodies detectably labeled using various known techniques (e.g., calorimetric, fluorescent, chemiluminescent, or radioactive) are suitable for use in the immunoassays.

The disclosure also provides methods for modifying a cell, comprising introducing an expression vector into the cell, wherein the expression vector comprises a variant SLC14A1 gene comprising a nucleotide sequence encoding a loss of function SLC14A1 protein or partial loss of function SLC14A1 protein.

The disclosure also provides methods for modifying a cell, comprising introducing an expression vector into the cell, wherein the expression vector comprises a variant SLC14A1 gene comprising a nucleotide sequence encoding an isoleucine at positions corresponding to positions 6963 to 6965 according to SEQ ID NO:2. In some embodiments, the expression vector comprises a recombinant SLC14A1 gene comprising a nucleotide sequence that comprises a codon at the positions corresponding to positions 6963 to 6965 according to SEQ ID NO:2 which encodes an isoleucine. In some embodiments, the method is an in vitro method.

The disclosure also provides methods for modifying a cell, comprising introducing an expression vector into the cell, wherein the expression vector comprises a nucleic acid molecule encoding a variant SLC14A1 polypeptide that is at least about 90%, at least about 95%, at least about 96%, at least about 97%, at least about 98%, or at least about 99% identical to SEQ ID NO:13, and comprises an isoleucine at the position corresponding to position 76 according to SEQ ID NO:13. In some embodiments, the method is an in vitro method.

The disclosure also provides methods for modifying a cell, comprising introducing an expression vector into the cell, wherein the expression vector comprises a nucleic acid molecule encoding an SLC14A1 polypeptide that is at least about 90%, at least about 95%, at least about 96%, at least about 97%, at least about 98%, or at least about 99% identical to SEQ ID NO:14, and comprises an isoleucine at the position corresponding to position 132 according to SEQ ID NO:14. In some embodiments, the method is an in vitro method.

The disclosure also provides methods for modifying a cell, comprising introducing a variant SLC14A1 polypeptide, or fragment thereof, into the cell, wherein the SLC14A1 polypeptide is at least about 90%, at least about 95%, at least about 96%, at least about 97%, at least about 98%, or at least about 99% identical to SEQ ID NO:13, and comprises an isoleucine at the position corresponding to position 76 according to SEQ ID NO:13. In some embodiments, the method is an in vitro method.

The disclosure also provides methods for modifying a cell, comprising introducing a variant SLC14A1 polypeptide, or fragment thereof, into the cell, wherein the SLC14A1 polypeptide is at least about 90%, at least about 95%, at least about 96%, at least about 97%, at least about 98%, or at least about 99% identical to SEQ ID NO:14, and comprises an isoleucine at the position corresponding to position 132 according to SEQ ID NO:14. In some embodiments, the method is an in vitro method.

The disclosure also provides methods of determining a human subject's susceptibility to developing a coagulation condition or CAD. In some embodiments, the methods comprise detecting the presence of the variant SLC14A1 genomic DNA, mRNA, or cDNA obtained from mRNA, wherein the variant SLC14A1 genomic DNA, mRNA, or cDNA obtained from mRNA encodes a loss of function SLC14A1 protein or partial loss of function SLC14A1 protein.

In some embodiments, the methods comprise detecting the presence of the variant SLC14A1 genomic DNA, mRNA, or cDNA obtained from mRNA, obtained from a biological sample obtained from the subject. It is understood that gene sequences within a population and mRNAs encoded by such genes can vary due to polymorphisms such as single nucleotide polymorphisms (SNPs). The sequences provided herein for the variant SLC14A1 genomic DNA, mRNA, cDNA, and polypeptide are only exemplary sequences and other such sequences, including additional SLC14A1 alleles are also possible.

In some embodiments, the methods comprise a) assaying a sample obtained from the subject to determine whether a nucleic acid molecule in the sample comprises a nucleic acid sequence that encodes a loss of function SLC14A1 protein or partial loss of function SLC14A1 protein; and b) classifying the human subject as being at decreased risk for developing the coagulation condition or CAD if the nucleic acid molecule comprises a nucleic acid sequence that encodes a loss of function SLC14A1 protein or partial loss of function SLC14A1 protein, or classifying the human subject as being at increased risk for developing the coagulation condition or CAD if the nucleic acid molecule does not comprise a nucleic acid sequence that encodes a loss of function SLC14A1 protein or partial loss of function SLC14A1 protein.

In some embodiments, the methods comprise a) assaying a sample obtained from the subject to determine whether a nucleic acid molecule in the sample comprises a nucleic acid sequence that encodes an isoleucine at a position corresponding to position 76 according to SEQ ID NO:13 or encodes an isoleucine at a position corresponding to position 132 according to SEQ ID NO:14; and b) classifying the human subject as being at decreased risk for developing the coagulation condition or CAD if the nucleic acid molecule comprises a nucleic acid sequence that encodes an isoleucine at a position corresponding to position 76 according to SEQ ID NO:13 or encodes an isoleucine at a position corresponding to position 132 according to SEQ ID NO:14, or classifying the human subject as being at increased risk for developing the coagulation condition or CAD if the nucleic acid molecule does not comprise a nucleic acid sequence that encodes an isoleucine at a position corresponding to position 76 according to SEQ ID NO:13 or encodes an isoleucine at a position corresponding to position 132 according to SEQ ID NO:14.

In some embodiments, the assay comprises: sequencing a portion of the SLC14A1 genomic sequence of a nucleic acid molecule in the sample, wherein the portion sequenced includes the positions corresponding to positions 6963 to 6965 according to SEQ ID NO:2; sequencing a portion of the SLC14A1 mRNA sequence of a nucleic acid molecule in the sample, wherein the portion sequenced includes the positions corresponding to positions 226 to 228 according to SEQ ID NO:5; sequencing a portion of the SLC14A1 mRNA sequence of a nucleic acid molecule in the sample, wherein the portion sequenced includes the positions corresponding to positions 394 to 396 according to SEQ ID NO:6; sequencing a portion of the SLC14A1 cDNA sequence of a nucleic acid molecule in the sample, wherein the portion sequenced includes the positions corresponding to positions 226 to 228 according to SEQ ID NO:9; and/or sequencing a portion of the SLC14A1 cDNA sequence of a nucleic acid molecule in the sample, wherein the portion sequenced includes the positions corresponding to positions 394 to 396 according to SEQ ID NO:10. Any of the nucleic acid molecules disclosed herein (e.g., genomic DNA, mRNA, or cDNA) can be sequenced. In some embodiments, the detecting step comprises sequencing the entire nucleic acid molecule.

In some embodiments, the detecting step comprises: amplifying at least a portion of the nucleic acid molecule that encodes an SLC14A1 protein, wherein the amplified nucleic acid molecule encodes an amino acid sequence which comprises the position corresponding to position 76 according to SEQ ID NO:13 or comprises the position corresponding to position 132 according to SEQ ID NO:14; labeling the nucleic acid molecule with a detectable label; contacting the labeled nucleic acid with a support comprising a probe, wherein the probe comprises a nucleic acid sequence which hybridizes under stringent conditions to a nucleic acid sequence encoding an isoleucine at the position corresponding to position 76 according to SEQ ID NO:13 or encoding an isoleucine at the position corresponding to position 132 according to SEQ ID NO:14; and detecting the detectable label. Any of the nucleic acid molecules disclosed herein can be amplified. For example, any of the genomic DNA, cDNA, or mRNA molecules disclosed herein can be amplified. In some embodiments, the nucleic acid molecule is mRNA and the method further comprises reverse-transcribing the mRNA into a cDNA prior to the amplifying step.

In some embodiments, the assay comprises: a) contacting the sample with a primer hybridizing to: i) a portion of the SLC14A1 genomic sequence that is proximate to the positions of the SLC14A1 genomic sequence corresponding to positions 6963 to 6965 according to SEQ ID NO:2; ii) a portion of the SLC14A1 mRNA sequence that is proximate to the positions of the SLC14A1 mRNA corresponding to positions 226 to 228 according to SEQ ID NO:5 or corresponding to positions 394 to 396 according to SEQ ID NO:6; or iii) a portion of the SLC14A1 cDNA sequence that is proximate to the positions of the SLC14A1 cDNA corresponding to positions 226 to 228 according to SEQ ID NO:9 or corresponding to positions 394 to 396 according to SEQ ID NO:10; b) extending the primer at least through: i) the positions of the SLC14A1 genomic nucleic acid sequence corresponding to positions 6963 to 6965 according to SEQ ID NO:2; ii) the position of the SLC14A1 mRNA nucleic acid sequence corresponding to positions 226 to 228 according to SEQ ID NO:5 or corresponding to positions 394 to 396 according to SEQ ID NO:6; or iii) the position of the SLC14A1 cDNA nucleic acid sequence corresponding to positions 226 to 228 according to SEQ ID NO:9 or corresponding to positions 394 to 396 according to SEQ ID NO:10; and c) determining the whether the extension product of the primer comprises nucleotides at the positions: i) corresponding to positions 6963 to 6965 of the SLC14A1 genomic nucleic acid sequence according to SEQ ID NO:2; ii) corresponding to positions 226 to 228 of the SLC14A1 mRNA nucleic acid sequence according to SEQ ID NO:5 or corresponding to positions 394 to 396 of the SLC14A1 mRNA nucleic acid sequence according to SEQ ID NO:6; or iii) corresponding to positions 226 to 228 of the SLC14A1 cDNA nucleic acid sequence according to SEQ ID NO:9 or corresponding to positions 394 to 396 of the SLC14A1 cDNA nucleic acid sequence according to SEQ ID NO:10; that encode an isoleucine at the position corresponding to position 76 according to SEQ ID NO:13 or that encode an isoleucine at the position corresponding to position 132 according to SEQ ID NO:14.

In some embodiments, the assay comprises contacting the sample with a primer or probe that specifically hybridizes to the SLC14A1 variant genomic nucleic acid sequence, the SLC14A1 variant mRNA nucleic acid sequence, or the SLC14A1 variant cDNA nucleic acid sequence and not to the corresponding wild-type SLC14A1 nucleic acid sequence under stringent conditions, and determining whether hybridization has occurred. In some embodiments, the SLC14A1 variant genomic nucleic acid sequence, SLC14A1 variant mRNA nucleic acid sequence, or SLC14A1 variant cDNA nucleic acid encodes an amino acid sequence comprising an isoleucine at the position corresponding to position 76 according to SEQ ID NO:13 or encodes an amino acid sequence comprising an isoleucine at the position corresponding to position 132 according to SEQ ID NO:14. In some embodiments, the method is an in vitro method.

The disclosure also provides methods of determining a human subject's susceptibility to developing a coagulation condition or coronary artery disease (CAD), comprising: a) assaying a sample obtained from the human subject to determine whether an SLC14A1 protein in the sample comprises an isoleucine at the position corresponding to position 76 according to SEQ ID NO:13 and/or whether an SLC14A1 protein in the sample comprises an isoleucine at the position corresponding to position 132 according to SEQ ID NO:14; and b) classifying the human subject as being at decreased risk for developing the coagulation condition or CAD if an SLC14A1 protein in the sample comprises an isoleucine at the position corresponding to position 76 according to SEQ ID NO:13 and/or if an SLC14A1 protein in the sample comprises an isoleucine at the position corresponding to position 132 according to SEQ ID NO:14, or classifying the human subject as being at increased risk for developing the coagulation condition or CAD if an SLC14A1 protein in the sample does not comprise an isoleucine at the position corresponding to position 76 according to SEQ ID NO:13 and/or if an SLC14A1 protein in the sample does not comprise an isoleucine at the position corresponding to position 132 according to SEQ ID NO:14. In some embodiments, an enzyme-linked immunosorbent assay (ELISA) is used for determining whether an SLC14A1 protein in the sample comprises an isoleucine at the position corresponding to position 76 according to SEQ ID NO:13 and/or whether an SLC14A1 protein in the sample comprises an isoleucine at the position corresponding to position 132 according to SEQ ID NO:14. In some embodiments, the method is an in vitro method.

In some embodiments of the method, the detecting step comprises sequencing at least a portion of the nucleic acid molecule that encodes an SLC14A1 protein. The sequenced nucleic acid molecule may encode a loss of function SLC14A1 protein or a partial loss of function SLC14A1 protein. In some embodiments, the sequenced nucleic acid molecule may encode an amino acid sequence which comprises a position corresponding to position 76 according to SEQ ID NO:13 or comprises a position corresponding to position 132 according to SEQ ID NO:14. The presence of an adenine at a position corresponding to position 6963 according to SEQ ID NO:2 (e.g., the genomic DNA), or at a position corresponding to position 226 according to SEQ ID NO:5 or SEQ ID NO:9 (e.g., the mRNA), or at a position corresponding to position 394 according to SEQ ID NO:6 or SEQ ID NO:10 (e.g., the cDNA), each results in a variant SLC14A1 protein comprising an isoleucine at a position corresponding to position 76 according to SEQ ID NO:13 or a variant SLC14A1 protein comprising an isoleucine at a position corresponding to position 132 according to SEQ ID NO:14. The detecting step may comprise sequencing the nucleic acid molecule encoding the entire SLC14A1 protein.

In some embodiments of the method, the detecting step comprises amplifying at least a portion of the nucleic acid molecule that encodes an SLC14A1 protein, labeling the nucleic acid molecule with a detectable label, contacting the labeled nucleic acid with a support comprising a probe, wherein the probe comprises a nucleic acid sequence which specifically hybridizes, including, for example, under stringent conditions, to a nucleic acid sequence encoding an isoleucine at the position corresponding to position 76 according to SEQ ID NO:13 or to a nucleic acid sequence encoding an isoleucine at the position corresponding to position 132 according to SEQ ID NO:14 (or a nucleic acid sequence having an adenine at a position corresponding to position 6963 according to SEQ ID NO:2 (e.g., the genomic DNA), or at a position corresponding to position 226 according to SEQ ID NO:5 or SEQ ID NO:9 (e.g., the mRNA), or at a position corresponding to position 394 according to SEQ ID NO:6 or SEQ ID NO:10 (e.g., the cDNA), and detecting the detectable label. The amplified nucleic acid molecule preferably encodes an amino acid sequence which comprises the position corresponding to position 76 according to SEQ ID NO:13 or preferably encodes an amino acid sequence which comprises the position corresponding to position 132 according to SEQ ID NO:14. If the nucleic acid includes mRNA, the method may further comprise reverse-transcribing the mRNA into a cDNA prior to the amplifying step. In some embodiments, the determining step comprises contacting the nucleic acid molecule with a probe comprising a detectable label and detecting the detectable label. The probe preferably comprises a nucleic acid sequence which specifically hybridizes, including, for example, under stringent conditions, to a nucleic acid sequence encoding an amino acid sequence which comprises an isoleucine at the position corresponding to position 76 according to SEQ ID NO:13 or to a nucleic acid sequence encoding an amino acid sequence which comprises an isoleucine at the position corresponding to position 132 according to SEQ ID NO:14 (or a nucleic acid sequence having an adenine at a position corresponding to position 6963 according to SEQ ID NO:2 (e.g., the genomic DNA), or at a position corresponding to position 226 according to SEQ ID NO:5 or SEQ ID NO:9 (e.g., the mRNA), or at a position corresponding to position 394 according to SEQ ID NO:6 or SEQ ID NO:10 (e.g., the cDNA). The nucleic acid molecule may be present within a cell obtained from the human subject.

Other assays that can be used in the methods disclosed herein include, for example, reverse transcription polymerase chain reaction (RT-PCR) or quantitative RT-PCR (qRT-PCR). Yet other assays that can be used in the methods disclosed herein include, for example, RNA sequencing (RNA-Seq) followed by detection of the presence and quantity of variant mRNA or cDNA in the biological sample.

The methods described herein may be carried out in vitro, in situ, or in vivo.

The disclosure also provides methods of determining a human subject's susceptibility to developing a coagulation condition or CAD comprising: a) performing an assay on a sample obtained from the human subject to determine whether an SLC14A1 protein in the sample is a loss of function protein or partial loss of function protein; and b) classifying the human subject as being at decreased risk for developing the coagulation condition or CAD if the SLC14A1 polypeptide is a loss of function protein or partial loss of function protein, or classifying the human subject as being at increased risk for developing the coagulation condition or CAD if the SLC14A1 polypeptide is not a loss of function protein or partial loss of function protein.

The disclosure also provides methods of determining a human subject's susceptibility to developing a coagulation condition or CAD comprising: a) performing an assay on a sample obtained from the human subject to determine whether an SLC14A1 protein in the sample comprises an isoleucine at a position corresponding to position 76 according to SEQ ID NO:13 or comprises an isoleucine at a position corresponding to position 132 according to SEQ ID NO:14; and b) classifying the human subject as being at decreased risk for developing the coagulation condition or CAD if the SLC14A1 polypeptide comprises an isoleucine at the position corresponding to position 76 according to SEQ ID NO:13 or comprises an isoleucine at a position corresponding to position 132 according to SEQ ID NO:14, or classifying the human subject as being at increased risk for developing the coagulation condition or CAD if the SLC14A1 polypeptide does not comprise an isoleucine at the position corresponding to position 76 according to SEQ ID NO:13 or comprises an isoleucine at a position corresponding to position 132 according to SEQ ID NO:14. In some embodiments, the human subject is in need of such determination. In some embodiments, the human subject may have relatives that have a coagulation condition or CAD.

The disclosure also provides methods of determining a human subject's susceptibility to developing a coagulation condition or coronary artery disease (CAD), comprising: a) assaying a sample obtained from the human subject to determine whether a nucleic acid molecule in the sample comprises a nucleic acid sequence that encodes an SLC14A1 protein comprising an isoleucine at the position corresponding to position 76 according to SEQ ID NO:13 and/or whether a nucleic acid molecule in the sample comprises a nucleic acid sequence that encodes an SLC14A1 protein comprising an isoleucine at the position corresponding to position 132 according to SEQ ID NO:14; and b) classifying the human subject as being at decreased risk for developing the coagulation condition or CAD if a nucleic acid molecule in the sample comprises a nucleic acid sequence that encodes an SLC14A1 protein comprising an isoleucine at the position corresponding to position 76 according to SEQ ID NO:13 and/or if a nucleic acid molecule in the sample comprises a nucleic acid sequence that encodes an SLC14A1 protein comprising an isoleucine at the position corresponding to position 132 according to SEQ ID NO:14, or classifying the human subject as being at increased risk for developing the coagulation condition or CAD if a nucleic acid molecule in the sample encodes an SLC14A1 protein which does not comprise an isoleucine at the position corresponding to position 76 according to SEQ ID NO:13 and/or if a nucleic acid molecule in the sample encodes an SLC14A1 protein which does not comprise an isoleucine at the position corresponding to position 132 according to SEQ ID NO:14.

Any of the methods described herein may further comprise, for a subject having a coagulation condition or an increased risk for developing a coagulation condition, administering a therapeutic agent that prevents, treats, or inhibits (partially or completely) the coagulation condition. In some embodiments, the anti-coagulation agent is heparin, warfarin (COUMADIN® and JANTOVEN®), rivaroxaban (XARELTO®), dabigatran (PRADAXA®), apixaban (ELIQUIS®), edoxaban (SAVAYSA®), enoxaparin (LOVENOX®), fondaparinux (ARIXTRA®), dalteparin (FRAGMIN®), bivalirudin (ANGIOMAX®), argatroban (ACOVA®), or antithrombin III (THROMBATE III®). In some embodiments, the anti-coagulation agent is any of the variant SLC14A1 polypeptides described herein.

Any of the methods described herein may further comprise, for a subject having CAD or an increased risk for developing CAD, administering a therapeutic agent that prevents, treats, or inhibits (partially or completely) CAD. In some embodiments, the agent is a cholesterol-modifying medication (such as, for example, a statin, niacin, a fibrate, or a bile acid sequestrant), aspirin, a beta blocker, nitroglycerin, an angiotensin-converting enzyme (ACE) inhibitor, and/or an angiotensin II receptor blocker (ARB).

The disclosure also provides methods for treating a coagulation condition patient with a therapeutic agent that prevents, treats, or inhibits the coagulation condition, comprising the steps of: determining whether the patient has one or more genetic variants associated with the coagulation condition by performing or having performed a genotype assay on a DNA sample obtained from the patient to determine if the patient has one or more genetic variants associated with the coagulation condition; and when the patient has one or more of the genetic variants associated with the coagulation condition, administering to the patient the therapeutic agent that prevents, treats, or inhibits the coagulation condition. The genetic variants associated with the coagulation condition can be any of the variants disclosed herein with such activity. In some embodiments, the one or more genetic variants associated with the coagulation condition is a nucleic acid molecule that encodes an SLC14A1 protein which does not comprise an isoleucine at the position corresponding to position 76 according to SEQ ID NO:13 and/or a nucleic acid molecule that encodes an SLC14A1 protein which does not comprise an isoleucine at the position corresponding to position 132 according to SEQ ID NO:14. The determining of whether the patient has one or more genetic variants associated with the coagulation condition by performing or having performed a genotype assay can encompass any of the methods described herein. In some embodiments, when the genotype assay indicates that the coagulation condition patient comprises a nucleic acid molecule that encodes an SLC14A1 protein which comprises an isoleucine at the position corresponding to position 76 according to SEQ ID NO:13 and/or a nucleic acid molecule that encodes an SLC14A1 protein which comprises an isoleucine at the position corresponding to position 132 according to SEQ ID NO:14, the coagulation condition patient is treated with a therapeutic agent that prevents, treats, or inhibits the coagulation condition, but at a dose that is lower or less frequent (e.g., about 10% lower or less frequent, about 20% lower or less frequent, about 30% lower or less frequent, about 40% lower or less frequent, about 50% lower or less frequent, about 60% lower or less frequent, or about 70% lower or less frequent), than if the coagulation condition patient comprises a nucleic acid molecule that encodes an SLC14A1 protein which does not comprise an isoleucine at the position corresponding to position 76 according to SEQ ID NO:13 and/or a nucleic acid molecule that encodes an SLC14A1 protein which does not comprise an isoleucine at the position corresponding to position 132 according to SEQ ID NO:14. In some embodiments, the therapeutic agent that prevents, treats, or inhibits the coagulation condition is heparin, warfarin (COUMADIN® and JANTOVEN®), rivaroxaban (XARELTO®), dabigatran (PRADAXA®), apixaban (ELIQUIS®), edoxaban (SAVAYSA®), enoxaparin (LOVENOX®), fondaparinux (ARIXTRA®), dalteparin (FRAGMIN®), bivalirudin (ANGIOMAX®), argatroban (ACOVA®), or antithrombin III (THROMBATE III®).

The disclosure also provides methods for treating a coagulation condition patient with a therapeutic agent that prevents, treats, or inhibits the coagulation condition, comprising the steps of: determining whether the patient has one or more genetic variants associated with the coagulation condition by performing or having performed an assay on a protein sample obtained from the patient to determine if the patient has one or more genetic variants associated with the coagulation condition; and when the patient has one or more of the genetic variants associated with the coagulation condition, administering to the patient the therapeutic agent that prevents, treats, or inhibits the coagulation condition. The genetic variants associated with the coagulation condition can be any of the variants disclosed herein with such activity. In some embodiments, the one or more genetic variants associated with the coagulation condition is an SLC14A1 protein which does not comprise an isoleucine at the position corresponding to position 76 according to SEQ ID NO:13 and/or an SLC14A1 protein which does not comprise an isoleucine at the position corresponding to position 132 according to SEQ ID NO:14. The determining of whether the patient has one or more genetic variants associated with the coagulation condition by performing or having performed an assay can encompass any of the methods described herein. In some embodiments, when the assay indicates that the coagulation condition patient comprises an SLC14A1 protein which comprises an isoleucine at the position corresponding to position 76 according to SEQ ID NO:13 and/or an SLC14A1 protein which comprises an isoleucine at the position corresponding to position 132 according to SEQ ID NO:14, the coagulation condition patient is treated with a therapeutic agent that prevents, treats, or inhibits the coagulation condition, but at a dose that is lower or less frequent (e.g., about 10% lower or less frequent, about 20% lower or less frequent, about 30% lower or less frequent, about 40% lower or less frequent, about 50% lower or less frequent, about 60% lower or less frequent, or about 70% lower or less frequent), than if the coagulation condition patient comprises an SLC14A1 protein which does not comprise an isoleucine at the position corresponding to position 76 according to SEQ ID NO:13 and/or an SLC14A1 protein which does not comprise an isoleucine at the position corresponding to position 132 according to SEQ ID NO:14. In some embodiments, the therapeutic agent that prevents, treats, or inhibits the coagulation condition is heparin, warfarin (COUMADIN® and JANTOVEN®), rivaroxaban (XARELTO®), dabigatran (PRADAXA®), apixaban (ELIQUIS®), edoxaban (SAVAYSA®), enoxaparin (LOVENOX®), fondaparinux (ARIXTRA®), dalteparin (FRAGMIN®), bivalirudin (ANGIOMAX®), argatroban (ACOVA®), or antithrombin III (THROMBATE III®).

The disclosure also provides methods for treating a coronary artery disease (CAD) patient with a therapeutic agent that prevents, treats, or inhibits the coronary artery disease, comprising the steps of: determining whether the patient has one or more genetic variants associated with the coronary artery disease by performing or having performed a genotype assay on a DNA sample obtained from the patient to determine if the patient has one or more genetic variants associated with the coronary artery disease; and when the patient has one or more of the genetic variants associated with the coronary artery disease, administering to the patient the therapeutic agent that prevents, treats, or inhibits the coronary artery disease. The genetic variants associated with the coronary artery disease can be any of the variants disclosed herein with such activity. In some embodiments, the one or more genetic variants associated with the coronary artery disease is a nucleic acid molecule that encodes an SLC14A1 protein which does not comprise an isoleucine at the position corresponding to position 76 according to SEQ ID NO:13 and/or a nucleic acid molecule that encodes an SLC14A1 protein which does not comprise an isoleucine at the position corresponding to position 132 according to SEQ ID NO:14. The determining of whether the patient has one or more genetic variants associated with the coronary artery disease by performing or having performed a genotype assay can encompass any of the methods described herein. In some embodiments, when the genotype assay indicates that the coronary artery disease patient comprises a nucleic acid molecule that encodes an SLC14A1 protein which comprises an isoleucine at the position corresponding to position 76 according to SEQ ID NO:13 and/or a nucleic acid molecule that encodes an SLC14A1 protein which comprises an isoleucine at the position corresponding to position 132 according to SEQ ID NO:14, the coronary artery disease patient is treated with a therapeutic agent that prevents, treats, or inhibits the coronary artery disease, but at a dose that is lower or less frequent (e.g., about 10% lower or less frequent, about 20% lower or less frequent, about 30% lower or less frequent, about 40% lower or less frequent, about 50% lower or less frequent, about 60% lower or less frequent, or about 70% lower or less frequent), than if the coronary artery disease patient comprises a nucleic acid molecule that encodes an SLC14A1 protein which does not comprise an isoleucine at the position corresponding to position 76 according to SEQ ID NO:13 and/or a nucleic acid molecule that encodes an SLC14A1 protein which does not comprise an isoleucine at the position corresponding to position 132 according to SEQ ID NO:14. In some embodiments, the therapeutic agent that prevents, treats, or inhibits the coronary artery disease is a cholesterol-modifying medication, aspirin, a beta blocker, nitroglycerin, an angiotensin-converting enzyme (ACE) inhibitor, and/or an angiotensin II receptor blocker (ARB). In some embodiments, the cholesterol-modifying medication is a statin, niacin, a fibrate, or a bile acid sequestrant.

The disclosure also provides methods for treating a coronary artery disease (CAD) patient with a therapeutic agent that prevents, treats, or inhibits the coronary artery disease, comprising the steps of: determining whether the patient has one or more genetic variants associated with the coronary artery disease by performing or having performed an assay on a protein sample obtained from the patient to determine if the patient has one or more genetic variants associated with the coronary artery disease; and when the patient has one or more of the genetic variants associated with the coronary artery disease, administering to the patient the therapeutic agent that prevents, treats, or inhibits the coronary artery disease. The genetic variants associated with the coronary artery disease can be any of the variants disclosed herein with such activity. In some embodiments, the one or more genetic variants associated with the coronary artery disease is an SLC14A1 protein which does not comprise an isoleucine at the position corresponding to position 76 according to SEQ ID NO:13 and/or an SLC14A1 protein which does not comprise an isoleucine at the position corresponding to position 132 according to SEQ ID NO:14. The determining of whether the patient has one or more genetic variants associated with the coronary artery disease by performing or having performed an assay can encompass any of the methods described herein. In some embodiments, when the assay indicates that the coronary artery disease patient comprises an SLC14A1 protein which comprises an isoleucine at the position corresponding to position 76 according to SEQ ID NO:13 and/or an SLC14A1 protein which comprises an isoleucine at the position corresponding to position 132 according to SEQ ID NO:14, the coronary artery disease patient is treated with a therapeutic agent that prevents, treats, or inhibits the coronary artery disease, but at a dose that is lower or less frequent (e.g., about 10% lower or less frequent, about 20% lower or less frequent, about 30% lower or less frequent, about 40% lower or less frequent, about 50% lower or less frequent, about 60% lower or less frequent, or about 70% lower or less frequent), than if the coronary artery disease patient comprises an SLC14A1 protein which does not comprise an isoleucine at the position corresponding to position 76 according to SEQ ID NO:13 and/or an SLC14A1 protein which does not comprise an isoleucine at the position corresponding to position 132 according to SEQ ID NO:14. In some embodiments, the therapeutic agent that prevents, treats, or inhibits the coronary artery disease is a cholesterol-modifying medication, aspirin, a beta blocker, nitroglycerin, an angiotensin-converting enzyme (ACE) inhibitor, and/or an angiotensin II receptor blocker (ARB). In some embodiments, the cholesterol-modifying medication is a statin, niacin, a fibrate, or a bile acid sequestrant.

Administration of the treatment agents can be by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

In any of the embodiments described herein, the methods can be used for the detection, diagnosis, identification, and/or treatment of a subject having or at risk of having a coagulation condition and/or CAD. In any of the embodiments described herein, the methods can be used for the detection, diagnosis, identification, and/or treatment of a subject having or at risk of having a coagulation condition. In any of the embodiments described herein, the methods can be used for the detection, diagnosis, identification, and/or treatment of a subject having or at risk of having CAD. In some embodiments, the coagulation condition is chosen from thrombosis, pulmonary embolism, myocardial infarction (MI), venous thromboembolism (VTE), deep vein thrombosis (DVT), cerebral aneurysm, and stroke. In some embodiments, the methods are not used for the detection, diagnosis, identification, and/or treatment of a subject having or at risk of having or needing a hematopoiesis condition.

The disclosure also provides an anti-coagulation agent for use in the treatment of a coagulation condition in a human subject having a variant SLC14A1 protein, wherein the variant SLC14A1 protein is a loss of function SLC14A1 protein or a partial loss of function SLC14A1 protein. In some embodiments, the anti-coagulation agent is for use in the treatment of a coagulation condition in a human subject having a variant SLC14A1 protein that does not comprise an isoleucine at a position corresponding to position 76 according to SEQ ID NO:13 or that does not comprise an isoleucine at a position corresponding to position 132 according to SEQ ID NO:14. In some embodiments, the human subject has been tested positive for an SLC14A1 protein that does not comprise an isoleucine at a position corresponding to position 76 according to SEQ ID NO:13 or that does not comprise an isoleucine at a position corresponding to position 132 according to SEQ ID NO:14 and/or for a nucleic acid molecule encoding the SLC14A1 protein. In some embodiments, the treatment comprises the step of determining whether or not the human subject has an SLC14A1 protein that does not comprise an isoleucine at a position corresponding to position 76 according to SEQ ID NO:13 or that does not comprise an isoleucine at a position corresponding to position 132 according to SEQ ID NO:14 and/or a nucleic acid molecule encoding the SLC14A1 protein. In some embodiments, the human subject has been identified as having a coagulation condition or as having a risk for developing a coagulation condition by using any of the methods described herein. In some embodiments, the anti-coagulation agent is heparin, warfarin (COUMADIN® and JANTOVEN®), rivaroxaban (XARELTO®), dabigatran (PRADAXA®), apixaban (ELIQUIS®), edoxaban (SAVAYSA®), enoxaparin (LOVENOX®), fondaparinux (ARIXTRA®), dalteparin (FRAGMIN®), bivalirudin (ANGIOMAX®), argatroban (ACOVA®), or antithrombin III (THROMBATE III®). In some embodiments, the anti-coagulation agent is any of the variant SLC14A1 polypeptides described herein.

The disclosure also provides uses of any of the variant SLC14A1 genomic DNA, mRNA, cDNA, polypeptides, and hybridizing nucleic acid molecules disclosed herein for determining a subject's susceptibility to develop a coagulation condition.

The disclosure also provides an agent for use in the treatment of CAD in a human subject having a variant SLC14A1 protein, wherein the variant SLC14A1 protein is a loss of function SLC14A1 protein or a partial loss of function SLC14A1 protein. In some embodiments, the anti-CAD agent is for use in the treatment of CAD in a human subject having a variant SLC14A1 protein that does not comprise an isoleucine at a position corresponding to position 76 according to SEQ ID NO:13 or that does not comprise an isoleucine at a position corresponding to position 132 according to SEQ ID NO:14. In some embodiments, the human subject has been tested positive for an SLC14A1 protein that does not comprise an isoleucine at a position corresponding to position 76 according to SEQ ID NO:13 or that does not comprise an isoleucine at a position corresponding to position 132 according to SEQ ID NO:14 and/or for a nucleic acid molecule encoding the SLC14A1 protein. In some embodiments, the treatment comprises the step of determining whether or not the human subject has an SLC14A1 protein that does not comprise an isoleucine at a position corresponding to position 76 according to SEQ ID NO:13 or that does not comprise an isoleucine at a position corresponding to position 132 according to SEQ ID NO:14 and/or a nucleic acid molecule encoding the SLC14A1 protein. In some embodiments, the human subject has been identified as having CAD or as having a risk for developing CAD by using any of the methods described herein. In some embodiments, the agent is a cholesterol-modifying medication (such as, for example, a statin, niacin, a fibrate, or a bile acid sequestrant), aspirin, a beta blocker, nitroglycerin, an angiotensin-converting enzyme (ACE) inhibitor, and/or an angiotensin II receptor blocker (ARB). In some embodiments, the agent is any of the variant SLC14A1 polypeptides described herein.

The disclosure also provides uses of any of the variant SLC14A1 genomic DNA, mRNA, cDNA, polypeptides, and hybridizing nucleic acid molecules disclosed herein for determining a subject's susceptibility to develop a coagulation condition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other feature, step, element, embodiment, or aspect unless specifically indicated otherwise. Although the disclosure has been described in some detail by way of illustration and example for purposes of clarity and understanding, it will be apparent that certain changes and modifications may be practiced within the scope of the appended claims.

The nucleotide and amino acid sequences recited herein are shown using standard letter abbreviations for nucleotide bases, and one-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

The following examples are provided to describe the embodiments in greater detail. They are intended to illustrate, not to limit, the claimed embodiments.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Patient Recruitment and Phenotyping

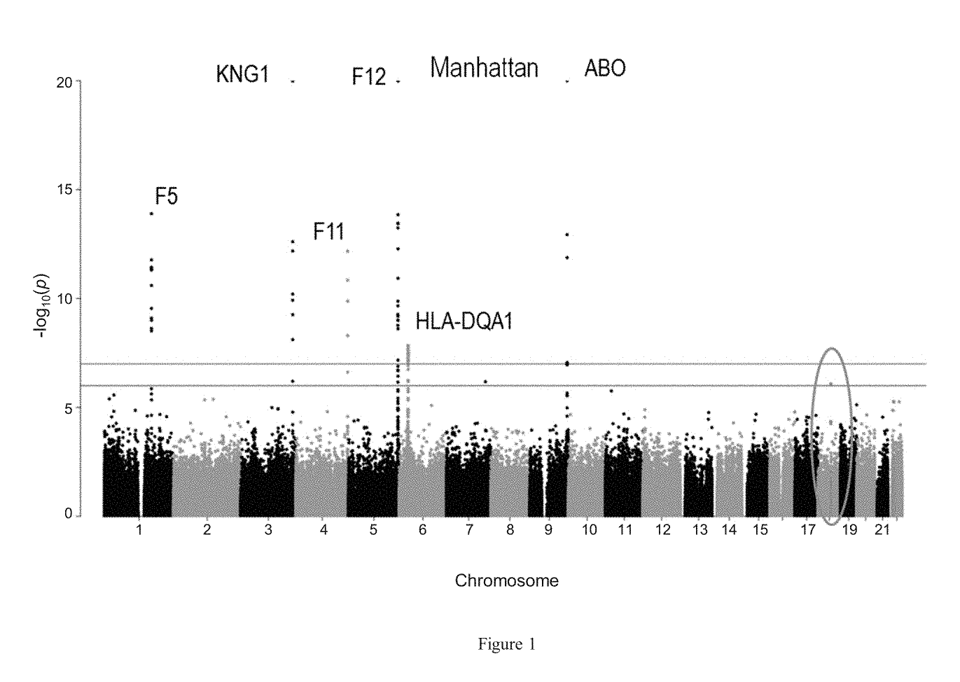

The MyCode Community Health Initiative is a cohort of more than 125,000 Geisinger Health System (GHS) patients who have consented to provide access to de-identified electronic health records (EHR) and genomic information for research purposes. As part of the DiscovEHR collaboration between Regeneron Genetics Center and GHS, whole exome sequencing was completed in more than 90,000 GHS participants of largely European-descent. In the first phase of this coagulation study, a genetic association study for activated partial thromboplastin time, an ex vivo measure of the intrinsic coagulation pathway, was completed in 17,630 European-descent individuals (see, FIG. 1). Since many patients had multiple aPTT measurements recorded, the minimum lifetime measure of aPTT for each patient was selected (to minimize the potential influence of anticoagulant usage), and all individuals with a history of venous thromboembolism were excluded from analysis. To replicate findings from this discovery analysis, aPTT was analyzed in an additional 5,892 European-descent GHS participants. Since hypercoagulability is a potential risk factor for venous and arterial thrombosis, we also evaluated the contribution of SLC14A1 V76I to coronary artery disease (CAD) risk in 96,180 individuals (African American and European-descent individuals drawn from GHS and two additional studies sequenced at the Regeneron Genetics Center, as well as the contribution of an SLC14A1 predicted loss-of-function variant (c.510-1G>A) to CAD risk in 13,963 Taiwanese individuals also sequenced at the Regeneron Genetics Center.

### Example 2: Genomic Samples

Genomic DNA was extracted from peripheral blood samples and transferred to the Regeneron Genetics Center (RGC) for whole exome sequencing, and stored in automated biobanks at −80° C. Fluorescence-based quantification was performed to ensure appropriate DNA quantity and quality for sequencing purposes.

1 μg of DNA was sheared to an average fragment length of 150 base pairs (Covaris LE220) and prepared for exome capture with a custom reagent kit from Kapa Biosystems. Samples were captured using the NimbleGen SeqCap VCRome 2.1 or the Integrated DNA Technologies xGen exome target designs. Samples were barcoded, pooled, and multiplexed for sequenced using 75 bp paired-end sequencing on an Illumina HiSeq 2500 with v4 chemistry. Captured fragments were sequenced to achieve a minimum of 85% of the target bases covered at 20× or greater coverage. Following sequencing, data was processed using a cloud-based pipeline developed at the RGC that uses DNAnexus and AWS to run standard tools for sample-level data production and analysis. Briefly, sequence data were generated and de-multiplexed using Illumina's CASAVA software. Sequence reads were mapped and aligned to the GRCh38 human genome reference assembly using BWA-mem. After alignment, duplicate reads were marked and flagged using Picard tools and indels were realigned using GATK to improve variant call quality. SNP and INDEL variants and genotypes were called using GATK's HaplotypeCaller and Variant Quality Score Recalibration (VQSR) from GATK was applied to annotate the overall variant quality scores. Sequencing and data quality metric statistics were captured for each sample to evaluate capture performance, alignment performance, and variant calling.

### Example 3: Genomic Data Analyses

Standard quality-control filters for minimum read depth (>10), genotype quality (>30), and allelic balance (>15%) were applied to called variants. Passing variants were classified and annotated based on their potential functional effects (whether synonymous, nonsynonymous, splicing, frameshift, or non-frameshift variants) using an RGC developed annotation and analysis pipeline. Familial relationships were verified through identity by descent (IBD) derived metrics from genetic data to infer relatedness and relationships in the cohort using PRIMUS (Staples et al., Amer. J. Human Genet., 2014, 95, 553-564) and cross-referencing with the reported pedigree for this family.

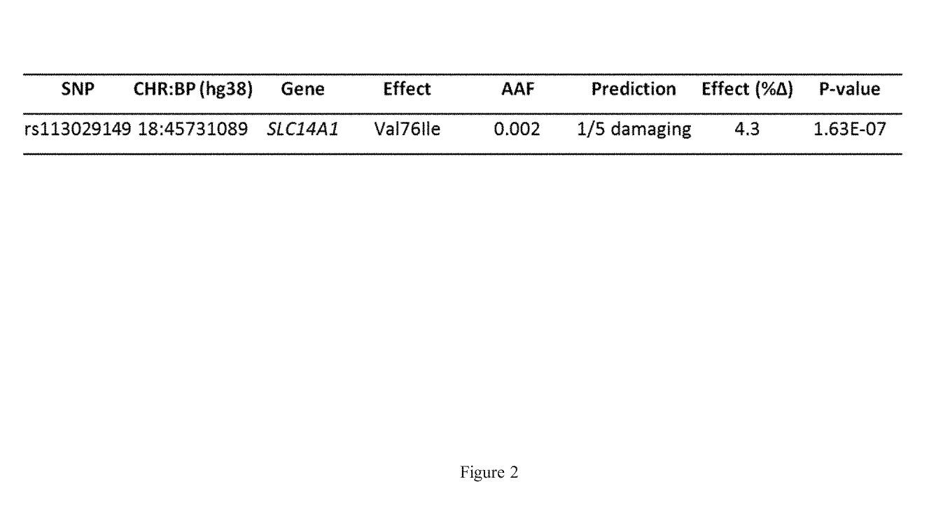

An exome-wide association analysis (exWAS) was conducted for aPTT in our discovery cohort assuming an additive model of inheritance (0, 1, or 2 copies of risk allele). We used Mixed Models Analysis in Pedigrees (MMAP) to perform linear mixed models for all variants with a minor allele count >=8, with covariate adjustment for age, age-squared, sex, and first four principal components to account for population stratification. For the first-round of analysis, signals were selected for follow-up if they had a P≤1×10⁻⁶. In addition to replicating several well-established association signals for aPTT, a novel association (P=8.4×10⁻⁷) was identified with an SLC14A1 missense variant (V76I) that is rare in Europeans (MAF=0.002), but found more commonly in African Americans (MAF=0.07) (FIGS. 1 and 2).

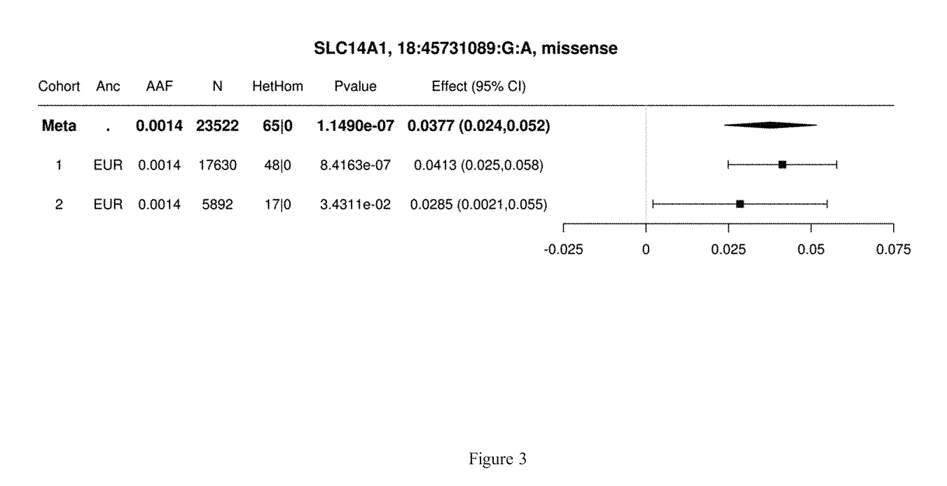

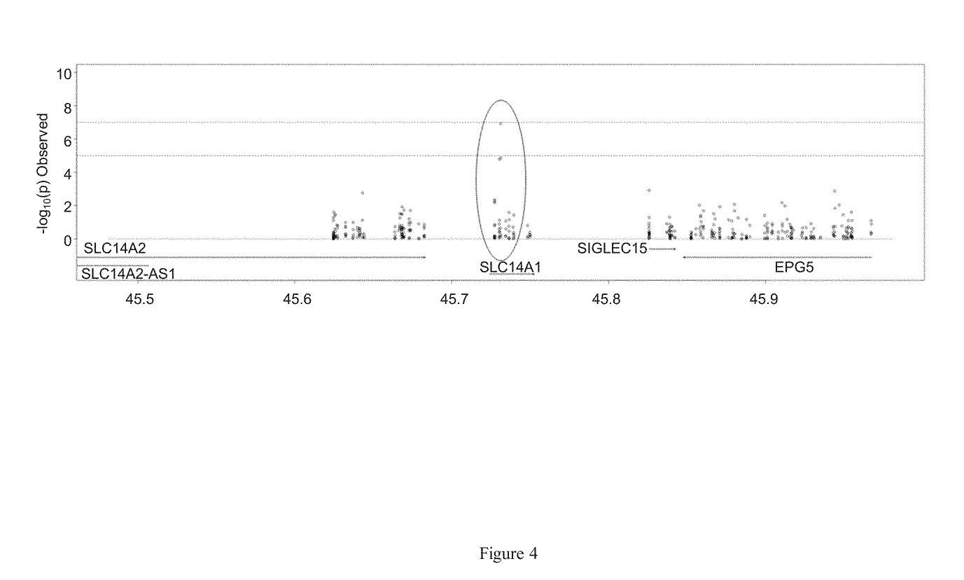

To provide additional support for this finding, we performed analysis in an independent subset of 5,892 European-descent GHS participants and conducted a meta-analysis of association statistics for the discovery and replication cohorts using fixed-effects inverse variance weighting using PLINK v1.9. We observed a nominally significant association in the replication cohort (P=0.035) and strong evidence for association with increased clotting time in the overall meta-analysis (P=1.1×10⁻⁷) (FIGS. 3 and 4).

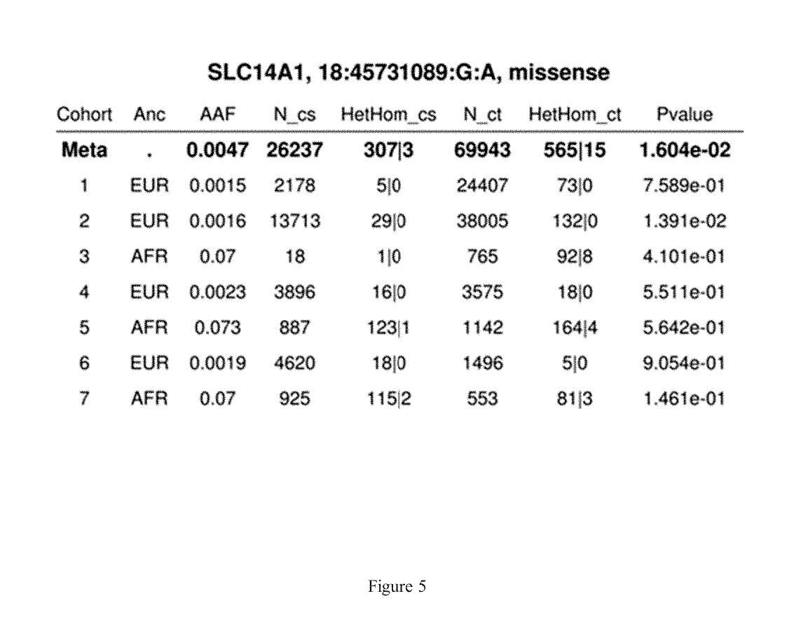

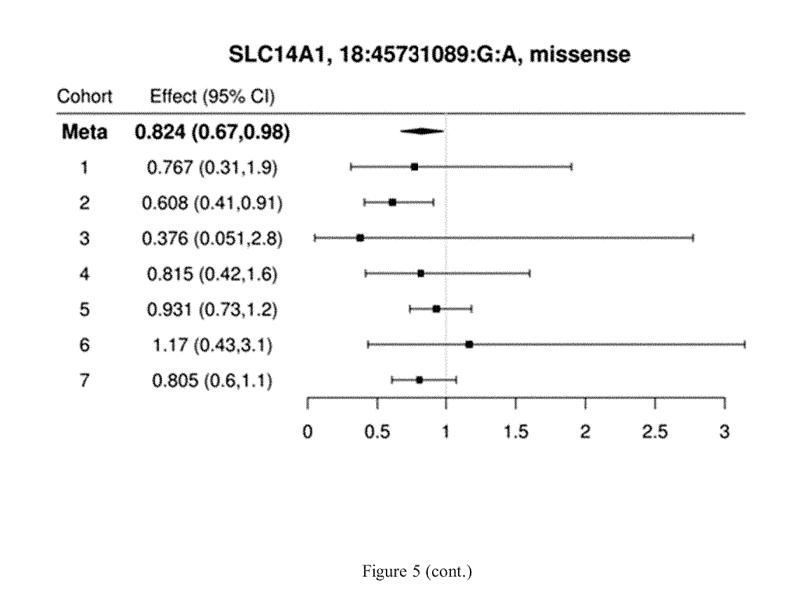

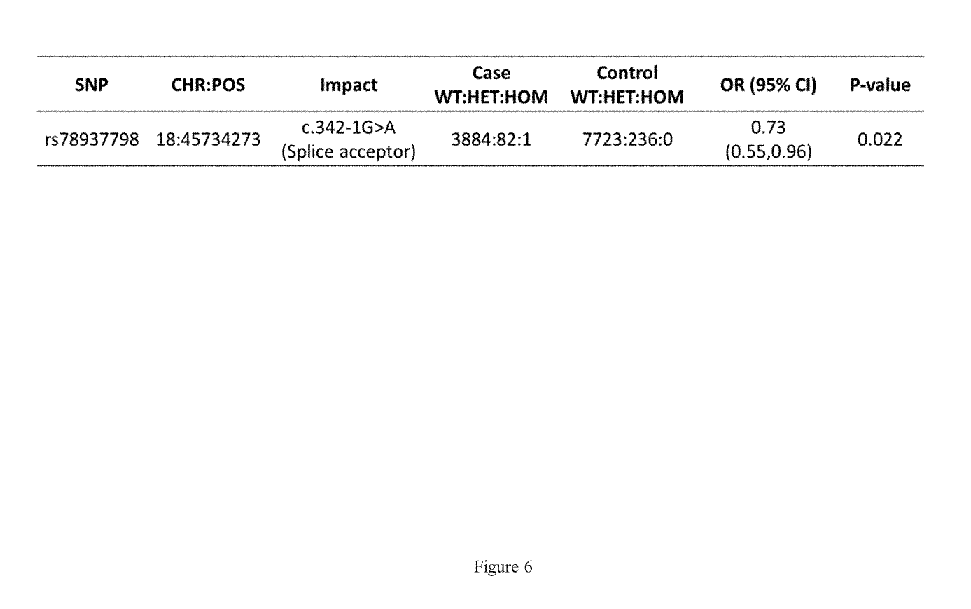

To evaluate the clinical relevance of SLC14A1 V76I, we conducted a Fisher's Exact Test for association with measures of thrombosis (CAD) in 96,180 multi-ethnic individuals with genotype and phenotype data. SLC14A1 V76I association with CAD was evaluated independently in seven different datasets (1: 2,178/24,407 European-ancestry CAD cases/controls from the GHS dataset; 2: 13,713/38,005 additional European-ancestry CAD cases/controls from the GHS dataset; 3: 18/765 African-American CAD cases/controls from the GHS dataset; 4: 3,896/3,575 independent European-ancestry cases/controls; 5: 887/1,142 independent African-American cases/controls; 6: 4,620/1,496 independent European-ancestry cases/controls; 7: 925/553 independent African-American cases/controls) and summary statistics were meta-analyzed using a fixed-effects inverse variance weighting with PLINK v1.9. Overall, SLC14A1 V76I demonstrated a protective effect for CAD across these seven cohorts (P=0.016, B=0.81) (FIG. 5). Additionally, we used logistic regression to evaluate an association between CAD and an SLC14A1 predicted loss-of-function variant in a Taiwanese cohort (c.510-1G>A, 374 heterozygotes, 1 minor allele homozygote). We noted SLC14A1 c.510-1G>A carriers to have reduced risk of CAD as compared to non-carriers (P=0.02, OR=0.71) (FIG. 6).

### Example 4: Detection

The presence of a certain genetic variant in a subject can indicate that the subject has an increased risk of having or developing a coagulopathy or coronary artery disease. A sample, such as a blood sample, can be obtained from a subject. Nucleic acids can be isolated from the sample using common nucleic acid extraction kits. After isolating the nucleic acid from the sample obtained from the subject, the nucleic acid is sequenced to determine if there is a genetic variant present. The sequence of the nucleic acid can be compared to a control sequence (wild type sequence). Finding a difference between the nucleic acid obtained from the sample obtained from the subject and the control sequence indicates the presence of a genetic variant. These steps can be performed as described in the examples above and throughout the disclosure. The presence of one or more genetic variants is indicative of the subject's increased risk for having or developing thrombotic events or coronary artery disease.

What is claimed:

1. A cDNA encoding a human Solute Carrier Family 14 Member 1 (SLC14A1) protein, comprising a nucleic acid sequence which is:
at least about 90%, at least about 95%, at least about 96%, at least about 97%, at least about 98%, or at least about 99% identical to SEQ ID NO:9, provided that the nucleic acid sequence encodes an amino acid sequence which comprises an isoleucine at the position corresponding to position 76 according to SEQ ID NO:13, or the complement thereof; or
at least about 90%, at least about 95%, at least about 96%, at least about 97%, at least about 98%, or at least about 99% identical to SEQ ID NO:10, provided that the nucleic acid sequence encodes an amino acid sequence which comprises isoleucine at the position corresponding to position 132 according to SEQ ID NO:14, or the complement thereof.

2. The cDNA according to claim 1, wherein the nucleic acid sequence comprises SEQ ID NO:9.

3. The cDNA according to claim 86, wherein the nucleic acid sequence comprises SEQ ID NO:10.

4. A vector comprising the cDNA according to claim 1.

5. The vector according to claim 4, wherein the vector comprises a plasmid.

6. The vector according to claim 4, wherein the vector comprises a virus.

7. A composition comprising the cDNA according to claim 1 and a carrier.

8. A composition comprising the vector according to claim 4 and a carrier.

9. A host cell comprising the cDNA according to claim 1.

10. A host cell comprising the vector according to claim 4.

11. The host cell according to claim 9, wherein the cDNA is operably linked to a promoter active in the host cell.

12. The host cell according to claim 11, wherein the promoter is an inducible promoter.

13. The host cell according to claim 9, wherein the host cell is a bacterial cell, a yeast cell, or an insect cell.

14. The host cell according to claim 9, wherein the host cell is a mammalian cell.

15. An isolated alteration-specific probe or primer comprising at least about 15 nucleotides and which hybridizes to a nucleic acid sequence encoding an SLC14A1 protein, wherein the alteration-specific probe or primer comprises:
a nucleic acid sequence which is complementary to the portion of the SLC14A1 encoding nucleic acid sequence which encodes an isoleucine at the position corresponding to position 76 according to SEQ ID NO:13, or to the complement thereof; or
a nucleic acid sequence which is complementary to the portion of the SLC14A1 encoding nucleic acid sequence which encodes an isoleucine at the position corresponding to position 132 according to SEQ ID NO:14, or to the complement thereof.

16. An isolated alteration-specific probe or primer comprising a nucleic acid sequence which is complementary to a nucleic acid sequence encoding an SLC14A1 protein having an isoleucine at the position corresponding to position 76 according to SEQ ID NO:13 and/or which is complementary to a nucleic acid sequence encoding an SLC14A1 protein having an isoleucine at the position corresponding to position 132 according to SEQ ID NO:14, wherein the alteration-specific probe or primer comprises a nucleic acid sequence which is complementary to a portion of the nucleic acid sequence comprising the positions corresponding to: positions 6963 to 6965 according to SEQ ID NO:2, or the complement thereof; positions 226 to 228 according to SEQ ID NO:5, or the complement thereof; positions 394 to 396 according to SEQ ID NO:6, or the complement thereof; positions 226 to 228 according to SEQ ID NO:9, or the complement thereof; positions 394 to 396 according to SEQ ID NO:10, or the complement thereof.
